SEQUENCE LISTING (8 sequences)

SEQ ID NO: 1 - DNA, 58 bp, Spinacia oleracea
  source 1..58: mol_type = genomic DNA
cctaatggctctaaggtttcatcaagacctaagaaagcagaaaaaatgcagaagccca

SEQ ID NO: 2 - DNA, 58 bp, Spinacia oleracea
  source 1..58: mol_type = genomic DNA
cctaatggctctaaggtttcatcaaaacctaagaaagcagaaaaaatgcagaagccca

SEQ ID NO: 3 - DNA, 80 bp, Spinacia oleracea
  source 1..80: mol_type = genomic DNA
attgtacaaatttcagaaacagttataaccaatttcagataataaacagatttccacttcacatatttcttacctcaarc

SEQ ID NO: 4 - DNA, 80 bp, Spinacia oleracea
  source 1..80: mol_type = genomic DNA
attgtacaaatttcagaaacagttataaccaaattcagataataaacagatttccacttcacatatttcttacctcaarc

SEQ ID NO: 5 - DNA, 83 bp, Spinacia oleracea
  source 1..83: mol_type = genomic DNA
cataaacattccgtatgagtagtactctatttgtctcaaaaagaaaattgaaaattgccctagtcgaaattttatctgcacta

SEQ ID NO: 6 - DNA, 82 bp, Spinacia oleracea
  source 1..82: mol_type = genomic DNA
cataaacattccgtatgagtagtactctatttgtctcaaaaagaaaattgaaaattgcctagtcgaaattttatctgcacta

SEQ ID NO: 7 - DNA, 49 bp, Spinacia oleracea
  source 1..49: mol_type = genomic DNA
gatgctcagccgctcaccagtatttggttttcatgagccaaaaactgga

SEQ ID NO: 8 - DNA, 49 bp, Spinacia oleracea
  source 1..49: mol_type = genomic DNA
gatgctcagccgctcaccagtatctggttttcatgagccaaaaactgga

(19) United States
(12) Patent Application Publication
(10) Pub. No.: US 2026/0130334 A1
(43) Pub. Date: May 14, 2026

(54) NON R-GENE MEDIATED PERONOSPORA RESISTANCE IN SPINACIA OLERACEA

(71) Applicant: RIJK ZWAAN ZAADTEELT EN ZAADHANDEL B.V., De Lier (NL)

(72) Inventors: Johannes Geert Jan Feitsma, De Lier (NL); Vincent Laurens Adrianus Kock, De Lier (NL); Jan Hugo Den Braber, De Lier (NL)

(21) Appl. No.: 19/421,269

(22) Filed: Dec. 16, 2025

Related U.S. Application Data
(63) Continuation-in-part of application No. 18/358,110, filed on Jul. 25, 2023, now abandoned.
Application No. 18/358,110 is a continuation of application No. 16/223,491, filed on Dec. 18, 2018, now Pat. No. 11,751,523.
Application No. 16/223,491 is a continuation of application No. 15/153,927, filed on May 13, 2016, now Pat. No. 10,226,016.

(51) Int. Cl. A01H 6/02; A01H 1/04; A01H 5/12

(52) U.S. Cl. CPC A01H 6/028; A01H 1/04; A01H 1/045; A01H 5/12; C12N 15/8216; C12N 15/8218; C12N 15/8247; C12N 15/8271; C12N 15/8286; C12Q 2600/13; C12Q 2600/156

(57) ABSTRACT

The present invention relates to a Spinacia oleracea plant comprising a nucleic acid molecule which when homozygously present provides at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, wherein said nucleic acid molecule comprises a gene which comprises a coding sequence according to SEQ ID NO: 1 and a gene which comprises a coding sequence according to SEQ ID NO: 2, or a gene which comprises a coding sequence having at least 95.0% sequence identity to SEQ ID NO: 7 and comprises an insertion of sequence motif GAAATA between nucleotides 1235 and 1236 of SEQ ID NO: 7 and a gene which comprises a coding sequence having at least 95.0% sequence identity to SEQ ID NO: 8 and lacks a sequence according to SEQ ID NO: 14, wherein the 95.0% sequence identity is over the part of SEQ ID NO: 8 without the lacking sequence, or genes comprising the corresponding genomic sequences or genes encoding a protein having an amino acid sequence according to SEQ ID NO: 3, and a gene encoding a protein having an amino acid sequence according to SEQ ID NO: 4, or genes encoding proteins showing 95% sequence identity thereto. The invention further relates to a plant carrying the nucleic acid molecule and to methods of selecting, producing and growing such plants.

### RELATED APPLICATIONS AND INCORPORATION BY REFERENCE

This application is a continuation-in-part of U.S. patent application Ser. No. 18/358,110, filed Jul. 25, 2023, which is a continuation of U.S. patent application Ser. No. 16/223,491, filed Dec. 18, 2018, now U.S. Pat. No. 11,751,523, which is a continuation of U.S. patent application Ser. No. 15/153,927, filed May 13, 2016, now U.S. Pat. No. 10,226,016.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy was created Dec. 16, 2025, is named ReplacementY7954_03281SL.xml and is 83,898 bytes in size.

### FIELD OF THE INVENTION

The invention relates to a nucleic acid molecule conferring resistance against downy mildew (Peronospora effusa), and Spinacia oleracea plants comprising said nucleic acid molecule. The invention further relates to seed and cells of said resistant spinach plant, and propagation material suitable for producing said plant. The invention further relates to methods and markers for producing such a plant, and to methods and genetic markers for selection of such a plant. The invention is also directed to a marker for identification of said nucleic acid molecule in a plant, and to use of said marker.

### BACKGROUND OF THE INVENTION

Spinach (Spinacia oleracea L.) is a flowering plant from the Amaranthaceae family that is grown as a vegetable. The consumable parts of spinach are the leaves and petioles from the vegetative stage. Spinach is sold loose, bunched, in pre-packed bags, canned, or frozen. There are three basic types of spinach: industry-, fresh and Asiatic spinach. Within these types three different leaf types can be recognized: namely the savoy, semi-savoy and smooth types. Savoy has dark green, crinkly and curly leaves. Flat or smooth leaf spinach has broad, smooth leaves. Semi-savoy is a variety with slightly crinkled leaves. The main market for spinach is baby-leaf Baby spinach leaves are often of the flat-leaf variety and usually the harvested leaves are not longer than about eight centimeter. These tender, sweet leaves are sold loose rather than in bunches. They are often used in salads but can also be lightly cooked.

Downy mildew (Peronospora effusa) is a major threat for spinach growers because it directly affects the harvested leaves. In spinach, downy mildew is caused by the oomycete Peronospora effusa (formerly known as Peronospora farinosa f. sp. spinaciae). Infection makes the leaves unsuitable for sale and consumption, as it manifests itself phenotypically as yellow lesions on the older leaves, and on the abaxial leaf surface a greyish fungal growth can be observed. The infection can spread very rapidly, and it can occur both in glasshouse cultivation and in soil cultivation. The optimal temperature for formation and germination of P. effusa spores is 9 to 12° C., and it is facilitated by a high relative humidity. When spores are deposited on a humid leaf surface they can readily germinate and infect the leaf Fungal growth is optimal between 8 and 20° C. and a relative humidity of ≥80%, and within 6 and 13 days after infection mycelium growth can be observed. Oospores of P. effusa can survive in the soil for up to 3 years, or as mycelium in seeds or living plants.

To date 20 pathogenic races of spinach downy mildew (Pe, formerly known as Pfs) have been officially identified and characterized, and many new candidates are observed in the field. The 20 officially recognized races of Peronospora effusa, are designated Pe:1 to Pe:20 (Pe:1 to Pe:17 were formerly known as Pfs:1 to Pfs17; Irish et al., 2008, Phytopathology 98: 894-900; Plantum NL (Dutch association for breeding, tissue culture, production and trade of seed and young plants) press release, “Benoeming van Pfs: 14, een nieuwe fysio van valse meeldauw in spinazie”, Sep. 19, 2012; Report Jim Correl (Univ. Arkansas) and Steven Koike (UC Cooperative Extension, Monterey County), “Race Pfs: 14—Another new race of the spinach downy mildew pathogen”, Sep. 18, 2012; Plantum NL press release, “Denomination of Pfs: 15, a new race of downy mildew in spinach”, Sep. 2, 2014; Plantum NL press release, “Denomination of Pfs: 16, a new race of downy mildew in spinach, Mar. 15, 2016; Plantum NL press release, Denomination of Pfs: 17, a new race of downy mildew in spinach”, Apr. 16, 2018; Plantum NL press release, “Denomination of Pe: 18 and 19, two new races of downy mildew in spinach”, Apr. 15, 2021; Plantum NL press release, “Denomination of Pe: 20, a new race of downy mildew in spinach”, May 13, 2024).

The officially recognized Pe races are publicly available from Naktuinbouw, Sotaweg 22, 2371 GD Roelofarendsveen, the Netherlands, or via prof Jim Correll (University of Arkansas, cppsi.ucdavis.edu/sites/g/files/dgvnsk8206/files/inline-files/Spinach_downy_mildew_Differentials_ver %20June %202021.pdf).

In recent years various alleles of resistance genes or R-genes have been identified that provide spinach plants with a resistance against downy mildew, as described in e.g. US2013230635, WO2018060474, and EP2848114. Co-evolution of plant and pathogen has led to an arms race in which a R-gene mediated resistance is sometimes overcome as a consequence of the capability of the pathogen to interact with and modify alternative host targets or the same targets in a different way, such that the recognition is lost and infection can be established successfully resulting in disease. To re-establish resistance in a plant, a new R-gene has to be introduced which is able to recognize the mode of action of an alternative pathogenicity factor.

Since R-gene mediated resistance provides a resistance of relatively low durability and the downy mildew pathogen is evolving and adapting rapidly to these R-genes, there is a need in the art to have resistance sources available which are more durable compared to R-gene mediated resistance.

Besides an R-gene mediated defense mechanism, a plant can exhibit a more basal form of resistance which prevents the pathogen from infecting the plant. This non-R-gene mediated form of resistance can be considered as an extremely successful form of defense which in fact is effective for most plant pathogen interactions. Since such a resistance is not based on the kind of interaction and recognition between host and pathogen as is known for R-genes, this defense mechanism is much more durable and provides resistance against a broader spectrum of pathogen races than would normally be expected with R-genes.

Citation or identification of any document in this application is not an admission that such document is available as prior art to the present invention.

### SUMMARY OF THE INVENTION

Given the significant advantages of a non-R-gene mediated resistance, it is the object of the present invention to provide a spinach plant resistant to downy mildew, wherein the resistance is caused by a non-R-gene mediated resistance conferring nucleic acid molecule.

In the research that led to the present invention, spinach plants were developed which have in their genome a locus capable of conferring non-R-gene mediated resistance against downy mildew when present homozygously. The locus of the invention providing broad spectrum resistance is named the p10-locus. Through QTL mapping it was initially determined that the p10-locus is located on chromosome 1 (see US20170327839), which after the publication of the first spinach genome was renamed to chromosome 3. The p10-locus of this invention is thus located on chromosome 3, formerly known as chromosome 1.

Further research using fine-mapping, genome analysis and recombinant analysis has shown the p10-locus comprises two genes that are involved in conferring non-R-gene mediated resistance: a p10 NLR-kinase gene having a coding sequence according to SEQ ID NO: 1 and which encodes a protein having a an amino acid sequence according to SEQ ID NO: 3, and a p10-kinase gene having a coding sequence according to SEQ ID NO: 2 and which encodes a protein having an amino acid sequence according to SEQ ID NO: 4. The naming of the genes is based on an internal denomination.

Thus, the present invention provides a nucleic acid molecule that confers non-R-gene mediated broad spectrum resistance to downy mildew when homozygously present in a spinach plant. This nucleic acid molecule is herein interchangeably referred to as “p10-locus”, “resistance conferring nucleic acid molecule”, “resistance conferring nucleic acid molecule of the invention”, or “nucleic acid molecule of the invention”.

The broad spectrum resistance conferred by the nucleic acid molecule of the invention is defined as providing at least intermediate resistance to the following races of downy mildew (Peronospora effusa): Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

Spinach plants of the invention, carrying the nucleic acid molecule of the invention, can be crossed with other spinach plants comprising one or more dominant R-genes. In this way an even stronger resistance is obtained and the emergence of new resistance breaking downy mildew strains is slowed down thereby increasing the durability of dominant R-genes. The invention thus relates to a spinach plant exhibiting complete resistance to Peronospora effusa races Pe:1 to Pe:20. Such spinach plants suitably comprise the nucleic acid molecule of the invention and one or more R-genes.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### Deposits

The Deposit with NCIMB Ltd, Ferguson Building, Craibstone Estate, Bucksburn, Aberdeen AB21 9YA, UK, on Feb. 24, 2016, under deposit Accession number NCIMB 42554 was made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary, including if the deposit ever become unviable, during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 is a table showing the resistance of spinach plants of the p10-line and the differential set to each of the officially recognized Peronospora effusa races.

FIG. 2 is a table showing the results of the bioassays for two Peronospora effusa races (Pe: 7 and Pe: 17), and the conclusions on downy mildew resistance from all bioassays performed in Example 2.

FIG. 3 shows the alignment of the protein sequences encoded by the downy mildew resistance conferring p10-kinase gene as found in plants of the p10-line (representative seeds having been deposited under deposit number NCIMB 42554) and the wildtype not downy mildew resistance conferring p10-kinase gene as present in plants of spinach cultivar Sp75.

### DETAILED DESCRIPTION OF THE INVENTION

The present invention thus relates to a Spinacia oleracea plant comprising a nucleic acid molecule which when homozygously present provides at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, wherein said nucleic acid molecule comprises:
a) a gene which comprises a coding sequence according to SEQ ID NO: 1 and a gene which comprises a coding sequence according to SEQ ID NO: 2, or
b) a gene which comprises a coding sequence that in order of increased preference has at least 95.0%, 96.0%, 97.0%, 98.0%, 99.0%, 99.5% sequence identity to SEQ ID NO: 7 and comprises an insertion of sequence motif GAAATA between nucleotides 1235 and 1236 of SEQ ID NO: 7 and a gene which comprises a coding sequence that in order of increased preference has at least 95.0%, 96.0%, 97.0%, 98.0%, 99.0%, 100% sequence identity to SEQ ID NO: 8 and lacks a sequence according to SEQ ID NO: 14, wherein the percentage sequence identity is calculated over the part of SEQ ID NO: 8 without the lacking sequence, or
c) a gene which comprises a coding sequence that in order of increased preference has at least 95.0%, 96.0%, 97.0%, 98.0%, 99.0%, 99.5% sequence identity to SEQ ID NO: 1 and comprises an insertion of sequence motif GAAATA between nucleotides 1235 and 1236 of SEQ ID NO: 7 and a gene which comprises a coding sequence that in order of increased preference has at least 95.0%, 96.0%, 97.0%, 98.0%, 99.0%, 100% sequence identity to SEQ ID NO: 2, or
d) a gene which comprises a genomic sequence that in order of increased preference has at least 95.0%, 96.0%, 97.0%, 98.0%, 99.0%, 99.5% sequence identity to SEQ ID NO: 11 and comprises an insertion of sequence motif GAAATA between nucleotides 5940 and 5941 of SEQ ID NO: 11 and a gene which comprises a genomic sequence that in order of increased preference has at least 95.0%, 96.0%, 97.0%, 98.0%, 99.0%, 100% sequence identity to SEQ ID NO: 6, or
e) a gene encoding a protein having an amino acid sequence according to SEQ ID NO: 3, and a gene encoding a protein having an amino acid sequence according to SEQ ID NO: 4, or
f) a gene encoding a protein having an amino acid sequence that in order of increased preference has at least 95.0%, 96.0%, 97.0%, 98.0%, 99.0%, 99.5% sequence identity to SEQ ID NO: 9 and comprises an insertion of amino acids IE between amino acids 411 and 412 of SEQ ID NO: 9 and a gene encoding a protein having an amino acid sequence that in order of increased preference has at least 95.0%, 96.0%, 97.0%, 98.0%, 99.0%, 100% sequence identity to SEQ ID NO: 4, or
g) a gene which comprises a coding sequence that in order of increased preference has at least 95.0%, 96.0%, 97.0%, 98.0%, 99.0%, 99.5% sequence identity to SEQ ID NO: 7 and comprises an insertion of sequence motif GAAATA between nucleotides 1235 and 1236 of SEQ ID NO: 7 and a gene which comprises a coding sequence according to SEQ ID NO: 2, or
h) a gene which comprises a coding sequence according to SEQ ID NO: 1 and a gene which comprises a coding sequence that in order of increased preference has at least 95.0%, 96.0%, 97.0%, 98.0%, 99.0%, 100% sequence identity to SEQ ID NO: 8 and lacks a sequence according to SEQ ID NO: 14, wherein the percentage sequence identity is calculated over the part of SEQ ID NO: 8 without the lacking sequence, or
i) a gene which comprises a coding sequence according to SEQ ID NO: 1 and a gene which comprises a coding sequence that in order of increased preference has at least 95.0%, 96.0%, 97.0%, 98.0%, 99.0%, 100% sequence identity to SEQ ID NO: 8 and lacks a sequence according to SEQ ID NO: 14 and lacks a sequence according to SEQ ID NO: 15, wherein the percentage sequence identity is calculated over the part of SEQ ID NO: 8 without the lacking sequences, or
j) a gene which comprises a genomic sequence according to SEQ ID NO: 5 and a gene which comprises a genomic sequence according to SEQ ID NO: 6. Preferably, this Spinacia oleracea plant of the invention is an agronomically elite Spinacia oleracea plant.

In one embodiment, the Spinacia oleracea plant of the invention comprises a nucleic acid molecule which when homozygously present provides at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, wherein said nucleic acid molecule comprises:
a) a gene which comprises a coding sequence according to SEQ ID NO: 1 and a gene which comprises a coding sequence according to SEQ ID NO: 2, or
b) a gene which comprises a coding sequence having at least 95.0% sequence identity to SEQ ID NO: 7 and comprises an insertion of sequence motif GAAATA between nucleotides 1235 and 1236 of SEQ ID NO: 7 and a gene which comprises a coding sequence having at least 95.0%, sequence identity to SEQ ID NO: 8 and lacks a sequence according to SEQ ID NO: 14, wherein the percentage sequence identity is calculated over the part of SEQ ID NO: 8 without the lacking sequence, or
c) a gene which comprises a coding sequence having at least 95.0% sequence identity to SEQ ID NO: 1 and comprises an insertion of sequence motif GAAATA between nucleotides 1235 and 1236 of SEQ ID NO: 7 and a gene which comprises a coding sequence having at least 95.0% sequence identity to SEQ ID NO: 2, or
d) a gene which comprises a genomic sequence having at least 95.0% sequence identity to SEQ ID NO: 11 and comprises an insertion of sequence motif GAAATA between nucleotides 5940 and 5941 of SEQ ID NO: 11 and a gene which comprises a genomic sequence having at least 95.0% sequence identity to SEQ ID NO: 6, or
e) a gene encoding a protein having an amino acid sequence according to SEQ ID NO: 3, and a gene encoding a protein having an amino acid sequence according to SEQ ID NO: 4, or
f) a gene encoding a protein having an amino acid sequence having at least 95.0% sequence identity to SEQ ID NO: 9 and comprises an insertion of amino acids IE between amino acids 411 and 412 of SEQ ID NO: 9 and a gene encoding a protein having an amino acid sequence having at least 95.0% sequence identity to SEQ ID NO: 4. Preferably, this Spinacia oleracea plant of the invention is an agronomically elite Spinacia oleracea plant.

In one embodiment the Spinacia oleracea plant of the invention comprises a nucleic acid molecule which when homozygously present provides at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, wherein said nucleic acid molecule comprises:
a) a gene which comprises a coding sequence according to SEQ ID NO: 1 and a gene which comprises a coding sequence according to SEQ ID NO: 2, or
b) a gene encoding a protein having an amino acid sequence according to SEQ ID NO: 3, and a gene encoding a protein having an amino acid sequence according to SEQ ID NO: 4, or
c) a gene which comprises a genomic sequence according to SEQ ID NO: 5 and a gene which comprises a genomic sequence according to SEQ ID NO: 6. Preferably, this Spinacia oleracea plant of the invention is an agronomically elite Spinacia oleracea plant.

In one aspect of the invention, the nucleic acid molecule is as present in the genome of a Spinacia oleracea plant, representative seed of which was deposited with the NCIMB under accession number NCIMB 42554.

The invention also relates to a Spinacia oleracea plant grown from the seed deposited under accession number NCIMB 42554.

The invention further relates to a Spinacia oleracea plant comprising the nucleic acid molecule of the invention, wherein the nucleic acid molecule is introgressed from a plant, which is grown from seed deposited with the NCIMB under accession number 42554 or a progeny plant of seed of NCIMB accession 42554 that has retained the nucleic acid molecule.

The invention also relates to a Spinacia oleracea plant comprising the nucleic acid molecule of the invention, wherein the nucleic molecule that provides resistance is comprised in the p10-locus as found in a plant grown from seed of which a representative sample was deposited with the NCIMB under accession number 42554.

In contrast to a resistance mediated by a dominant R-gene, the nucleic acid molecule of the invention only provides resistance when homozygously present. Therefore, the resistance conferred by the nucleic acid molecule of the invention is transferred in a pattern that fits a recessive inheritance. Due to its inheritance, the p10 resistance is considered to be a non-R-gene mediated resistance. Furthermore, due to the resistance profile provided by the nucleic acid molecule of the invention, the resistance is regarded to be a broad spectrum resistance.

Therefore, in a particular embodiment the nucleic acid molecule of the invention is homozygously present, thus providing the spinach plant of the invention with at least an intermediate resistance to the following races of downy mildew (Peronospora effusa) Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

In the context of this invention, intermediate resistance is defined as a plant showing only symptoms of chlorosis, or sporulation occurring only on the tips of the cotyledons in the differential seedling test as described herein.

Furthermore, in the context of this invention complete resistance is defined as a plant showing no symptoms in the seedling test as described herein. Complete resistance is also referred to as full resistance.

The resistance phenotype of plants comprising the nucleic acid molecule of this invention homozygously is assessable in a bioassay such as the seedling test described herein, as illustrated by the examples.

A seedling test is defined as a test wherein spinach plants are planted in trays containing growth medium and optionally fertilized twice a week after seedling emergence. Plants are inoculated at the first true leaf stage with a sporangial suspension having a concentration of approximately 2.5×10⁵/ml of one of the pathogenic races of Peronospora effusa or isolates to be tested. The inoculated plants are placed in a dew chamber at 18° C. with 100% relative humidity for a 24 h period and then moved to a growth chamber at 18° C. with a 12 h photoperiod for 6 days. After 6 days, the plants are returned to the dew chamber for 24 h to induce sporulation, and subsequently scored for a disease reaction. Preferably, 30 plants per race are tested.

In one embodiment, the plant of the invention, i.e. the plant comprising the nucleic acid molecule of the invention, is an agronomically elite spinach plant.

In the context of this invention an agronomically elite spinach plant is a plant having a genotype that as a result of directed crossing and selection by human intervention results into an accumulation of distinguishable and desirable agronomic traits which allow a producer to harvest a product of commercial significance.

In the course of breeding a new spinach plant carrying the broad spectrum resistance locus of the invention, desirable agronomic traits may be introduced into said spinach plant independently of the nucleic acid molecule of the invention. As used herein, “desirable traits” include but are not limited to e.g. improved yield, leaf shape, leaf size, leaf number, leaf color, seed number, seed size, plant vigor, plant height, bolting speed, and resistance to one or more diseases or disease causing organisms. Any one of these desirable traits may be combined with the nucleic acid molecule of the invention.

In a further embodiment the spinach plant of the invention may exhibit complete resistance against one or more of the following Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20 due to the presence of one or more dominant R-genes (e.g. Feng et al. Identification of new races and deviating strains of the spinach downy mildew pathogen Peronospora farinosa f.sp. spinaciae 2014 Plant Disease 98(1): 145-152, WO2018060474, WO2024008703). The spinach plant may further be resistant or tolerant to one of the following diseases: CMV, Stemphylium vesicarium, Colletotrichum, Cladosporium, and Fusarium (see e.g. WO2019063839, WO2024008703). Next to any of the aforementioned resistances the spinach plant of the invention may comprise a delayed or enhanced bolting phenotype. The spinach plant of the invention may also comprise one of the following leaf shapes: savoy, semi-savoy or smooth leaves. The leaves of the plant may independently of their shape comprise elevated concentrations of the red pigment betacyanin in the petiole, veins and/or between the veins of the leaves (see e.g. US20140272083). Next to the aforementioned traits a spinach plant of the invention may comprise a multileaf characteristic (see e.g. US20120054894). For example, the invention in one embodiment relates to an agronomically hybrid spinach variety that is resistant to Peronospora effusa races Pe:1-13 and Pe:15 and intermediately resistant to Pe:14 and Pe:16 due to the presence of two R-genes and the nucleic acid molecule of this invention, respectively, and further resistant to CMV, showing slow bolting and having smooth leaves. Another example of such an agronomically elite hybrid variety is a variety resistant to Peronospora effusa races Pe:1-9 and Pe:11-16 and intermediately resistant to Pe:10 due to the presence of two R-genes and the nucleic acid molecule of this invention, having a fast growing phenotype, but relatively slow bolting, and well suited for the industrial harvest segment.

In one embodiment the agronomically elite spinach plant of the invention comprises the nucleic acid molecule of the invention and a nucleotide sequence comprising a coding sequence having a sequence according to SEQ ID NO: 26 (alpha-WOLF 29).

In another embodiment the agronomically elite spinach plant of the invention comprises the nucleic acid molecule of the invention and a nucleotide sequence comprising a coding sequence having a sequence according to SEQ ID NO: 27 (alpha-WOLF 30).

In another embodiment the agronomically elite spinach plant of the invention comprises the nucleic acid molecule of the invention and a nucleotide sequence comprising a coding sequence having a sequence according to SEQ ID NO: 28 (alpha-WOLF 24).

In yet a further embodiment the agronomically elite spinach plant of the invention is an inbred line or a hybrid.

As used herein, a plant of an inbred line is a plant of a population of plants that is the result of three or more rounds of selfing, or backcrossing; or which plant is a double haploid. An inbred line may e.g. be a parent line used for the production of a commercial hybrid.

As used herein, a hybrid plant is a plant which is the result of a cross between two different plants having different genotypes. More in particular, a hybrid plant is the result of a cross between plants of two different inbred lines, such a hybrid plant may e.g. be a plant of a commercial F₁ hybrid variety.

In one embodiment the plant comprising the nucleic acid molecule of the invention is an F₁ hybrid variety.

Furthermore, the invention relates to hybrid seed that may be grown into a spinach plant comprising the nucleic acid molecule of the invention and to a method for producing such hybrid seed comprising crossing a first parent spinach plant with a second parent spinach plant and harvesting the resultant hybrid seed, wherein the first parent plant and/or the second parent plant comprises the nucleic acid molecule of the invention homozygously, and growing said hybrid seeds into hybrid spinach plants comprising the nucleic acid molecule of the invention either heterozygously or homozygously.

The invention further relates to a cell of a spinach plant of the invention, which cell comprises the nucleic acid molecule of the invention. Such a cell may either be in isolated form, or a part of the complete plant or parts thereof and still forms a cell if the invention because said cell comprises the nucleic acid molecule of the invention. Each cell of the plant of the invention carries the nucleic acid molecule of the invention. The cell of the invention may also be a regenerable cell that can regenerate into a new plant of the invention.

The invention also relates to a Spinacia oleracea seed comprising the nucleic acid molecule of the invention in its genome, wherein a plant grown from the seed is a plant of the invention.

The invention also relates to propagation material suitable for producing a plant of the invention, wherein the propagation material is suitable for sexual reproduction, and is in particular selected from the group comprising a microspore, pollen, an ovary, an ovule, an embryo sac, and an egg cell; or is suitable for vegetative reproduction, and is in particular selected from the group comprising a cutting, a root, a stem, a cell, and a protoplast; or is suitable for tissue culture of regenerable cells, and is in particular selected from the group comprising a leaf, pollen, an embryo, a cotyledon, a hypocotyl, a meristematic cell, a root, a root tip, an anther, a flower, a seed, and a stem; wherein the plant developed from the propagation material comprises the nucleic acid molecule of the invention.

The invention further relates to progeny of a plant, a cell, a tissue or a seed of the invention, which progeny comprises the nucleic acid molecule of the invention as defined herein, the homozygous presence of which nucleic acid molecule confers at least an intermediate resistance to the following races of downy mildew (Peronospora effusa) Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

As used herein, “progeny” is intended to mean the first and all further descendants, such as an F1, F2, or further generation, from a cross with a plant of the invention, wherein a cross comprises a cross with itself or a cross with another plant, and wherein a descendant that is determined to be progeny comprises the nucleic acid molecule of the invention as defined herein. The plant of the invention that is used in this cross is optionally a plant grown from seed of deposit NCIMB 42554, or from progeny seed thereof which is a direct or further descendant through crossing a plant grown from the deposited seed with itself or with another plant for one or more subsequent generations, wherein the progeny seed has retained the nucleic acid molecule of the invention.

This invention also relates to a method for selecting a Spinacia oleracea plant that is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising identifying the presence of the nucleic acid molecule of the invention and selecting the Spinacia oleracea plant homozygously comprising said nucleic acid molecule. In one embodiment the nucleic acid molecule comprises SEQ ID NO: 1 and SEQ ID NO: 2. In one embodiment, the presence of the nucleic acid molecule is identified by using markers SEQ ID NO: 18 and/or SEQ ID NO: 20.

The nucleic acid molecule of the invention comprises two genes: the p10-kinase gene and the p10 NLR-kinase gene.

The coding sequence of the allele of the p10-kinase gene as found in the p10-line (NCIMB 42554) is provided as SEQ ID NO: 2, while the genomic sequence of the p10-kinase gene, including 2 kb upstream and 2 kb downstream, as present in the p10-line is provided as SEQ ID NO: 6. The coding sequence of the wildtype allele of the p10-kinase gene, not conferring downy mildew resistance, as e.g. found in Chinese spinach cultivar Sp75 (Xu et al. Nature Communications 8:15275, 2017) is provided as SEQ ID NO: 8, while the genomic sequence, including 2 kb upstream and 2 kb downstream, of the p10-kinase gene as found in cultivar Sp75 is provided as SEQ ID NO: 12. The p10-kinase gene as found in the p10-line differs from the p10-kinase gene as found in cultivar Sp75 by two deletions: (1) a 558 bp deletion (SEQ ID NO: 13) starting upstream of the start codon and deleting the first 288 bp of the coding sequence (SEQ ID NO: 14) and leading to a new start codon 318 bp after the start codon as found in the p10-kinase gene of Sp75 and (2) a 45 bp deletion within the coding sequence of the p10-kinase gene (SEQ ID NO: 15).

The p10-kinase gene encodes a protein that is predicted to be a cysteine-rich receptor-like protein kinase. The wildtype allele of the p10-kinase gene, not conferring downy mildew resistance, as e.g. found in Chinese spinach cultivar Sp75, encodes a protein (SEQ ID NO: 10) of 317 amino acids long, which is predicted to comprise a phosphorylase kinase domain at amino acid position 13-112 and a phosphotransferase domain at amino acid position 116-312. The p10-kinase gene conferring downy mildew resistance as in a plant comprising the nucleic acid molecule of the invention, such as a plant grown from seed deposited with the NCIMB under deposit number NCIMB 42554, encodes a protein (SEQ ID NO: 4) of 196 amino acids long. This protein is predicted to comprise a phosphotransferase domain at amino acid position 10-191. It lacks the phosphorylase kinase domain that is predicted to be present in the wildtype, not downy mildew resistance conferring, allele as present in for example spinach cultivar Sp75. At the N-terminus of the protein 106 amino acids (SEQ ID NO: 16) have been deleted in the resistance conferring p10-kinase protein. Additionally, the resistance conferring p10-kinase protein has a 15 amino acid deletion (SEQ ID NO: 17) and an S to P amino acid substitution at position 79 of SEQ ID NO: 4, when compared to the wildtype p10-kinase protein as present in Sp75.

The coding sequence of the allele of the p10 NLR-kinase gene as found in the p10-line (NCIMB 42554) is provided as SEQ ID NO: 1, while the genomic sequence of the p10 NLR-kinase gene, including 2 kb upstream and 2 kb downstream, as present in the p10-line is provided as SEQ ID NO: 5. The coding sequence of the wildtype allele of the p10 NLR-kinase gene, not conferring downy mildew resistance, as e.g. found in Chinese spinach cultivar Sp75 is provided as SEQ ID NO: 7, while the genomic sequence, including 2 kb upstream and 2 kb downstream, of the p10 NLR-kinase gene as found in cultivar Sp75 is provided as SEQ ID NO: 11. The p10 NLR-kinase gene as found in the p10-line (NCIMB 42554) differs from the p10 NLR-kinase gene as found in cultivar Sp75 by a 6 bp insertion in the coding sequence.

The p10 NLR-kinase gene encodes a Nucleotide-binding Leucine-rich Repeat protein with an integrated kinase domain (NLR-kinase). The wildtype allele of the p10 NLR-kinase gene, not conferring downy mildew resistance, as e.g. found in Chinese spinach cultivar Sp75, encodes a protein (SEQ ID NO: 3) of 1454 amino acids long, which is predicted to comprise a phosphorylase kinase domain at amino acid position 16-130, a phosphotransferase domain at amino acid position 133-351, an NB-ARC domain at position 576-731 and a Leucine-rich repeat region at position 936-1411. The p10 NLR-kinase gene conferring downy mildew resistance as in a plant comprising the nucleic acid molecule of the invention, such as a plant grown from seed deposited with the NCIMB under deposit number NCIMB 42554, encodes a protein (SEQ ID NO: 9) of 1456 amino acids long. The only difference in sequence between the wildtype allele of the p10 NLR-kinase protein as present in Sp75 and the allele as present in the p10 line is a 2 amino acid insertion at amino acid position 412-413 of SEQ ID NO: 9, which is in between the integrated kinase domain and the NB-ARC domain. The 2 amino acid insertion is necessary for the p10 NLR-kinase protein to be able to contribute to the broad-spectrum downy mildew resistance of this invention.

The invention relates to the use of the polynucleotide sequence of the nucleic acid molecule of the invention or part thereof as a marker for identifying and/or selecting a Spinacia oleracea plant that is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, wherein the polynucleotide sequence comprises polymorphisms compared to a plant susceptible to downy mildew and lacking the nucleic acid molecule of the invention.

A marker can be defined as a reference sequence that comprises the modification(s) that can be detected using any suitable method known. The term “marker”, “molecular marker”, “genetic marker” or “DNA marker” refers to a feature of an organism's genome (e.g. a nucleotide or a polynucleotide sequence that is present in an organism's genome) that is associated with one or more loci of interest. In some embodiments, a genetic marker is polymorphic in a population of interest. Genetic markers include, for example, single nucleotide polymorphisms (SNPs), indels (i.e. insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic markers can, for example, be used to locate genetic loci containing alleles on a chromosome that contribute to variability of phenotypic traits. The term “marker” or “genetic marker” can also refer to a polynucleotide sequence complementary to a genomic sequence, such as a sequence of a nucleic acid used as a probe. The term “marker” then refers to a physical entity that can be used in molecular biological techniques for detecting the mutation.

Methods for detecting markers and specific alleles are abundantly known in the field.

In general, these methods allow to distinguish between two different alleles of a marker, or the presence or absence of a marker on a specific chromosome. Detection of a polymorphism can be achieved by electrophoretic techniques, but the widespread availability of DNA sequencing often makes it easier to simply sequence amplified products directly. Once the polymorphic sequence difference is known, rapid assays for the detection of a polymorphism can be designed for progeny testing, generally involving some version of PCR amplification of specific alleles.

Marker SORZ01 was designed on the 6 bp insertion present in the allele of the p10 NLR-kinase gene as found in the p10-line (NCEVIB 42554) that is absent in susceptible material such as Viroflay and Sp75. Marker SORZ02 was developed on the 45 bp deletion present in the coding sequence of the allele of the p10-kinase gene as present in the p10-line (NCIMB 42554). SEQ ID NO: 18 and SEQ ID NO: 20 represent the alleles of markers SORZ01 and SORZ02, respectively, that indicate the presence of the nucleic acid molecule of the invention. Therefore, the homozygous presence of SEQ ID NO:18 and SEQ ID NO: 20 is linked to the resistance conferred by the p10 locus, which is an at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20. The sequences of SEQ ID NO: 19 and SEQ ID NO: 21 represent the wildtype alleles for markers SORZ01 and SORZ02, respectively, as present in spinach plants not comprising the nucleic acid molecule of the invention.

This invention also relates to a marker for the identification of the nucleic acid molecule of the invention. Preferably, the marker is selected from the group of SEQ ID NO: 18 and SEQ ID NO: 20. Use of marker SEQ ID NO: 18 and SEQ ID NO:20 for the identification of the nucleic acid molecule of the invention is also part of this invention.

The invention also relates to a method for determining the genotype of a Spinacia oleracea plant comprising the nucleic acid molecule of seed deposited with the NCIMB under accession number 42554, comprising detecting in the Spinacia oleracea plant the presence of the nucleic acid molecule of the invention and selecting the Spinacia oleracea plant homozygously comprising said nucleic acid molecule. In one embodiment, wherein the nucleic acid molecule comprises SEQ ID NO: 1 and SEQ ID NO: 2.

This invention further relates to a method for producing an agronomically elite Spinacia oleracea plant comprising at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising:
(a) crossing a first plant selected as comprising the nucleic acid molecule of the invention or a plant determined to have the genotype of the invention as a Spinacia oleracea plant comprising the nucleic acid molecule, with a second plant; and
(b) performing one or more rounds of selfing and/or crossing.

In one embodiment, this method further comprises: (c) selecting after each round of selfing or crossing for a plant that comprises said resistance.

The invention also relates to a method for producing an agronomically Spinacia oleracea plant exhibiting at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising:
a) crossing a plant of the invention with a second parent plant to obtain an F1 population;
b) performing one or more rounds of selfing and/or crossing of the plant resulting from the cross to obtain a further generation population;
c) selecting from among the plants resulting from the further generation population of step
b) a plant that comprises the nucleic acid molecule of claim 1, which plant is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

In one embodiment, selecting a plant comprising the nucleic acid molecule of the invention is done by determining the presence of the nucleic acid molecule of the invention. Determining the presence of the nucleic acid molecule may preferably be done by sequencing or marker analysis.

In another embodiment, a plant comprising the nucleic acid molecule of the invention is phenotypically selected, in particular by determining resistance to pathogenic races of Peronospora effusa as described in Example 1.

In a specific embodiment, the plant of the invention in step a) for producing a Spinacia oleracea plant which is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20 is a plant grown from seed deposited under NCIMB accession number NCIMB 42554, or a progeny plant thereof that has retained the nucleic acid molecule of the invention.

The invention further relates to a hybrid Spinacia oleracea seed and to a method for the production of a hybrid Spinacia oleracea seed comprising crossing a first parent plant with a second parent plant, wherein the first and the second parent plant comprise the nucleic acid molecule of the invention, and wherein the homozygous presence of said nucleic acid molecule leads to at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20 in a plant that is grown from the hybrid seed.

In a particular embodiment, the first and/or the second parent plant is a plant grown from seed deposited under NCIMB accession number NCIMB 42554, or a seed of a progeny plant thereof that has retained the nucleic acid molecule of the invention.

A plant of the invention can be produced by introducing the nucleic acid molecule of the invention from another plant which comprises said nucleic acid molecule by applying commonly used breeding techniques, such as crossing and selection. Such introduction can be from a plant of the same species, that usually can be crossed easily, or from a plant of a related species. Difficulties in crossing can be overcome by applying techniques known in the art such as embryo rescue, cisgenesis, and the use of a bridge species. Suitably the nucleotide sequence of the nucleic acid molecule of the invention or a polymorphic part thereof is used to follow the incorporation of the nucleic acid molecule into another plant.

Alternatively, the nucleic acid molecule of the invention can be used to produce a plant of the invention using a transgenic approach. Techniques that can suitably be used comprise general plant transformation techniques known to the skilled person, for example Agrobacterium-mediated transformation method and protoplast transformation. Genome editing methods such as the use of a CRISPR/Cas system can also be employed to obtain a spinach plant that exhibits at least intermediate resistance to Peronospora effusa Pe: 1 to Pe: 20. The current invention also relates to the use of a plant of the invention as a crop, as a source of seed or as a source of propagation material.

This invention also relates to a method for producing a Spinacia oleracea plant exhibiting at least intermediate resistance to Peronospora effusa, comprising the step of introducing the nucleic acid molecule of the invention, wherein the nucleic acid molecule when homozygously present in a Spinacia oleracea plant confers at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20. In one embodiment the nucleic acid molecule of the invention is introduced by cisgenesis.

The invention also relates to a method for growing a Spinacia oleracea plant at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, which method comprises the step of germinating a seed comprising the nucleic acid molecule of claim 1 in its genome. In a particular embodiment the Spinacia oleracea plant is grown from a seed deposited under NCEVIB accession number NCIMB 42554, or a seed of a progeny plant thereof that has retained the nucleic acid molecule of the invention.

### Sequence Information

The sequences disclosed herein are provided in the following Table 1:

TABLE 1
Sequence information.

| | |
|---|---|
| SEQ ID NO: 1: | ATGACTGGTACTTGTTTCGGTGATTTGGGTGGTGTGAGGAGCTGTT |
| coding sequence of | GGGGTTATAAAGTGAAATTGGTGCGTAATCAAGAGGAAATTAAAG |
| p10 NLR-kinase | ACGATACCCTATTAAAAAATGTCAGATTGTTTACCTGGAAAGAGC |
| gene as present in | TGCAGAAGGCAACAGATAACTTTAACTCCAGCTCACTTATAGGCA |
| NCIMB 42554 | AGGGTGGGTTTACATCTTACTATAAGGGCACTTTAGAAGATGGGA |
| | CTGAGGTGGCAGTGCTATCACATAGAAAGGGGGGTGTAAATCAGA |
| | AGCGAGATTCGGATTTTACTTCTTTTATTAATGTGATGTCCAGAAT |
| | CCGGCATCGGAATGTGGTTGAGTTGATCGGTTGCTGTATTGAGGGT |
| | GATCATATGATTTTTGTGTATGAGTTTCTTGGGTATGACAGGCTGG |
| | ATCAGTIGTTAGTTAGTAAGTCTGAGGCGATAGATTGGCCTACTAG |
| | AGCTTCTATATGCATTGGCATTGCTCGTGGTCTTGACTATCTTCAA |
| | GAGGGGGCTAGTCGTCGTCTTGTACATCGTGCTATCAATCCTGATA |
| | ATATATTTCTTGATAGTAACTTCAATCCCAAAATAGCAGGTTTCCG |
| | CATAGCACAGTTGTTTGATGATGATGAATCACAATATAGTGGAGT |
| | ATATACGGCATGCTTACCATCTTCATATTATACGGATCCTCAATAC |
| | TTGGCTACAGGAAGATGTACAGAGAAGGTTGATGTTTATAGCTTT |
| | GGAATTATTTTACTTGAAGTCATCAGTGGCAGGAGAGTTCACGAG |
| | TTTCAGAGAGATGGCAGGAGAGTTCACAAGTTTCAGAGAGATGGC |
| | AGTGGGGTTACGCTGGTGGATTGGATTTTGGAACTACGGAAAACT |
| | GATATGATTCGTGAGATTGTTGATCCAGCTTTATCTGACTTTCCAG |
| | AGGATGAGATGATACGCTTTATTGATGTTGCACTCTCTTGTGTTCA |
| | GTACTCATCAAAGCTCAGACCAAACACGTCGGAGGTTTTGCTTAT |
| | GCTCTTGGGAGGTTATGACTTCAGTAAGAAGGTATTAAGAAATCT |
| | GGAACAATCTTGGCAGTCTGGCTATCATGATGAAGTTGGCAGTCTT |
| | AGTCATGATAAAATTTCTTCAACTCCTGTACCTAGAAGGAAACAA |
| | GATTTTCTAGTGGCAGACACCACGACAAGAAGGATTCCAAGTCAG |
| | CCAGCTACTGAATCAATCAGTAGGACGACACGTGGTTGCGAAATA |
| | GAAATAGAAATAGAGAGTGACCTTGACACACATTCTGGTGCATCA |
| | GAACAAATGGAGATTTCACATGATCTAGAAACTGAATTATACAGA |
| | TCTATTGAGGCAAACATCTCTGCTGCAGCTTCTGTACCTGGACCAG |
| | GTGACAAAAATGCATTGAAACCTGAAGTTGTCAGCCCATCTTCTCC |
| | AAGTACTAACAATGGAAGCCCTTCAACTGTAAGTACTGATATTCA |
| | TGTCCCTGGAGAAGCGTTCGAGACAACAGAGCATTCGCAGATGTC |
| | ACATGATCTAGAAGCTGAATTTCAAATGAGCGAACCATCTTTATC |
| | AGAGTTTCTGTTTGGCCAAATTGCTATGAAAAGTGATGTAAGGGC |
| | TACTTCATCTGTGGAAATTAACAATTGCGTCCAGAATGAAGCAGT |
| | CGACAGGTTTTCATTTCAGGCTGAAATCAGTGGAACAGAGGAGGT |
| | TCTTTCACTACCAAAAGTTAATATGAAGAGATCAATTCATTGTACT |
| | CTAGAGAAGATTTTTGAAGCCTTTCAGGATTCTAATCGCAGAAGA |
| | ATAGGTGTTTATGGAAGAGGGGGTAGTGGCAAAACTACCATGCTA |
| | AAAAACTTGTTCCATCATTCTGCAACAAAGGAGTIGTTTGACCAA |
| | GTCCTTTTTGTGACTGTCCCCAGGTTTGCTCATCAAAAACATGTAC |
| | GAGATGAGATTGTACAACAGTTGTCATTGAATGTACAAGAGTCCT |
| | GCTCAGAGCATGAAATTTCACATCTTATTCATCAGGCTCTAGTAGA |
| | CGGAAAGTTTCTGGTGCTTTTGGATGATGTTTGGGAATCAATTAAC |
| | TTGCATGAAGTTGGAATACCAAATCCTGATCCAGAGAATAGTTGT |
| | TGGTTGGTATTGGCTACTAGGTCTCTGGATATATGCCATGTCATGG |
| | CTGATAGGGAGATAGAAATGGAGGTTCTCTCTGATGCGGAAGCTT |
| | TGGAATTATTTTACTGTCAACTGGGAGATATAATTGATTCTCCAGA |
| | AATTAAGTCCTACGCTCAAGCTATTTCCAAGGAATGCTATGGCTCT |
| | TCAGTGATAATTTCTGCCATTGGAAGTGCCTTAAGAGAGGAAAAC |
| | AGTATTGAACAATGGAAGCTTGCACTGAGAAATCTTCAGTCAGGT |
| | GATACAGCTAGCACAAATGATCATCTTTTTTTTAACCCGCACATAA |
| | AATATTGTTATGATAGATTGAAAGCTCGTGACGTGAAGAATTTCTT |
| | TCTTTACACCGCATTGTTTCAACAAGATAAAGAAATTGATAGTTCC |
| | ATGCTGGTAGATCGCTTCATTAGTGAAGGCTTAGTTGGTAGGTCGA |
| | CAGTGGCTGCTTATAAAAAGGGACATGATATTGTAGTGCGTCTTA |
| | CAAACGCTTCTTTGTTAGAGGCTAATGATGACGGTCTAATGGTTAA |
| | GATGCACAATGTGGTCAGGGATTCAGCATTGGAAATCCTTTCATC |
| | AGGTATAGAAGGTTATCAAGTATTAATGAGATTTCCAGAATCAGA |
| | TAAGGATGAAGATCGATCCTTTCACGAAAGTAGCTGGGCTAACAA |
| | TAACAAGCTGGTTGACAAGTTCGATTCATTCAGCATCCAGATGCCT |
| | AGAATTCACAAATACTTGACTAGAGCTGGTGCTCATTTGAAGGAA |
| | TCTCCGTCACCTGAAGAATGGGAGCAAGCTACTATGATGTTCTTGA |
| | TGGATAATGAGTTGTCAAGTCTTCCAAGCAATCCAAGATGCTATA |
| | ATTTAATAGCATTGTTCCTTCAAAGAAACAGCTCCTTAAGGGTGAT |
| | ACCAACATCCTTTTTTGACAGCATGTCTACTCTTAAAATTCTGAAT |
| | CTCTCCAAAACCAGAATAAAGTCTTTACCTGATTCCTTTTCTAGAC |
| | TCAAAAGCCTCCAAGTCTTACTGCTGCGAGATTGTGAGCGCTTGCT |
| | TGTGCTTCCTTCATCAATTGGATTTCATAAATCTCTGAAGGTGCTT |
| | GATCTTCAAGGTACCGAGGTAACCCACTTGCCTGATAGTATTGGTA |
| | AATTAGAAAGCCTGAAACAGTTGAGAGTTTCATTCTATGGATCCA |
| | TTAATCGTTGTGAATACAATAAGTTGCCTTCAAAGTTGGTATCAGA |
| | TGGAATCATTTCGAGCCTTAAGCTTAGAGAACTTGGTGTTTTTGTT |
| | TATCCTGGAGATCGACGTTGGACATTGAGTGCATCAGGTATCACG |
| | AATGAGATCAGTAGCCTGAAGTIGTATGTGCTTTATTTTCACTTCC |
| | CAAAAGTAGAAAATCTCGAGCACTTTGTGCATACAAGCCAGTCGT |
| | GGAAGGCAAGAGAGTTGGCTAAATTCAATTTTATTGTTGGACATG |
| | ATTTCAAACGTATTGTATCTTTGGTTTCAAATGATGCAGAACTCTT |
| | GTACAACGAAGGTCAAAGATGTTTAAGATTTGTGAATGGTGAAAG |
| | TATACCTGATCCGGTGGTAGAGGTATTGACACGAGCTACTTCTTTT |
| | TACTTAGACCATCACCTGAACATCTCCAATCTATCACAATTTGGAA |
| | TTGACTGCTTTGATGGCTTAAAATTTTGCATACTAAGGGATTGTCC |
| | GGAGCTGGTAGGAATTTTGGATAGTCAAGACCAGCGAAGTGCATT |
| | CCTTCCTTTTCTAGAATACTTGAGTATCAACTACCTCTGGAACTTA |
| | AAAAGAATTTGGGTTGGTTATATTGCAGCAGGAAGTTTTGCTAGG |
| | TTAAGATGCTTATCGATTCATGCTTGTCCAAAATTGAGCTATGTTC |
| | TTACTTGCCCCATGCTTGATATTCTCCGTAATTTGGAGGAGTTGGT |
| | GATTGAAGACTGTGTATCCCTAAAATATGTTGTGAGTGAAGACGA |
| | GGATGTCAAAGAGATCAAGTATGCTGATGTAGACAGTGTTTCTGC |
| | TCTTGAGTTGTCTCCGCTTCATATGTTGAAAGTATTAAAACTTCAT |
| | TATCTACCAGAGATGTGCGGCATTTGGAGAACCCGGTGGCCTCCA |
| | TTGGAGTACGTCAGCTTTTACGATTGTCCCAAGCTCAGAAATCTTC |
| | ATATGGAAGGGTATGATGACACATTCATTAAAGAAATAGCAGCAG |
| | ATAAAGGGTGGTGGGATTCTTTAGAGTGGGACGATCTTGAACTGT |
| | CAAGAAGACTTCTCAAGCACGTGACCGAACTCCATGTTGATGAAT |
| | TGTAA |
| SEQ ID NO: 2: | ATGCTGGTGCACGAGTTTGTAGGGGATTGCAGCCTGGATCAGTTG |
| coding sequence of | TTGTTTCCAGACTCCAGCAGTGGGGTAGGAGATCGTTCTGTGCTAG |
| the p10 kinase gene | ATTGGCCCATGAGAGTGTCCATATGCTTTAAGATTGTTCGGGTTCT |
| as present in | AGAATATCTTCACTGCGGGATCAGTCCCCGTGTTGTGCTTCGTGAT |
| NCIMB 42554 | TTCAAGCCTGGGAATGTGTTAATGGATTCAAGTTTCAACCCCAAA |
| | ATAGTCCCAAGCCGTATTTGTGGAACAATTGGCTACTTGGATATTG |
| | AATATTGTATAACAGGAAGATATTCAGAGAAGACAGATGTGTTTT |
| | CTTTTGGAATTACTCTACTCGAAATCATCAGTGGTAAGAAAAATCT |
| | GCGGTTTATCTTGGTGAATTGGGCTTGGGACCTACAAGCAAGTGG |
| | TAGGATTCCTGAGATTGTCGATCCTACTCTATCTCAAGTTTCAGAA |
| | GATGAAGTGATACGTTTTGTCAAAGTTGCGCTTTCGTGTGTCCAAT |
| | ATTCAGAACTCAGACCGAGCATGTCTGAGGTAAGGGTAATGCTCT |
| | CAGACGGATATGACTTCAGTAACATGGTACTAACTAGACCATAG |
| SEQ ID NO: 3: | MTGTCFGDLGGVRSCWGYKVKLVRNQEEIKDDTLLKNVRLFTWKEL |
| amino acid | QKATDNFNSSSLIGKGGFTSYYKGTLEDGTEVAVLSHRKGGVNQKR |
| sequence of the p10 | DSDFTSFINVMSRIRHRNVVELIGCCIEGDHMIFVYEFLGYDRLDQLL |
| NLR-kinase as | VSKSEAIDWPTRASICIGIARGLDYLQEGASRRLVHRAINPDNIFLDSN |
| present in NCIMB | FNPKIAGFRIAQLFDDDESQYSGVYTACLPSSYYTDPQYLATGRCTEK |
| 42554 | VDVYSFGIILLEVISGRRVHEFQRDGRRVHKFQRDGSGVTLVDWILEL |
| | RKTDMIREIVDPALSDFPEDEMIRFIDVALSCVQYSSKLRPNTSEVLLM |
| | LLGGYDFSKKVLRNLEQSWQSGYHDEVGSLSHDKISSTPVPRRKQDF |
| | LVADTTTRRIPSQPATESISRTTRGCEIEIEIESDLDTHSGASEQMEISHD |
| | LETELYRSIEANISAAASVPGPGDKNALKPEVVSPSSPSTNNGSPSTVS |
| | TDIHVPGEAFETTEHSQMSHDLEAEFQMSEPSLSEFLFGQIAMKSDVR |
| | ATSSVEINNCVQNEAVDRFSFQAEISGTEEVLSLPKVNMKRSIHCTLE |
| | KIFEAFQDSNRRRIGVYGRGGSGKTTMLKNLFHHSATKELFDQVLFV |
| | TVPRFAHQKHVRDEIVQQLSLNVQESCSEHEISHLIHQALVDGKFLVL |
| | LDDVWESINLHEVGIPNPDPENSCWLVLATRSLDICHVMADREIEME |
| | VLSDAEALELFYCQLGDIIDSPEIKSYAQAISKECYGSSVIISAIGSALRE |
| | ENSIEQWKLALRNLQSGDTASTNDHLFFNPHIKYCYDRLKARDVKNF |
| | FLYTALFQQDKEIDSSMLVDRFISEGLVGRSTVAAYKKGHDIVVRLTN |
| | ASLLEANDDGLMVKMHNVVRDSALEILSSGIEGYQVLMRFPESDKDE |
| | DRSFHESSWANNNKLVDKFDSFSIQMPRIHKYLTRAGAHLKESPSPEE |
| | WEQATMMFLMDNELSSLPSNPRCYNLIALFLQRNSSLRVIPTSFFDSM |
| | STLKILNLSKTRIKSLPDSFSRLKSLQVLLLRDCERLLVLPSSIGFHKSL |
| | KVLDLQGTEVTHLPDSIGKLESLKQLRVSFYGSINRCEYNKLPSKLVS |
| | DGIISSLKLRELGVFVYPGDRRWTLSASGITNEISSLKLYVLYFHFPKV |
| | ENLEHFVHTSQSWKARELAKFNFIVGHDFKRIVSLVSNDAELLYNEG |
| | QRCLRFVNGESIPDPVVEVLTRATSFYLDHHLNISNLSQFGIDCFDGLK |
| | FCILRDCPELVGILDSQDQRSAFLPFLEYLSINYLWNLKRIWVGYIAAG |
| | SFARLRCLSIHACPKLSYVLTCPMLDILRNLEELVIEDCVSLKYVVSED |
| | EDVKEIKYADVDSVSALELSPLHMLKVLKLHYLPEMCGIWRTRWPPL |
| | EYVSFYDCPKLRNLHMEGYDDTFIKEIAADKGWWDSLEWDDLELSR |
| | RLLKHVTELHVDEL |
| SEQ ID NO: 4: | MLVHEFVGDCSLDQLLFPDSSSGVGDRSVLDWPMRVSICFKIVRVLE |
| amino acid | YLHCGISPRVVLRDFKPGNVLMDSSFNPKIVPSRICGTIGYLDIEYCITG |
| sequence of the p10 | RYSEKTDVFSFGITLLEIISGKKNLRFILVNWAWDLQASGRIPEIVDPTL |
| kinase as present in | SQVSEDEVIRFVKVALSCVQYSELRPSMSEVRVMLSDGYDFSNMVLT |
| NCIMB 42554 | RP |
| SEQ ID NO: 5: | TACTGAAAGTAATAATTCTGCAATTGAATTGTGGTTAGAAAAGAT |
| genomic sequence | TAAGTAATGCAAGATAGTGTACCTGGAAGGCTGGAAGAAACTACG |
| of the p10 NLR- | GAAGTTAGAAATTGAAATTCAGAACTGAGGAATGAAGAATGAAG |
| kinase gene as | AATGAAGAATGAAGAATTGACTTAACCCAAAAACTGGACTTGCAT |
| present in NCIMB | TCGAGCTAGATTTTAAAATTTGACTTAACCCATTATCTGAGTCTGG |
| 42554, including | TAAGTTGCGTTGGATTCACATTATTTTCATTTATTTTTTCTACGAAT |
| 2 kb upstream and | CTTTTAAAGTTGTCACTTTTGAAATAACTACTCCCTCCGTCCCATA |
| 2 kb downstream | ATTATCTTCTTGTTTGACCAAAAACACGGATTTTAAGAAAAGTGTA |
| | ATATAGTACATGAAAAAGTGGAATAAAGTACAAATGATGATTGAG |
| | TTATATGAAAAAGTGGAATAAAGTACATGTGAAAGAGAATATACT |
| | ACATGGGAAAAGTAGAATATAGTACATGGGTGGGGTTTTCAATTC |
| | AATTTTAATTAATATAGTATATGAGAAAGTGGGAACCTTAGTAGC |
| | CAAAATTAGAAACAGGAAGATAATTTTGGGACGCTCATTTAGGAA |
| | AGCAGGAAGATAATTTTGGGACGGAGGGAGTAGAACCTTATAGGT |
| | TTTTTTTAATATATCCAACTCAAACTTTCTCCGTTAAGAAAATCAC |
| | CGTCCAATTCTAATTCCATCAAAATTATTGTTAGTGGGGTCCACAT |
| | CCAACGACCAAATTATGTTTTTCCTCCATTATATCGTAAAACATAA |
| | ATGATATAATGAGAGGGAGAGGTGAGGAGAGGTTTCTTGTCCGCT |
| | TTCGTTGTGTTTTCATTGTGAAATAATATTAAAACATTTTAAATAT |
| | ACATGTGATTTTACATAGAGTATAATGGTTTTACCCTCGTGCTTCT |
| | TAAGAGTAGGAAGAGCTTGGCCTTGATTCAAATACTATAATGAAG |
| | CTATTACAGTTGATATATTTATTCATACTTGGGTGACAGATAAAAA |
| | TGCTTAATCTATTCCTAAATCTAATAATATGATGAGTTTTCCGATC |
| | ATCAAATAAACAATTGCAATGTATACGTAGTACTCCGTGATACTCC |
| | CTCCATCCCTTAATAATCGCACCGCTTTTCTTTTCGGGCCGTCCCTT |
| | AATACTTGCACCGCTTCTATAAATAGAATTATTCACCAATGTTATA |
| | TTATTTCGCACACTTACCTACTAACCCATTCACACTCCCACCCCCC |
| | ACTTCTCACCCCTTACCCATTTTTCACTAATTATATTAAAAAAATA |
| | CGGAGTACTCCACTATCAAATAACACCTATTAAATTAATAAGTGA |
| | ATTCAAGTGACTTAAATTCCACACCGGTCAAACCAATACGAGTAT |
| | TAAAGGACGAAGGGAGTAGTTATTTTTACACTTCAACCAAAAAAA |
| | ACTTGGAAAACCTGGCTACACTAATTTTGTAAAATCTGAAAAGTC |
| | AAAATAAAAATCACTCACGGTCCACATAGACTGCTGCATCCGTTTT |
| | CCCCCACACAATGCTCCGACATCGGATGATTTCCGGGAAAAAAAA |
| | TCTAATTGTTTTAAGTCAGACTGATTACACAAAAATTGACTTTTGA |
| | CGAGCAATTCTCCAGTCAGTGTGTTACGGTGTCATTGATGCGGAAT |
| | CTCCGGTGCTTTCTTTTATTAATCAAATCTCTACATTTCAATCCAAT |
| | TCCAGGTACATAATCTACTGTTCTTTTCACGCAAATTAGGTCCTAC |
| | ACTCGAGAAGAACATACAATTCATCCTCCATGTTGCATATATAATG |
| | CTCTCGTGTTTACAATCTAACAATTGCTAAATTCATTGCATGAACT |
| | TATTTTGTAATTTAATCAGAATGACTTGTAATTCTTCATTTGTTGTT |
| | GTAATTCCTTTTCAGATGAGTTAATTTCCGTTGAAATTATGTGAAT |
| | TTTGGACAGCCCATCGTCTTTAAACTGCTGAATTTGTCTGAATTTG |
| | ATCTGGGTATTGGAGAAATTGCAGTGATAATCAGAAATGACTGGT |
| | ACTTGTTTCGGTGATTTGGGTGGTGTGAGGAGCTGTTGGGGTTATA |
| | AAGTGAAATTGGTGCGTAATCAAGAGGAAATTAAAGACGATACCC |
| | TATTAAAAAATGTCAGATTGTTTACCTGGAAAGAGCTGCAGAAGG |
| | CAACAGATAACTTTAACTCCAGCTCACTTATAGGCAAGGGTGGGT |
| | TTACATCTTACTATAAGGGCACTTTAGAAGATGGGACTGAGGTGG |
| | CAGTGCTATCACATAGAAAGGGGGGTGTAAATCAGAAGCGAGATT |
| | CGGATTTTACTTCTTTTATTAATGTGATGTCCAGAATCCGGCATCG |
| | GAATGTGGTTGAGTTGATCGGTTGCTGTATTGAGGGTGATCATATG |
| | ATTTTTGTGTATGAGTTTCTTGGGTATGACAGGCTGGATCAGTTGT |
| | TAGTTAGTAAGTCTGAGGCGATAGATTGGCCTACTAGAGCTTCTAT |
| | ATGCATTGGCATTGCTCGTGGTCTTGACTATCTTCAAGAGGGGGCT |
| | AGTCGTCGTCTTGTACATCGTGCTATCAATCCTGATAATATATTTC |
| | TTGATAGTAACTTCAATCCCAAAATAGCAGGTTTCCGCATAGCAC |
| | AGTTGTTTGATGATGATGAATCACAATATAGTGGAGTATATACGG |
| | CATGCTTACCATCTTCATATTATACGGATCCTCAATACTTGGCTAC |
| | AGGAAGATGTACAGAGAAGGTTGATGTTTATAGCTTTGGAATTAT |
| | TTTACTTGAAGTCATCAGTGGCAGGAGAGTTCACGAGTTTCAGAG |
| | AGATGGCAGGAGAGTTCACAAGTTTCAGAGAGATGGCAGTGGGGT |
| | TACGCTGGTGGATTGGATTTTGGAACTACGGAAAACTGATATGAT |
| | TCGTGAGATTGTTGATCCAGCTTTATCTGACTTTCCAGAGGATGAG |
| | ATGATACGCTTTATTGATGTTGCACTCTCTTGTGTTCAGTACTCATC |
| | AAAGCTCAGACCAAACACGTCGGAGGTTTTGCTTATGCTCTTGGG |
| | AGGTTATGACTTCAGTAAGAAGGTATTAAGAAATCTGGAACAATC |
| | TTGGCAGTCTGGCTATCATGATGAAGTTGGCAGTCTTAGTCATGGT |
| | GAGTTATTTATAAAAAAAAAAAAAATCGTTTCGCTTCATGAAATTT |
| | AGATCAATATAAGCTTGCCTTTACCGTAAGTCATGGTTTGATTTCA |
| | AACAATGCCATTTTCTGCTTTTGCTTTTTGTATATGATAACAGCTTG |
| | CTAGGATGACTTCTTGATTGGCTTTAGGAACCATGTCTTCTTTCAC |
| | GTCTGCTTGATGATCTTTGAATGTTATTGCAATCAAGTTTATTGTGT |
| | AATTTGATGAAGTGAAGTAGTAAAACATACATAGTAGTAATTAGT |
| | AAAAACACCTAGCCCATGTTGTTAGATGGGTAGGACATAAATCAT |
| | CATAAGTGCTTCATTTATAATTTGGATTTGTATGAACAAAAGTTAT |
| | TTCTGAAATAATTTCAAATCTTCAATCATCTCATACATATTATTTGT |
| | AGTGAACAATTTTAGAAGTTTCTGAAAAAGTCCTCATTTTTTGTAA |
| | GTCGTGATGATGGAGATTAGTATTCAGGGACTTTGACCATTGAAC |
| | CTATTGATCTCAATGTAGGGTAGATGTCATCAAAGACTGGGTATC |
| | ATAATTTATGAGATGGAATTTTTTTTATTCAAAGACTTCTTCCAAC |
| | TTTTTTTGGTGTCCTTTTCCGCCATTTGGATGGGATGTGGTGTGGG |
| | GTGGGGTGGGGGGGTTATTTTGCTTGAGCTAAGTGGCTAGTTTTGT |
| | AGAAGGCATGTTGACCATAACTAAGGTCAACTTTTTGTTGTAGTTG |
| | GGAAGTTTCTGGAGTATGCTTTTTCAGTATCCAGTAAATAATCCAA |
| | TCTATCTGCTTTTTGGGCATACTCCTGTTGTCCAAGGGAAATGGGG |
| | ACTTATTTTAAATAAGAATAATTGTTATATCGTCATGTTCATTAGT |
| | GAGCAGATAATCTGCAGCAATTGTTCGTTTTGAGGAATGTCCTTTT |
| | TTTGTCTTCCCTTCTTATTTTATTCTTGGTGGACATATCATCTTGAC |
| | TGTTCACACAAAAAACACAAACTACATACTCTAAGTCTACAAATA |
| | ACATGAACCAAAAGGTGTAACTCGTATACAACCCATAAATACACT |
| | ACCAAAATTGCTTTCAGAAACGATATGGAAAAATGGAAAACTGTG |
| | AACATGATATGAACCATAGATTGCTTGGGTTTAACTAGTTTCGCTT |
| | TCAACAAAATGAGAGGACTAGTTTTGTTACTTTTTTAAAGCACTTT |
| | GGACAAGTAATCCCTCTTATATTTGAGTTTATTGATCGTTTGATTTC |
| | TGAATGCAGTATGATAAGCTGCACAGTTTTCTAAGTGCTTGTTCTT |
| | TTAAATGGAGTAAATTTTTGAAATATCATTATGTGTTCTCCTTAAC |
| | ACCACCAAAACAAAATTACATGCTAAAGAAAGTATAAGTGGAAAT |
| | TGACAACATATATTGGCTATCGGAAACTAATTGCACTTGAAATCAT |
| | TCACTAGGATCTTCTTTCAGTGATCATTTTTAAGGAATATGGTCTA |
| | AACTCTGAGTCTTTACTTTATCCCTATGTCGATATAAAGATAAAAA |
| | TACAACGGATTATAACTCAAACTACTATACAAGTATACATGTATA |
| | AGTAGCTACATATAATGTGACACTTCATCAATGACTACTGTGGTAT |
| | GTGCAAACAATGAATGACACAGTACTCATCAGACATTTAAATTCC |
| | ATATTCTCCTATTTCAAGTACTCCCTCCGTCCCATAATTATAGTCCC |
| | GTTTGACCAAAAACACGAATTTTAAGAAAAATGGAATATAGTACA |
| | TAAAAAAGTATAATAACGTACAAATGATGATTGGTTTGTATGGAA |
| | AAGTGTAATAAAGTATGAGAAAGAAAAATATAGTACAAGGGAAA |
| | GTGGAATATAGTACTTATTTTGTTTTGTTGTGTTTTCAATGCATTTT |
| | TTAATTAATATGGTACATGAGAAAGTGGGGACCTTAATAGACAAA |
| | AATAGAAACAGGATTATAATTTTGGGACGCCCGATTTAGAAAACA |
| | GGACTATAATTTTGGGACGGAGGGAGTAATCCATAAGGAAATGCA |
| | TGCATCAATTACAACTTACAACTCTTACCTTCAGCCAATTAGAAAT |
| | CAGCTTTCCAGATCTAATTAGCAAAAAACAAGTGGGATTGTGTCA |
| | TAGCTTAACTGAAAAATCAGATTTGTTGCTTAATAGCTTAACTGAA |
| | AAAAATCAGATTTTTACTCTGGTGTCTGTTCAGGTGGTCCTTTGTC |
| | TTTTGATTTGATGTGGGACAATGTCACCTTTTTTGGCGGCATTATG |
| | GGCTAATGCGGCGGGGACTTTTGGGAGGTTCGAAGGAAATGGCTT |
| | AATCTATAACTTGCCTTTACAGAAGAACTTGTTCTCTTATGTTTAT |
| | ACTCCTTTTTCAAAGGTGTTACATAATACTTTCTTTTTTTATATATA |
| | TAAAATGAACATTTATATGAAGGAATGGTAGTAAGTCTAGTAACC |
| | TGCCTATGCTGCTATGCATGATGCAACTGTTGCTAATTAAATCGTA |
| | CCAAAATCTGTTATTATCAAGTTTTGAGTATATAAACATTCATATA |
| | GCTCGTGGTTAAAAGCCATTTTGTCCCAAAGGGACTGAGTCTCTGG |
| | ATATGGCTTTGGTTTGACGACCACAATAGTGTTGTTATGCTGAAAT |
| | TGCTGTTTGGTATTGAGGCTCATTTATATCTCTGGATTTTGGGACA |
| | TACAGTTAATTTCTTATTCTGTTTTCCTAATATGGCTACTTTTACAG |
| | ATAAAATTTCTTCAACTCCTGTACCTAGAAGGAAACAAGATTTTCT |
| | AGTGGCAGACACCACGACAAGAAGGATTCCAAGTCAGCCAGCTAC |
| | TGAATCAATCAGTAGGACGACACGTGGTTGCGAAATAGAAATAGA |
| | AATAGAGAGTGACCTTGACACACATTCTGGTGCATCAGAACAAAT |
| | GGAGATTTCACATGATCTAGAAACTGAATTATACAGATCTATTGA |
| | GGCAAACATCTCTGCTGCAGCTTCTGTACCTGGACCAGGTGACAA |
| | AAATGCATTGAAACCTGAAGTTGTCAGCCCATCTTCTCCAAGTACT |
| | AACAATGGAAGCCCTTCAACTGTAAGTACTGATATTCATGTCCCTG |
| | GAGAAGCGTTCGAGACAACAGAGCATTCGCAGATGTCACATGATC |
| | TAGAAGCTGAATTTCAAATGAGCGAACCATCTTTATCAGAGTTTCT |
| | GTTTGGCCAAATTGCTATGAAAAGTGATGTAAGGGCTACTTCATCT |
| | GTGGAAATTAACAATTGCGTCCAGAATGAAGCAGTCGACAGGTTT |
| | TCATTTCAGGCTGAAATCAGTGGAACAGAGGAGGTTCTTTCACTA |
| | CCAAAAGTTAATATGAAGAGATCAATTCATTGTACTCTAGAGAAG |
| | ATTTTTGAAGCCTTTCAGGATTCTAATCGCAGAAGAATAGGTGTTT |
| | ATGGAAGAGGGGGTAGTGGCAAAACTACCATGCTAAAAAACTTGT |
| | TCCATCATTCTGCAACAAAGGAGTTGTTTGACCAAGTCCTTTTTGT |
| | GACTGTCCCCAGGTTTGCTCATCAAAAACATGTACGAGATGAGAT |
| | TGTACAACAGTTGTCATTGAATGTACAAGAGTCCTGCTCAGAGCA |
| | TGAAATTTCACATCTTATTCATCAGGCTCTAGTAGACGGAAAGTTT |
| | CTGGTGCTTTTGGATGATGTTTGGGAATCAATTAACTTGCATGAAG |
| | TTGGAATACCAAATCCTGATCCAGAGAATAGTTGTTGGTTGGTATT |
| | GGCTACTAGGTCTCTGGATATATGCCATGTCATGGCTGATAGGGA |
| | GATAGAAATGGAGGTTCTCTCTGATGCGGAAGCTTTGGAATTATTT |
| | TACTGTCAACTGGGAGATATAATTGATTCTCCAGAAATTAAGTCCT |
| | ACGCTCAAGCTATTTCCAAGGAATGCTATGGCTCTTCAGTGATAAT |
| | TTCTGCCATTGGAAGTGCCTTAAGAGAGGAAAACAGTATTGAACA |
| | ATGGAAGCTTGCACTGAGAAATCTTCAGTCAGGTGATACAGCTAG |
| | CACAAATGATCATCTTTTTTTTAACCCGCACATAAAATATTGTTAT |
| | GATAGATTGAAAGCTCGTGACGTGAAGAATTTCTTTCTTTACACCG |
| | CATTGTTTCAACAAGATAAAGAAATTGATAGTTCCATGCTGGTAG |
| | ATCGCTTCATTAGTGAAGGCTTAGTTGGTAGGTCGACAGTGGCTGC |
| | TTATAAAAAGGGACATGATATTGTAGTGCGTCTTACAAACGCTTCT |
| | TTGTTAGAGGCTAATGATGACGGTCTAATGGTTAAGATGCACAAT |
| | GTGGTCAGGGATTCAGCATTGGAAATCCTTTCATCAGGTATAGAA |
| | GGTTATCAAGTATTAATGAGATTTCCAGAATCAGATAAGGATGAA |
| | GATCGATCCTTTCACGAAAGTAGCTGGGCTAACAATAACAAGCTG |
| | GTTGACAAGTTCGATTCATTCAGCATCCAGATGCCTAGAATTCACA |
| | AATACTTGACTAGAGCTGGTGCTCATTTGAAGGAATCTCCGTCACC |
| | TGAAGAATGGGAGCAAGCTACTATGATGTTCTTGATGGATAATGA |
| | GTTGTCAAGTCTTCCAAGCAATCCAAGATGCTATAATTTAATAGCA |
| | TTGTTCCTTCAAAGAAACAGCTCCTTAAGGGTGATACCAACATCCT |
| | TTTTTGACAGCATGTCTACTCTTAAAATTCTGAATCTCTCCAAAAC |
| | CAGAATAAAGTCTTTACCTGATTCCTTTTCTAGACTCAAAAGCCTC |
| | CAAGTCTTACTGCTGCGAGATTGTGAGCGCTTGCTTGTGCTTCCTT |
| | CATCAATTGGATTTCATAAATCTCTGAAGGTGCTTGATCTTCAAGG |
| | TACCGAGGTAACCCACTTGCCTGATAGTATTGGTAAATTAGAAAG |
| | CCTGAAACAGTTGAGAGTTTCATTCTATGGATCCATTAATCGTTGT |
| | GAATACAATAAGTTGCCTTCAAAGTTGGTATCAGATGGAATCATTT |
| | CGAGCCTTAAGCTTAGAGAACTTGGTGTTTTTGTTTATCCTGGAGA |
| | TCGACGTTGGACATTGAGTGCATCAGGTATCACGAATGAGATCAG |
| | TAGCCTGAAGTTGTATGTGCTTTATTTTCACTTCCCAAAAGTAGAA |
| | AATCTCGAGCACTTTGTGCATACAAGCCAGTCGTGGAAGGCAAGA |
| | GAGTTGGCTAAATTCAATTTTATTGTTGGACATGATTTCAAACGTA |
| | TTGTATCTTTGGTTTCAAATGATGCAGAACTCTTGTACAACGAAGG |
| | TCAAAGATGTTTAAGATTTGTGAATGGTGAAAGTATACCTGATCC |
| | GGTGGTAGAGGTATTGACACGAGCTACTTCTTTTTACTTAGACCAT |
| | CACCTGAACATCTCCAATCTATCACAATTTGGAATTGACTGCTTTG |
| | ATGGCTTAAAATTTTGCATACTAAGGGATTGTCCGGAGCTGGTAG |
| | GAATTTTGGATAGTCAAGACCAGCGAAGTGCATTCCTTCCTTTTCT |
| | AGAATACTTGAGTATCAACTACCTCTGGAACTTAAAAAGAATTTG |
| | GGTTGGTTATATTGCAGCAGGAAGTTTTGCTAGGTTAAGATGCTTA |
| | TCGATTCATGCTTGTCCAAAATTGAGCTATGTTCTTACTTGCCCCA |
| | TGCTTGATATTCTCCGTAATTTGGAGGAGTTGGTGATTGAAGACTG |
| | TGTATCCCTAAAATATGTTGTGAGTGAAGACGAGGATGTCAAAGA |
| | GATCAAGTATGCTGATGTAGACAGTGTTTCTGCTCTTGAGTTGTCT |
| | CCGCTTCATATGTTGAAAGTATTAAAACTTCATTATCTACCAGAGA |
| | TGTGCGGCATTTGGAGAACCCGGTGGCCTCCATTGGAGTACGTCA |
| | GCTTTTACGATTGTCCCAAGCTCAGAAATCTTCATATGGAAGGGTA |
| | TGATGACACATTCATTAAAGAAATAGCAGCAGATAAAGGGTGGTG |
| | GGATTCTTTAGAGTGGGACGATCTTGAACTGTCAAGAAGACTTCTC |
| | AAGCACGTGACCGAACTCCATGTTGATGAATTGTAAAGAATAGAT |
| | ATATCAACATATATACAGGGGTAACCTCTGCAAACCAATTTTGAA |
| | TTTCTACAGACTGAGTTAAACAAAATTCAGTGGTTATACAACTGGT |
| | ATGATCACTATGTTTATAGTGGGTGTTATGACTCAACATTATTCTC |
| | TGTCCTAAATTTTTACAATACTAGTTTAGTACCTGTGCAACGCACG |
| | ATGATTATTACTCCGTAAATATATTTTTTTTGTAATACTTTTAATAA |
| | TTATTTACATAAAAGTTAATAAAATATATTTTGTAAATGATTTCTT |
| | CATATAACAATTGATTTAATTTTTTACCTAAATAAATAATTTTAAA |
| | AAATTATGTTTAATGATATAAATTGGATACTATCATTGAACTGCAA |
| | TTCAAAATTATACGTTAAAAATGTTTGATTTATTATATTGCATTATT |
| | TTAATTTCCCATAAAATTAATATATATACAAAACTTTCCAAAATTT |
| | TTATTACTGTTGTGAAATACTCCCTCCGTCCCTTAATACTCACACC |
| | ACTTTCCTTTTCGGTTTTACTTTAATACTCGCACCGCTTCTATAAAT |
| | GGAATTTATTTACCAATATTATATTATTTTTCAAACTTACCTACTAA |
| | TCCACTTGTACCCCTACTCCCTATTAAAAAAATCATTTAAGAATTC |
| | ACACCCCCCACTACCCCTTCAGTGGCGGAACTACGTGAGTGACGT |
| | GACCCAGCGAAACTTTGAAAAAAAACCATAGGTATCTAAGATGTT |
| | ATATCATAGAGCCGGTCTCTTATATTATACTAGAAAAACCTAATCC |
| | TGCACATGAGACAACGGTTATAACTACCATATTTTGTGATAAATCT |
| | ATAAGACTTGTTCTTTAAAGTAACATGTCACTTAAAATCGAGTAAC |
| | AGACGGCCTGCTGTATTCAATTCAAATCTAAAGAACAGTTATTTAA |
| | ATACCTGTTCTCTTAAATGGAGTTATTTATTTCTAACATCCACATA |
| | AAAGCTGTAGAAAATAATTGAATAATTTTGTAGGTATTTGATTGTA |
| | ATATAAATATTTATTATTTTTGGGACGATATATATATTTTTTTAATA |
| | TATATTGTGTGTTGTGGACGATGGTGACCCAACACCTAAACATCTT |
| | GGATACGCCACTGTTCCTCTTACACATTCTCCACTAACTATATTAA |
| | AAAAACACCCCACTATCAATTACCACATATTAAATTAATATGTCA |
| | ATTCAAGTGTAAACTCCGTACCGGTCAAATCGGTGCGAGTATTTTT |
| | TAAACCAAATTCGTGATGAATAACATGCATAACTTTTATTTGACAA |
| | CAAAAATCACGTACAAATTAGATTGTATACGGAGTAGTTACTTTTA |
| | ACAAATTAATTTCTTAACTTTTCGACTATGCATTTGTGATGAGATC |
| | TTCCAATAGGTATAAACCATTGGTTAAAATACTGTTTGGAGAAAA |
| | AAATAAAGAGGTATTCCGTCATCATTCTACGACAATAAATATTGA |
| | TTTATTAATGTTGGTTTACATGCATTTTAAAATCCATGCATACATC |
| | GCGTGCATAAAATACTACTCCGTAGTAAAAGATAAAAGGACGGAG |
| | GGACGTAGTAAAAGATAAAAGGACGGAGGGAGTAACATATACAC |
| | CAGGGTATAACACGTGGACAATAAACGGACGGTAACAGGCTAAC |
| | ACAGCTTGTCATCCATAAGGCTAACTTTCCATTTGTAGTCCGCGCT |
| | TGAAAATCAATCAAACTTTTGATGCTTCCCACCAAAAAAACACCC |
| | CAATATTTTTGAACCGAAAATATAAAAATTTCTGCTTGCCTTTCTA |
| | TCACCTTGATCCATCTCTCTCTCCTCAATCCATCTTGTCGCTTCACA |
| | TCTCTCTGATTTGAACTTGAAATCAAAATTGATGATTGGTAAAAAG |
| | CGTCGTACTGTCTCATTGAGTTCTACCCCCGACGATAACTATGTGA |
| | GTCTGCTGATTTATCTCGCAATTTTTTTTTTTATTTACTTTGTATAA |
| | ATGTTCAATTACCCCTTG |
| SEQ ID NO: 6: | GTAAGATGGTGAGTGAAAGTAAGGTGAAAGGTAAAACATGGGAG |
| genomic sequence | GTGATAAATCCAAGGAAATTTTTATTTCGTCCAAGAAAATCCACA |
| of the p10 kinase | CTTTTACAAAGTTGATTTCTTTGGAAGAAGTAAATTTTTACTTGGA |
| gene as present in | TTTATCATTTCCCATAAAACATGAAGGGGTAAATATATGGAGAGA |
| NCIMB 42554, | TAGTTGTGACATTTAATAGGGCGGGTGAGTTTTAATAGGGTGGGG |
| including 2 kb | TTAGCGTTCCACATAACTAATTGCCAACATCTGATTTCCCGAATAC |
| upstream and 2 kb | AAATAAATTTCCATAACAGACCTCAACCCTAGATTATAATTTTGTT |
| downstream | TTTAATTTGTACTCCGTAATTTACAAGATAAAAGAAAATACATTTA |
| | GGATATTAAGAAAAAGAATTAAATGAAATAAAGTAATAAAACAA |
| | TATTACTCCCTCCGTATTTATTTAAGAGATACACTTGGCCGGACAC |
| | GTGAGGTTGAGTAGATATTTTAATAAGAAAAACAAGTGGGGACCA |
| | TGTCATTTGAGGGAGGGGAGAGTGGAAGTGGGGTGTAGATATATT |
| | ATTTAAATAGATGGTGGGGTTGATAAGTTACTAAAAATGGCAAGT |
| | GTATCTCTTAAATAAATACGGCCGGAAAAGGCAAGTGTATCCCTT |
| | AAATAAATACAGAGGGAGTATGATTTTTTGTCAGAAAGGACCTCT |
| | TAAAGTCTGAAAATTATGACAAAGGAATTTATAAAAAAAAATGTT |
| | GTGAGATGAACCTAAAATTGATTTTTTGTTGTGAGATGAAACCTTT |
| | ACCCATTTTTCGATAAAAATTTTCCGGTTTTGACTCGTGTGTGTCG |
| | GTCATGTGCTTCTTAAAAATAATTTCAGATGAACCCCAATTTCCAC |
| | AAAAAGTCAAATTAAACGAATTGATGCGGAAATTGGGCCTTTTTC |
| | TCTTTCCTTGTTTGAATAGGGCGCAACGATTTGTTTTTTTGGTATCT |
| | TGCACAATTGAAGAACCGGTCGAAGAAGATGAACATGAGAGGGA |
| | CCGTAGAGTTAGGAAGGCGCAATTTAGAAGGTGGACCATGGTGCA |
| | CCTTAAGAATATTAGTATATAATTAAAAGAACATCTGGTTATGTAA |
| | AAGGAACATGGACATATATTTTTTTATATTTTTAATAAATTGTGAT |
| | TTTTATAACAAAAAAATAAAATATTATATTGCATGTTCTTTAGTCG |
| | TAGTGTCATGTTCTTTTGTTTATGCACATGTGTTCTTTTGGTGCACA |
| | TGGTGCACCATGGTCCATAGTCCACGTAAAAAAAATCATTTTTTAA |
| | TAAGCACGTGACCAAAGCGCGCGAGTCAACGCCGAAAAATTGGA |
| | GTTACCATCGAAAAATTGATAAAGGTCTCGTCTCACAACAAAAAA |
| | TCAATTTTAGGTCCTATCTCACAACAATTTTTTTTATAAGGTCATTT |
| | ATCACAATTTTTGGACTTTAAAAAAATTTCTAACAAAAAATCATAC |
| | TTCCTCTATTTTTAATAATTGCACCATCTTGGTTTTAACACTATTCC |
| | CATATTCTTATATAGTTATTTTTTTTGTAATTTATATGTAAGGAAAA |
| | ATATATTCATGTGGGATCTTGTTAGATTCGTCTCGAAATATACTTT |
| | CAAATTAGCAAATTTTTATAATTTTTTAAAATGTATAATGGGAGAT |
| | ATTAGTGGTTAAAATAGTGCATTGGCAAGCGTGAAATCAAAGATG |
| | GTACAATTAAAAAAAATGGAGGAAGTATATATTACTATAAATATT |
| | TGTTTCCATCTTTGTATCGACTATTTTAATTACATATTTCCATAGTA |
| | AAAGTTCATGCGACACCATTAAGAATACGCCACATAAAATTGCGA |
| | CAATTAAAACTTATTGCAAGTTGCGACCTTTATCATCACCCTAGTT |
| | GTTTATTTTTTCATGTAAAAAGAATAAAATAGGATAATGTTTGAGT |
| | GGTCTAGTGATGTCTGGGCTTGGTGTTTGTTTCCAATTTCAATCAA |
| | GATCCTCAAATTGGGTACTGCATTGAAGGTAATCATAAAATGCTG |
| | GTGCACGAGTTTGTAGGGGATTGCAGCCTGGATCAGTTGTTGTTTC |
| | CAGACTCCAGCAGTGGGGTAGGAGATCGTTCTGTGCTAGATTGGC |
| | CCATGAGAGTGTCCATATGCTTTAAGATTGTTCGGGTTCTAGAATA |
| | TCTTCACTGCGGGATCAGTCCCCGTGTTGTGCTTCGTGATTTCAAG |
| | CCTGGGAATGTGTTAATGGATTCAAGTTTCAACCCCAAAATAGTCC |
| | CAAGCCGTATTTGTGGAACAATTGGCTACTTGGATATTGAATATTG |
| | TATAACAGGAAGATATTCAGAGAAGACAGATGTGTTTTCTTTTGG |
| | AATTACTCTACTCGAAATCATCAGTGGTAAGAAAAATCTGCGGTTT |
| | ATCTTGGTGAATTGGGCTTGGGACCTACAAGCAAGTGGTAGGATT |
| | CCTGAGATTGTCGATCCTACTCTATCTCAAGTTTCAGAAGATGAAG |
| | TGATACGTTTTGTCAAAGTTGCGCTTTCGTGTGTCCAATATTCAGA |
| | ACTCAGACCGAGCATGTCTGAGGTAAGGGTAATGCTCTCAGACGG |
| | ATATGACTTCAGTAACATGGTACTAACTAGACCATAGAGGAATAA |
| | TTCTTTTGAGTTTTGACATTGATGAATGATATATGATCAACGTTGA |
| | TGTGCCGTAAAAAAGGTAGAAAGAAAATGTACGTTTTAAGTAATA |
| | CCTGCTTCAAACAAACCGACCACTCGCGTTGTATATCTCTACAATA |
| | ATGTTGTAATACTATAACTGTATTAACATATTGCACGCAGTATGAT |
| | TTAAGAACAACAGGACATCATTGATATCAATGATATGAGGAAAAC |
| | CCAACTTTAAGAAACTTTTTACTTCAACTTTCATTGCCTGTGTTGTA |
| | TGGAATGTATTTTTTCATCTGTTTAGAATAATATGTGTTATAATATT |
| | ATTCTAATAATACGGAGTATGTGTTAGAATAATATGAAACAGGGT |
| | AACAATGTCTTATAATATGAAATTTAACATTTATTATCAATAGGGC |
| | TGGTGGCGCAGTTGGCTAGCGCGTAGGTCTCATAGCTAAGATGAG |
| | TGATCCTGAGGTCGAGAGTTCGAGCCTCTCTCACCCCAACTGTAAA |
| | TTTTTTAAAAGAAGCAACTTTTCTTTTTTAACCAGTATGCATTCTAC |
| | ATTCTCTACGCATTTGCTGTTTGCTGTCCATCTGTTCGACAATATGT |
| | CAAAGAACTGATTATATTTGTGTTTTAAACCAAGCTAAGCAGTTCA |
| | AGTTAACATTCTGACATGGCAAAGTAAACTCTACCAGAAATATGA |
| | AAAGAATACAATAAATTTTAGCCAATATTTAATATCAGCACATCTT |
| | GCACATTTCAGTTATTGGTTTACAACATACAGTTATATTAAAAGTT |
| | GGGAACAACTGCAAGTCTGAATGTAAGTAAATACATAGAGTGAAA |
| | ATGGAGTTATGAGGTGCCGGAAATGGACATTAATATCTCGTCTTC |
| | ACATTCTGCAAGAACATGAACAACTGACTCCATGGTTGGCCTTTTA |
| | TTTTTATCATCCTCCACGCAAGAAAGTCCAACCTTAATCATTATAC |
| | CAGCTTGGTATCTACTGAAGTTTCCTTCCAATCTCGGGTCTACAAA |
| | GTCTTCTACCCATGAATCTTCTTCATTCTGCATTTTGCTTTTAGCCA |
| | ATCTCACAATTTTTACCAGTTCTGATATATCCTCTAAGTCATTATG |
| | ATCATCCACCACCCTACTTGACAGTCGAATTCCCTTGACAAGCTCT |
| | AGGATAACAACTCCATAACTGTAGACATCAACCTTTGCAGTAATG |
| | GGTTGGTTCAATGCCCATTCAGGTGCCATATAACCTTTTGTTCCTC |
| | TGATTCCGGTCAGCTCTGCTGAACCCTGACTCCCTCTTTGACGTAG |
| | CTTTGCTAGCCCAAAGTCTGAAATTTTTGGCTCAAAATCCTCATCC |
| | AAGAGTATATTTTCAGGTTTCACATCACAATGGATAACCCATTCTA |
| | GGCACTCATGGTGAAGGTAAGCTAATCCTTTAGCCGTGCCTATTGC |
| | AACTTTAAGCCGCTCTTTCCATCCAACAAGGGTTTGGGTGGAGAA |
| | CAAGTGTCTGTCCAAAGATCCATTTTCCACAAGCTCATAAACCAA |
| | GAGCTTGTGTTTCCTTTCGGAACAAAAACCCCACATTCTTGCAAGG |
| | TTCATATGATTGATTTTACCAATTGTACTCACTTCTGCCCAAAATTC |
| | TTCTTCTCCTTGGAATATATTTTCTAGTTTCTTCACTGCAACTGCTC |
| | TCTCATCTGCCAGAATACCCTTGTAAACAGCACCAAACCCTCCCTT |
| | TCCTAGAACTTCCTTAAATTTCCCTGTTGCAGTCTTGAGCTCGTTGT |
| | AGCTAAAGCTCCTAAACTGATTTGAAATGGCACGATATCCATCTTC |
| | CATAGAAGTTGAAAGACCATGCTTCTTATAAAGAAACCACCAAGC |
| | TGCTACCAAAAGGACTAGTTCAAGAATACCAATGACAGAAGCAAA |
| | TGCGTAGATATAAACCCACTTGAATCTTTGAATGGTGGTGTCATAT |
| | GTGATAGGCAATTCAACTACTTTACTTTGCTCGTTCCCACATTCAA |
| | GACGAGACACATCAAAGC |
| SEQ ID NO: 7: | ATGACTGGTACTTGTTTCGGTGATTTGGGTGGTGTGAGGAGCTGTT |
| coding sequence of | GGGGTTATAAAGTGAAATTGGTGCGTAATCAAGAGGAAATTAAAG |
| wildtype p10 NLR- | ACGATACCCTATTAAAAAATGTCAGATTGTTTACCTGGAAAGAGC |
| kinase gene not | TGCAGAAGGCAACAGATAACTTTAACTCCAGCTCACTTATAGGCA |
| conferring | AGGGTGGGTTTACATCTTACTATAAGGGCACTTTAGAAGATGGGA |
| resistance, as | CTGAGGTGGCAGTGCTATCACATAGAAAGGGGGGTGTAAATCAGA |
| present in Sp75 | AGCGAGATTCGGATTTTACTTCTTTTATTAATGTGATGTCCAGAAT |
| | CCGGCATCGGAATGTGGTTGAGTTGATCGGTTGCTGTATTGAGGGT |
| | GATCATATGATTTTTGTGTATGAGTTTCTTGGGTATGACAGGCTGG |
| | ATCAGTTGTTAGTTAGTAAGTCTGAGGCGATAGATTGGCCTACTAG |
| | AGCTTCTATATGCATTGGCATTGCTCGTGGTCTTGACTATCTTCAA |
| | GAGGGGGCTAGTCGTCGTCTTGTACATCGTGCTATCAATCCTGATA |
| | ATATATTTCTTGATAGTAACTTCAATCCCAAAATAGCAGGTTTCCG |
| | CATAGCACAGTTGTTTGATGATGATGAATCACAATATAGTGGAGT |
| | ATATACGGCATGCTTACCATCTTCATATTATACGGATCCTCAATAC |
| | TTGGCTACAGGAAGATGTACAGAGAAGGTTGATGTTTATAGCTTT |
| | GGAATTATTTTACTTGAAGTCATCAGTGGCAGGAGAGTTCACGAG |
| | TTTCAGAGAGATGGCAGGAGAGTTCACAAGTTTCAGAGAGATGGC |
| | AGTGGGGTTACGCTGGTGGATTGGATTTTGGAACTACGGAAAACT |
| | GATATGATTCGTGAGATTGTTGATCCAGCTTTATCTGACTTTCCAG |
| | AGGATGAGATGATACGCTTTATTGATGTTGCACTCTCTTGTGTTCA |
| | GTACTCATCAAAGCTCAGACCAAACACGTCGGAGGTTTTGCTTAT |
| | GCTCTTGGGAGGTTATGACTTCAGTAAGAAGGTATTAAGAAATCT |
| | GGAACAATCTTGGCAGTCTGGCTATCATGATGAAGTTGGCAGTCTT |
| | AGTCATGATAAAATTTCTTCAACTCCTGTACCTAGAAGGAAACAA |
| | GATTTTCTAGTGGCAGACACCACGACAAGAAGGATTCCAAGTCAG |
| | CCAGCTACTGAATCAATCAGTAGGACGACACGTGGTTGCGAAATA |
| | GAAATAGAGAGTGACCTTGACACACATTCTGGTGCATCAGAACAA |
| | ATGGAGATTTCACATGATCTAGAAACTGAATTATACAGATCTATTG |
| | AGGCAAACATCTCTGCTGCAGCTTCTGTACCTGGACCAGGTGACA |
| | AAAATGCATTGAAACCTGAAGTTGTCAGCCCATCTTCTCCAAGTAC |
| | TAACAATGGAAGCCCTTCAACTGTAAGTACTGATATTCATGTCCCT |
| | GGAGAAGCGTTCGAGACAACAGAGCATTCGCAGATGTCACATGAT |
| | CTAGAAGCTGAATTTCAAATGAGCGAACCATCTTTATCAGAGTTTC |
| | TGTTTGGCCAAATTGCTATGAAAAGTGATGTAAGGGCTACTTCATC |
| | TGTGGAAATTAACAATTGCGTCCAGAATGAAGCAGTCGACAGGTT |
| | TTCATTTCAGGCTGAAATCAGTGGAACAGAGGAGGTTCTTTCACTA |
| | CCAAAAGTTAATATGAAGAGATCAATTCATTGTACTCTAGAGAAG |
| | ATTTTTGAAGCCTTTCAGGATTCTAATCGCAGAAGAATAGGTGTTT |
| | ATGGAAGAGGGGGTAGTGGCAAAACTACCATGCTAAAAAACTTGT |
| | TCCATCATTCTGCAACAAAGGAGTTGTTTGACCAAGTCCTTTTTGT |
| | GACTGTCCCCAGGTTTGCTCATCAAAAACATGTACGAGATGAGAT |
| | TGTACAACAGTTGTCATTGAATGTACAAGAGTCCTGCTCAGAGCA |
| | TGAAATTTCACATCTTATTCATCAGGCTCTAGTAGACGGAAAGTTT |
| | CTGGTGCTTTTGGATGATGTTTGGGAATCAATTAACTTGCATGAAG |
| | TTGGAATACCAAATCCTGATCCAGAGAATAGTTGTTGGTTGGTATT |
| | GGCTACTAGGTCTCTGGATATATGCCATGTCATGGCTGATAGGGA |
| | GATAGAAATGGAGGTTCTCTCTGATGCGGAAGCTTTGGAATTATTT |
| | TACTGTCAACTGGGAGATATAATTGATTCTCCAGAAATTAAGTCCT |
| | ACGCTCAAGCTATTTCCAAGGAATGCTATGGCTCTTCAGTGATAAT |
| | TTCTGCCATTGGAAGTGCCTTAAGAGAGGAAAACAGTATTGAACA |
| | ATGGAAGCTTGCACTGAGAAATCTTCAGTCAGGTGATACAGCTAG |
| | CACAAATGATCATCTTTTTTTTAACCCGCACATAAAATATTGTTAT |
| | GATAGATTGAAAGCTCGTGACGTGAAGAATTTCTTTCTTTACACCG |
| | CATTGTTTCAACAAGATAAAGAAATTGATAGTTCCATGCTGGTAG |
| | ATCGCTTCATTAGTGAAGGCTTAGTTGGTAGGTCGACAGTGGCTGC |
| | TTATAAAAAGGGACATGATATTGTAGTGCGTCTTACAAACGCTTCT |
| | TTGTTAGAGGCTAATGATGACGGTCTAATGGTTAAGATGCACAAT |
| | GTGGTCAGGGATTCAGCATTGGAAATCCTTTCATCAGGTATAGAA |
| | GGTTATCAAGTATTAATGAGATTTCCAGAATCAGATAAGGATGAA |
| | GATCGATCCTTTCACGAAAGTAGCTGGGCTAACAATAACAAGCTG |
| | GTTGACAAGTTCGATTCATTCAGCATCCAGATGCCTAGAATTCACA |
| | AATACTTGACTAGAGCTGGTGCTCATTTGAAGGAATCTCCGTCACC |
| | TGAAGAATGGGAGCAAGCTACTATGATGTTCTTGATGGATAATGA |
| | GTTGTCAAGTCTTCCAAGCAATCCAAGATGCTATAATTTAATAGCA |
| | TTGTTCCTTCAAAGAAACAGCTCCTTAAGGGTGATACCAACATCCT |
| | TTTTTGACAGCATGTCTACTCTTAAAATTCTGAATCTCTCCAAAAC |
| | CAGAATAAAGTCTTTACCTGATTCCTTTTCTAGACTCAAAAGCCTC |
| | CAAGTCTTACTGCTGCGAGATTGTGAGCGCTTGCTTGTGCTTCCTT |
| | CATCAATTGGATTTCATAAATCTCTGAAGGTGCTTGATCTTCAAGG |
| | TACCGAGGTAACCCACTTGCCTGATAGTATTGGTAAATTAGAAAG |
| | CCTGAAACAGTTGAGAGTTTCATTCTATGGATCCATTAATCGTTGT |
| | GAATACAATAAGTTGCCTTCAAAGTTGGTATCAGATGGAATCATTT |
| | CGAGCCTTAAGCTTAGAGAACTTGGTGTTTTTGTTTATCCTGGAGA |
| | TCGACGTTGGACATTGAGTGCATCAGGTATCACGAATGAGATCAG |
| | TAGCCTGAAGTTGTATGTGCTTTATTTTCACTTCCCAAAAGTAGAA |
| | AATCTCGAGCACTTTGTGCATACAAGCCAGTCGTGGAAGGCAAGA |
| | GAGTTGGCTAAATTCAATTTTATTGTTGGACATGATTTCAAACGTA |
| | TTGTATCTTTGGTTTCAAATGATGCAGAACTCTTGTACAACGAAGG |
| | TCAAAGATGTTTAAGATTTGTGAATGGTGAAAGTATACCTGATCC |
| | GGTGGTAGAGGTATTGACACGAGCTACTTCTTTTTACTTAGACCAT |
| | CACCTGAACATCTCCAATCTATCACAATTTGGAATTGACTGCTTTG |
| | ATGGCTTAAAATTTTGCATACTAAGGGATTGTCCGGAGCTGGTAG |
| | GAATTTTGGATAGTCAAGACCAGCGAAGTGCATTCCTTCCTTTTCT |
| | AGAATACTTGAGTATCAACTACCTCTGGAACTTAAAAAGAATTTG |
| | GGTTGGTTATATTGCAGCAGGAAGTTTTGCTAGGTTAAGATGCTTA |
| | TCGATTCATGCTTGTCCAAAATTGAGCTATGTTCTTACTTGCCCCA |
| | TGCTTGATATTCTCCGTAATTTGGAGGAGTTGGTGATTGAAGACTG |
| | TGTATCCCTAAAATATGTTGTGAGTGAAGACGAGGATGTCAAAGA |
| | GATCAAGTATGCTGATGTAGACAGTGTTTCTGCTCTTGAGTTGTCT |
| | CCGCTTCATATGTTGAAAGTATTAAAACTTCATTATCTACCAGAGA |
| | TGTGCGGCATTTGGAGAACCCGGTGGCCTCCATTGGAGTACGTCA |
| | GCTTTTACGATTGTCCCAAGCTCAGAAATCTTCATATGGAAGGGTA |
| | TGATGACACATTCATTAAAGAAATAGCAGCAGATAAAGGGTGGTG |
| | GGATTCTTTAGAGTGGGACGATCTTGAACTGTCAAGAAGACTTCTC |
| | AAGCACGTGACCGAACTCCATGTTGATGAATTGTAA |
| SEQ ID NO: 8: | ATGGGGGGTTGTTTGTCATATAGATTGAAGTTGATCCGCGATCTAA |
| coding sequence of | ACCCAAATGAGGATACTTGGTTAGGAAACGTGACTAGTTTTTCCTA |
| wildtype p10 | CAAATCACTGAAAAAGGCAACAAACAATTTCAGCAATAGGCTCAG |
| kinase gene not | ATCGAATGGATTTTCATCATCTTACAAGGGCACTTTAACAGATGGG |
| conferring | TTACAAGTCATAGTTAACGTATATAGAACCGATTATAGTGTCATAG |
| resistance, as | ACTTGGCTTCAATAATCAAAATCATGTCTGGAGTCAGGCACCAAA |
| present in Sp75 | ATGTAGTGGAACTCATTGGGTACTGCATTGAAGGTAATCATAAAA |
| | TGCTGGTGCACGAGTTTGTAGGGGATTGCAGCCTGGATCAGTTGTT |
| | GTTTCCAGACTCCAGCAGTGGGGTAGGAGATCGTTCTGTGCTAGA |
| | TTGGCCCATGAGAGTGTCCATATGCTTTAAGATTGTTCGGGTTCTA |
| | GAATATCTTCACTGCGGGATCAGTCCCCGTGTTGTGCTTCGTGATT |
| | TCAAGCCTGGGAATGTGTTAATGGATTCAAGTTTCAACCCCAAAA |
| | TAGTAGGTTTTGAGTGTTCAAGTCTTCTAGAAGACAGTGAATTAGA |
| | CCGTAGTAGCCGTATTTGTGGAACAATTGGCTACTTGGATATTGAA |
| | TATTGTATAACAGGAAGATATTCAGAGAAGACAGATGTGTTTTCTT |
| | TTGGAATTACTCTACTCGAAATCATCAGTGGTAAGAAAAATCTGC |
| | GGTTTATCTTGGTGAATTGGGCTTGGGACCTACAAGCAAGTGGTA |
| | GGATTCCTGAGATTGTCGATCCTACTCTATCTCAAGTTTCAGAAGA |
| | TGAAGTGATACGTTTTGTCAAAGTTGCGCTTTCGTGTGTCCAATAT |
| | TCAGAACTCAGACCGAGCATGTCTGAGGTAAGGGTAATGCTCTCA |
| | GACGGATATGACTTCAGTAACATGGTACTAACTAGACCATAG |
| SEQ ID NO: 9: | MTGTCFGDLGGVRSCWGYKVKLVRNQEEIKDDTLLKNVRLFTWKEL |
| amino acid | QKATDNFNSSSLIGKGGFTSYYKGTLEDGTEVAVLSHRKGGVNQKR |
| sequence of the | DSDFTSFINVMSRIRHRNVVELIGCCIEGDHMIFVYEFLGYDRLDQLL |
| wildtype p10 NLR- | VSKSEAIDWPTRASICIGIARGLDYLQEGASRRLVHRAINPDNIFLDSN |
| kinase not | FNPKIAGFRIAQLFDDDESQYSGVYTACLPSSYYTDPQYLATGRCTEK |
| conferring | VDVYSFGIILLEVISGRRVHEFQRDGRRVHKFQRDGSGVTLVDWILEL |
| resistance, as | RKTDMIREIVDPALSDFPEDEMIRFIDVALSCVQYSSKLRPNTSEVLLM |
| present in Sp75 | LLGGYDFSKKVLRNLEQSWQSGYHDEVGSLSHDKISSTPVPRRKQDF |
| | LVADTTTRRIPSQPATESISRTTRGCEIEIESDLDTHSGASEQMEISHDL |
| | ETELYRSIEANISAAASVPGPGDKNALKPEVVSPSSPSTNNGSPSTVST |
| | DIHVPGEAFETTEHSQMSHDLEAEFQMSEPSLSEFLFGQIAMKSDVRA |
| | TSSVEINNCVQNEAVDRFSFQAEISGTEEVLSLPKVNMKRSIHCTLEKI |
| | FEAFQDSNRRRIGVYGRGGSGKTTMLKNLFHHSATKELFDQVLFVTV |
| | PRFAHQKHVRDEIVQQLSLNVQESCSEHEISHLIHQALVDGKFLVLLD |
| | DVWESINLHEVGIPNPDPENSCWLVLATRSLDICHVMADREIEMEVLS |
| | DAEALELFYCQLGDIIDSPEIKSYAQAISKECYGSSVIISAIGSALREENS |
| | IEQWKLALRNLQSGDTASTNDHLFFNPHIKYCYDRLKARDVKNFFLY |
| | TALFQQDKEIDSSMLVDRFISEGLVGRSTVAAYKKGHDIVVRLTNASL |
| | LEANDDGLMVKMHNVVRDSALEILSSGIEGYQVLMRFPESDKDEDRS |
| | FHESSWANNNKLVDKFDSFSIQMPRIHKYLTRAGAHLKESPSPEEWE |
| | QATMMFLMDNELSSLPSNPRCYNLIALFLQRNSSLRVIPTSFFDSMSTL |
| | KILNLSKTRIKSLPDSFSRLKSLQVLLLRDCERLLVLPSSIGFHKSLKVL |
| | DLQGTEVTHLPDSIGKLESLKQLRVSFYGSINRCEYNKLPSKLVSDGII |
| | SSLKLRELGVFVYPGDRRWTLSASGITNEISSLKLYVLYFHFPKVENL |
| | EHFVHTSQSWKARELAKFNFIVGHDFKRIVSLVSNDAELLYNEGQRC |
| | LRFVNGESIPDPVVEVLTRATSFYLDHHLNISNLSQFGIDCFDGLKFCI |
| | LRDCPELVGILDSQDQRSAFLPFLEYLSINYLWNLKRIWVGYIAAGSF |
| | ARLRCLSIHACPKLSYVLTCPMLDILRNLEELVIEDCVSLKYVVSEDE |
| | DVKEIKYADVDSVSALELSPLHMLKVLKLHYLPEMCGIWRTRWPPLE |
| | YVSFYDCPKLRNLHMEGYDDTFIKEIAADKGWWDSLEWDDLELSRR |
| | LLKHVTELHVDEL |
| SEQ ID NO: 10: | MGGCLSYRLKLIRDLNPNEDTWLGNVTSFSYKSLKKATNNFSNRLRS |
| amino acid | NGFSSSYKGTLTDGLQVIVNVYRTDYSVIDLASIIKIMSGVRHQNVVE |
| sequence of the | LIGYCIEGNHKMLVHEFVGDCSLDQLLFPDSSSGVGDRSVLDWPMRV |
| wildtype p10 | SICFKIVRVLEYLHCGISPRVVLRDFKPGNVLMDSSFNPKIVGFECSSL |
| kinase not | LEDSELDRSSRICGTIGYLDIEYCITGRYSEKTDVFSFGITLLEIISGKKN |
| conferring | LRFILVNWAWDLQASGRIPEIVDPTLSQVSEDEVIRFVKVALSCVQYS |
| resistance, as | ELRPSMSEVRVMLSDGYDFSNMVLTRP |
| present in Sp75 | |
| SEQ ID NO: 11: | TACTGAAAGTAATAATTCTGCAATTGAATTGTGGTTAGAAAA |
| genomic sequence | GATTAAGTAATGCAAGATAGTGTACCTGGAAGGCTGGAAGA |
| of the wildtype p10 | AACTACGGAAGTTAGAAATTGAAATTCAGAACTGAGGAATG |
| NLR-kinase gene | AAGAATGAAGAATGAAGAATGAAGAATTGACTTAACCCAAA |
| not conferring | AACTGGACTTGCATTCGAGCTAGATTTTAAAATTTGACTTAA |
| resistance, as | CCCATTATCTGAGTCTGGTAAGTTGCGTTGGATTCACATTAT |
| present in Sp75, | TTTCATTTATTTTTTCTACGAATCTTTTAAAGTTGTCACTTTT |
| including 2 kb | GAAATAACTACTCCCTCCGTCCCATAATTATCTTCTTGTTTGA |
| upstream and 2 kb | CCAAAAACACGGATTTTAAGAAAAGTGTAATATAGTACATG |
| downstream | AAAAAGTGGAATAAAGTACAAATGATGATTGAGTTATATGA |
| | AAAAGTGGAATAAAGTACATGTGAAAGAGAATATACTACAT |
| | GGGAAAAGTAGAATATAGTACATGGGTGGGGTTTTCAATTC |
| | AATTTTAATTAATATAGTATATGAGAAAGTGGGAACCTTAGT |
| | AGCCAAAATTAGAAACAGGAAGATAATTTTGGGACGCTCAT |
| | TTAGGAAAGCAGGAAGATAATTTTGGGACGGAGGGAGTAGA |
| | ACCTTATAGGTTTTTTTTAATATATCCAACTCAAACTTTCTCC |
| | GTTAAGAAAATCACCGTCCAATTCTAATTCCATCAAAATTAT |
| | TGTTAGTGGGGTCCACATCCAACGACCAAATTATGTTTTTCC |
| | TCCATTATATCGTAAAACATAAATGATATAATGAGAGGGAG |
| | AGGTGAGGAGAGGTTTCTTGTCCGCTTTCGTTGTGTTTTCATT |
| | GTGAAATAATATTAAAACATTTTAAATATACATGTGATTTTA |
| | CATAGAGTATAATGGTTTTACCCTCGTGCTTCTTAAGAGTAG |
| | GAAGAGCTTGGCCTTGATTCAAATACTATAATGAAGCTATTA |
| | CAGTTGATATATTTATTCATACTTGGGTGACAGATAAAAATG |
| | CTTAATCTATTCCTAAATCTAATAATATGATGAGTTTTCCGA |
| | TCATCAAATAAACAATTGCAATGTATACGTAGTACTCCGTGA |
| | TACTCCCTCCATCCCTTAATAATCGCACCGCTTTTCTTTTCGG |
| | GCCGTCCCTTAATACTTGCACCGCTTCTATAAATAGAATTAT |
| | TCACCAATGTTATATTATTTCGCACACTTACCTACTAACCCA |
| | TTCACACTCCCACCCCCCACTTCTCACCCCTTACCCATTTTTC |
| | ACTAATTATATTAAAAAAATACGGAGTACTCCACTATCAAAT |
| | AACACCTATTAAATTAATAAGTGAATTCAAGTGACTTAAATT |
| | CCACACCGGTCAAACCAATACGAGTATTAAAGGACGAAGGG |
| | AGTAGTTATTTTTACACTTCAACCAAAAAAAACTTGGAAAAC |
| | CTGGCTACACTAATTTTGTAAAATCTGAAAAGTCAAAATAA |
| | AAATCACTCACGGTCCACATAGACTGCTGCATCCGTTTTCCC |
| | CCACACAATGCTCCGACATCGGATGATTTCCGGGAAAAAAA |
| | ATCTAATTGTTTTAAGTCAGACTGATTACACAAAAATTGACT |
| | TTTGACGAGCAATTCTCCAGTCAGTGTGTTACGGTGTCATTG |
| | ATGCGGAATCTCCGGTGCTTTCTTTTATTAATCAAATCTCTAC |
| | ATTTCAATCCAATTCCAGGTACATAATCTACTGTTCTTTTCAC |
| | GCAAATTAGGTCCTACACTCGAGAAGAACATACAATTCATC |
| | CTCCATGTTGCATATATAATGCTCTCGTGTTTACAATCTAAC |
| | AATTGCTAAATTCATTGCATGAACTTATTTTGTAATTTAATC |
| | AGAATGACTTGTAATTCTTCATTTGTTGTTGTAATTCCTTTTC |
| | AGATGAGTTAATTTCCGTTGAAATTATGTGAATTTTGGACAG |
| | CCCATCGTCTTTAAACTGCTGAATTTGTCTGAATTTGATCTG |
| | GGTATTGGAGAAATTGCAGTGATAATCAGAAATGACTGGTA |
| | CTTGTTTCGGTGATTTGGGTGGTGTGAGGAGCTGTTGGGGTT |
| | ATAAAGTGAAATTGGTGCGTAATCAAGAGGAAATTAAAGAC |
| | GATACCCTATTAAAAAATGTCAGATTGTTTACCTGGAAAGA |
| | GCTGCAGAAGGCAACAGATAACTTTAACTCCAGCTCACTTAT |
| | AGGCAAGGGTGGGTTTACATCTTACTATAAGGGCACTTTAG |
| | AAGATGGGACTGAGGTGGCAGTGCTATCACATAGAAAGGGG |
| | GGTGTAAATCAGAAGCGAGATTCGGATTTTACTTCTTTTATT |
| | AATGTGATGTCCAGAATCCGGCATCGGAATGTGGTTGAGTT |
| | GATCGGTTGCTGTATTGAGGGTGATCATATGATTTTTGTGTA |
| | TGAGTTTCTTGGGTATGACAGGCTGGATCAGTTGTTAGTTAG |
| | TAAGTCTGAGGCGATAGATTGGCCTACTAGAGCTTCTATATG |
| | CATTGGCATTGCTCGTGGTCTTGACTATCTTCAAGAGGGGGC |
| | TAGTCGTCGTCTTGTACATCGTGCTATCAATCCTGATAATAT |
| | ATTTCTTGATAGTAACTTCAATCCCAAAATAGCAGGTTTCCG |
| | CATAGCACAGTTGTTTGATGATGATGAATCACAATATAGTGG |
| | AGTATATACGGCATGCTTACCATCTTCATATTATACGGATCC |
| | TCAATACTTGGCTACAGGAAGATGTACAGAGAAGGTTGATG |
| | TTTATAGCTTTGGAATTATTTTACTTGAAGTCATCAGTGGCA |
| | GGAGAGTTCACGAGTTTCAGAGAGATGGCAGGAGAGTTCAC |
| | AAGTTTCAGAGAGATGGCAGTGGGGTTACGCTGGTGGATTG |
| | GATTTTGGAACTACGGAAAACTGATATGATTCGTGAGATTGT |
| | TGATCCAGCTTTATCTGACTTTCCAGAGGATGAGATGATACG |
| | CTTTATTGATGTTGCACTCTCTTGTGTTCAGTACTCATCAAAG |
| | CTCAGACCAAACACGTCGGAGGTTTTGCTTATGCTCTTGGGA |
| | GGTTATGACTTCAGTAAGAAGGTATTAAGAAATCTGGAACA |
| | ATCTTGGCAGTCTGGCTATCATGATGAAGTTGGCAGTCTTAG |
| | TCATGGTGAGTTATTTATAAAAAAAAAAAAAATCGTTTCGCT |
| | TCATGAAATTTAGATCAATATAAGCTTGCCTTTACCGTAAGT |
| | CATGGTTTGATTTCAAACAATGCCATTTTCTGCTTTTGCTTTT |
| | TGTATATGATAACAGCTTGCTAGGATGACTTCTTGATTGGCT |
| | TTAGGAACCATGTCTTCTTTCACGTCTGCTTGATGATCTTTGA |
| | ATGTTATTGCAATCAAGTTTATTGTGTAATTTGATGAAGTGA |
| | AGTAGTAAAACATACATAGTAGTAATTAGTAAAAACACCTA |
| | GCCCATGTTGTTAGATGGGTAGGACATAAATCATCATAAGT |
| | GCTTCATTTATAATTTGGATTTGTATGAACAAAAGTTATTTCT |
| | GAAATAATTTCAAATCTTCAATCATCTCATACATATTATTTG |
| | TAGTGAACAATTTTAGAAGTTTCTGAAAAAGTCCTCATTTTT |
| | TGTAAGTCGTGATGATGGAGATTAGTATTCAGGGACTTTGAC |
| | CATTGAACCTATTGATCTCAATGTAGGGTAGATGTCATCAAA |
| | GACTGGGTATCATAATTTATGAGATGGAATTTTTTTTATTCA |
| | AAGACTTCTTCCAACTTTTTTTGGTGTCCTTTTCCGCCATTTG |
| | GATGGGATGTGGTGTGGGGTGGGGTGGGGGGGTTATTTTGC |
| | TTGAGCTAAGTGGCTAGTTTTGTAGAAGGCATGTTGACCATA |
| | ACTAAGGTCAACTTTTTGTTGTAGTTGGGAAGTTTCTGGAGT |
| | ATGCTTTTTCAGTATCCAGTAAATAATCCAATCTATCTGCTTT |
| | TTGGGCATACTCCTGTTGTCCAAGGGAAATGGGGACTTATTT |
| | TAAATAAGAATAATTGTTATATCGTCATGTTCATTAGTGAGC |
| | AGATAATCTGCAGCAATTGTTCGTTTTGAGGAATGTCCTTTT |
| | TTTGTCTTCCCTTCTTATTTTATTCTTGGTGGACATATCATCTT |
| | GACTGTTCACACAAAAAACACAAACTACATACTCTAAGTCT |
| | ACAAATAACATGAACCAAAAGGTGTAACTCGTATACAACCC |
| | ATAAATACACTACCAAAATTGCTTTCAGAAACGATATGGAA |
| | AAATGGAAAACTGTGAACATGATATGAACCATAGATTGCTT |
| | GGGTTTAACTAGTTTCGCTTTCAACAAAATGAGAGGACTAGT |
| | TTTGTTACTTTTTTAAAGCACTTTGGACAAGTAATCCCTCTTA |
| | TATTTGAGTTTATTGATCGTTTGATTTCTGAATGCAGTATGAT |
| | AAGCTGCACAGTTTTCTAAGTGCTTGTTCTTTTAAATGGAGT |
| | AAATTTTTGAAATATCATTATGTGTTCTCCTTAACACCACCA |
| | AAACAAAATTACATGCTAAAGAAAGTATAAGTGGAAATTGA |
| | CAACATATATTGGCTATCGGAAACTAATTGCACTTGAAATCA |
| | TTCACTAGGATCTTCTTTCAGTGATCATTTTTAAGGAATATG |
| | GTCTAAACTCTGAGTCTTTACTTTATCCCTATGTCGATATAA |
| | AGATAAAAATACAACGGATTATAACTCAAACTACTATACAA |
| | GTATACATGTATAAGTAGCTACATATAATGTGACACTTCATC |
| | AATGACTACTGTGGTATGTGCAAACAATGAATGACACAGTA |
| | CTCATCAGACATTTAAATTCCATATTCTCCTATTTCAAGTACT |
| | CCCTCCGTCCCATAATTATAGTCCCGTTTGACCAAAAACACG |
| | AATTTTAAGAAAAATGGAATATAGTACATAAAAAAGTATAA |
| | TAACGTACAAATGATGATTGGTTTGTATGGAAAAGTGTAAT |
| | AAAGTATGAGAAAGAAAAATATAGTACAAGGGAAAGTGGA |
| | ATATAGTACTTATTTTGTTTTGTTGTGTTTTCAATGCATTTTTT |
| | AATTAATATGGTACATGAGAAAGTGGGGACCTTAATAGACA |
| | AAAATAGAAACAGGATTATAATTTTGGGACGCCCGATTTAG |
| | AAAACAGGACTATAATTTTGGGACGGAGGGAGTAATCCATA |
| | AGGAAATGCATGCATCAATTACAACTTACAACTCTTACCTTC |
| | AGCCAATTAGAAATCAGCTTTCCAGATCTAATTAGCAAAAA |
| | ACAAGTGGGATTGTGTCATAGCTTAACTGAAAAATCAGATTT |
| | GTTGCTTAATAGCTTAACTGAAAAAAATCAGATTTTTACTCT |
| | GGTGTCTGTTCAGGTGGTCCTTTGTCTTTTGATTTGATGTGGG |
| | ACAATGTCACCTTTTTTGGCGGCATTATGGGCTAATGCGGCG |
| | GGGACTTTTGGGAGGTTCGAAGGAAATGGCTTAATCTATAA |
| | CTTGCCTTTACAGAAGAACTTGTTCTCTTATGTTTATACTCCT |
| | TTTTCAAAGGTGTTACATAATACTTTCTTTTTTTATATATATA |
| | AAATGAACATTTATATGAAGGAATGGTAGTAAGTCTAGTAA |
| | CCTGCCTATGCTGCTATGCATGATGCAACTGTTGCTAATTAA |
| | ATCGTACCAAAATCTGTTATTATCAAGTTTTGAGTATATAAA |
| | CATTCATATAGCTCGTGGTTAAAAGCCATTTTGTCCCAAAGG |
| | GACTGAGTCTCTGGATATGGCTTTGGTTTGACGACCACAATA |
| | GTGTTGTTATGCTGAAATTGCTGTTTGGTATTGAGGCTCATTT |
| | ATATCTCTGGATTTTGGGACATACAGTTAATTTCTTATTCTGT |
| | TTTCCTAATATGGCTACTTTTACAGATAAAATTTCTTCAACTC |
| | CTGTACCTAGAAGGAAACAAGATTTTCTAGTGGCAGACACC |
| | ACGACAAGAAGGATTCCAAGTCAGCCAGCTACTGAATCAAT |
| | CAGTAGGACGACACGTGGTTGCGAAATAGAAATAGAGAGTG |
| | ACCTTGACACACATTCTGGTGCATCAGAACAAATGGAGATTT |
| | CACATGATCTAGAAACTGAATTATACAGATCTATTGAGGCA |
| | AACATCTCTGCTGCAGCTTCTGTACCTGGACCAGGTGACAAA |
| | AATGCATTGAAACCTGAAGTTGTCAGCCCATCTTCTCCAAGT |
| | ACTAACAATGGAAGCCCTTCAACTGTAAGTACTGATATTCAT |
| | GTCCCTGGAGAAGCGTTCGAGACAACAGAGCATTCGCAGAT |
| | GTCACATGATCTAGAAGCTGAATTTCAAATGAGCGAACCAT |
| | CTTTATCAGAGTTTCTGTTTGGCCAAATTGCTATGAAAAGTG |
| | ATGTAAGGGCTACTTCATCTGTGGAAATTAACAATTGCGTCC |
| | AGAATGAAGCAGTCGACAGGTTTTCATTTCAGGCTGAAATC |
| | AGTGGAACAGAGGAGGTTCTTTCACTACCAAAAGTTAATAT |
| | GAAGAGATCAATTCATTGTACTCTAGAGAAGATTTTTGAAGC |
| | CTTTCAGGATTCTAATCGCAGAAGAATAGGTGTTTATGGAAG |
| | AGGGGGTAGTGGCAAAACTACCATGCTAAAAAACTTGTTCC |
| | ATCATTCTGCAACAAAGGAGTTGTTTGACCAAGTCCTTTTTG |
| | TGACTGTCCCCAGGTTTGCTCATCAAAAACATGTACGAGATG |
| | AGATTGTACAACAGTTGTCATTGAATGTACAAGAGTCCTGCT |
| | CAGAGCATGAAATTTCACATCTTATTCATCAGGCTCTAGTAG |
| | ACGGAAAGTTTCTGGTGCTTTTGGATGATGTTTGGGAATCAA |
| | TTAACTTGCATGAAGTTGGAATACCAAATCCTGATCCAGAG |
| | AATAGTTGTTGGTTGGTATTGGCTACTAGGTCTCTGGATATA |
| | TGCCATGTCATGGCTGATAGGGAGATAGAAATGGAGGTTCT |
| | CTCTGATGCGGAAGCTTTGGAATTATTTTACTGTCAACTGGG |
| | AGATATAATTGATTCTCCAGAAATTAAGTCCTACGCTCAAGC |
| | TATTTCCAAGGAATGCTATGGCTCTTCAGTGATAATTTCTGC |
| | CATTGGAAGTGCCTTAAGAGAGGAAAACAGTATTGAACAAT |
| | GGAAGCTTGCACTGAGAAATCTTCAGTCAGGTGATACAGCT |
| | AGCACAAATGATCATCTTTTTTTTAACCCGCACATAAAATAT |
| | TGTTATGATAGATTGAAAGCTCGTGACGTGAAGAATTTCTTT |
| | CTTTACACCGCATTGTTTCAACAAGATAAAGAAATTGATAGT |
| | TCCATGCTGGTAGATCGCTTCATTAGTGAAGGCTTAGTTGGT |
| | AGGTCGACAGTGGCTGCTTATAAAAAGGGACATGATATTGT |
| | AGTGCGTCTTACAAACGCTTCTTTGTTAGAGGCTAATGATGA |
| | CGGTCTAATGGTTAAGATGCACAATGTGGTCAGGGATTCAG |
| | CATTGGAAATCCTTTCATCAGGTATAGAAGGTTATCAAGTAT |
| | TAATGAGATTTCCAGAATCAGATAAGGATGAAGATCGATCC |
| | TTTCACGAAAGTAGCTGGGCTAACAATAACAAGCTGGTTGA |
| | CAAGTTCGATTCATTCAGCATCCAGATGCCTAGAATTCACAA |
| | ATACTTGACTAGAGCTGGTGCTCATTTGAAGGAATCTCCGTC |
| | ACCTGAAGAATGGGAGCAAGCTACTATGATGTTCTTGATGG |
| | ATAATGAGTTGTCAAGTCTTCCAAGCAATCCAAGATGCTATA |
| | ATTTAATAGCATTGTTCCTTCAAAGAAACAGCTCCTTAAGGG |
| | TGATACCAACATCCTTTTTTGACAGCATGTCTACTCTTAAAA |
| | TTCTGAATCTCTCCAAAACCAGAATAAAGTCTTTACCTGATT |
| | CCTTTTCTAGACTCAAAAGCCTCCAAGTCTTACTGCTGCGAG |
| | ATTGTGAGCGCTTGCTTGTGCTTCCTTCATCAATTGGATTTCA |
| | TAAATCTCTGAAGGTGCTTGATCTTCAAGGTACCGAGGTAAC |
| | CCACTTGCCTGATAGTATTGGTAAATTAGAAAGCCTGAAAC |
| | AGTTGAGAGTTTCATTCTATGGATCCATTAATCGTTGTGAAT |
| | ACAATAAGTTGCCTTCAAAGTTGGTATCAGATGGAATCATTT |
| | CGAGCCTTAAGCTTAGAGAACTTGGTGTTTTTGTTTATCCTG |
| | GAGATCGACGTTGGACATTGAGTGCATCAGGTATCACGAAT |
| | GAGATCAGTAGCCTGAAGTTGTATGTGCTTTATTTTCACTTC |
| | CCAAAAGTAGAAAATCTCGAGCACTTTGTGCATACAAGCCA |
| | GTCGTGGAAGGCAAGAGAGTTGGCTAAATTCAATTTTATTGT |
| | TGGACATGATTTCAAACGTATTGTATCTTTGGTTTCAAATGA |
| | TGCAGAACTCTTGTACAACGAAGGTCAAAGATGTTTAAGAT |
| | TTGTGAATGGTGAAAGTATACCTGATCCGGTGGTAGAGGTA |
| | TTGACACGAGCTACTTCTTTTTACTTAGACCATCACCTGAAC |
| | ATCTCCAATCTATCACAATTTGGAATTGACTGCTTTGATGGC |
| | TTAAAATTTTGCATACTAAGGGATTGTCCGGAGCTGGTAGGA |
| | ATTTTGGATAGTCAAGACCAGCGAAGTGCATTCCTTCCTTTT |
| | CTAGAATACTTGAGTATCAACTACCTCTGGAACTTAAAAAG |
| | AATTTGGGTTGGTTATATTGCAGCAGGAAGTTTTGCTAGGTT |
| | AAGATGCTTATCGATTCATGCTTGTCCAAAATTGAGCTATGT |
| | TCTTACTTGCCCCATGCTTGATATTCTCCGTAATTTGGAGGA |
| | GTTGGTGATTGAAGACTGTGTATCCCTAAAATATGTTGTGAG |
| | TGAAGACGAGGATGTCAAAGAGATCAAGTATGCTGATGTAG |
| | ACAGTGTTTCTGCTCTTGAGTTGTCTCCGCTTCATATGTTGAA |
| | AGTATTAAAACTTCATTATCTACCAGAGATGTGCGGCATTTG |
| | GAGAACCCGGTGGCCTCCATTGGAGTACGTCAGCTTTTACGA |
| | TTGTCCCAAGCTCAGAAATCTTCATATGGAAGGGTATGATGA |
| | CACATTCATTAAAGAAATAGCAGCAGATAAAGGGTGGTGGG |
| | ATTCTTTAGAGTGGGACGATCTTGAACTGTCAAGAAGACTTC |
| | TCAAGCACGTGACCGAACTCCATGTTGATGAATTGTAAAGA |
| | ATAGATATATCAACATATATACAGGGGTAACCTCTGCAAAC |
| | CAATTTTGAATTTCTACAGACTGAGTTAAACAAAATTCAGTG |
| | GTTATACAACTGGTATGATCACTATGTTTATAGTGGGTGTTA |
| | TGACTCAACATTATTCTCTGTCCTAAATTTTTACAATACTAGT |
| | TTAGTACCTGTGCAACGCACGATGATTATTACTCCGTAAATA |
| | TATTTTTTTTGTAATACTTTTAATAATTATTTACATAAAAGTT |
| | AATAAAATATATTTTGTAAATGATTTCTTCATATAACAATTG |
| | ATTTAATTTTTTACCTAAATAAATAATTTTAAAAAATTATGTT |
| | TAATGATATAAATTGGATACTATCATTGAACTGCAATTCAAA |
| | ATTATACGTTAAAAATGTTTGATTTATTATATTGCATTATTTT |
| | AATTTCCCATAAAATTAATATATATACAAAACTTTCCAAAAT |
| | TTTTATTACTGTTGTGAAATACTCCCTCCGTCCCTTAATACTC |
| | ACACCACTTTCCTTTTCGGTTTTACTTTAATACTCGCACCGCT |
| | TCTATAAATGGAATTTATTTACCAATATTATATTATTTTTCAA |
| | ACTTACCTACTAATCCACTTGTACCCCTACTCCCTATTAAAA |
| | AAATCATTTAAGAATTCACACCCCCCACTACCCCTTCAGTGG |
| | CGGAACTACGTGAGTGACGTGACCCAGCGAAACTTTGAAAA |
| | AAAACCATAGGTATCTAAGATGTTATATCATAGAGCCGGTCT |
| | CTTATATTATACTAGAAAAACCTAATCCTGCACATGAGACAA |
| | CGGTTATAACTACCATATTTTGTGATAAATCTATAAGACTTG |
| | TTCTTTAAAGTAACATGTCACTTAAAATCGAGTAACAGACGG |
| | CCTGCTGTATTCAATTCAAATCTAAAGAACAGTTATTTAAAT |
| | ACCTGTTCTCTTAAATGGAGTTATTTATTTCTAACATCCACAT |
| | AAAAGCTGTAGAAAATAATTGAATAATTTTGTAGGTATTTGA |
| | TTGTAATATAAATATTTATTATTTTTGGGACGATATATATATT |
| | TTTTTAATATATATTGTGTGTTGTGGACGATGGTGACCCAAC |
| | ACCTAAACATCTTGGATACGCCACTGTTCCTCTTACACATTC |
| | TCCACTAACTATATTAAAAAAACACCCCACTATCAATTACCA |
| | CATATTAAATTAATATGTCAATTCAAGTGTAAACTCCGTACC |
| | GGTCAAATCGGTGCGAGTATTTTTTAAACCAAATTCGTGATG |
| | AATAACATGCATAACTTTTATTTGACAACAAAAATCACGTAC |
| | AAATTAGATTGTATACGGAGTAGTTACTTTTAACAAATTAAT |
| | TTCTTAACTTTTCGACTATGCATTTGTGATGAGATCTTCCAAT |
| | AGGTATAAACCATTGGTTAAAATACTGTTTGGAGAAAAAAA |
| | TAAAGAGGTATTCCGTCATCATTCTACGACAATAAATATTGA |
| | TTTATTAATGTTGGTTTACATGCATTTTAAAATCCATGCATAC |
| | ATCGCGTGCATAAAATACTACTCCGTAGTAAAAGATAAAAG |
| | GACGGAGGGACGTAGTAAAAGATAAAAGGACGGAGGGAGT |
| | AACATATACACCAGGGTATAACACGTGGACAATAAACGGAC |
| | GGTAACAGGCTAACACAGCTTGTCATCCATAAGGCTAACTTT |
| | CCATTTGTAGTCCGCGCTTGAAAATCAATCAAACTTTTGATG |
| | CTTCCCACCAAAAAAACACCCCAATATTTTTGAACCGAAAAT |
| | ATAAAAATTTCTGCTTGCCTTTCTATCACCTTGATCCATCTCT |
| | CTCTCCTCAATCCATCTTGTCGCTTCACATCTCTCTGATTTGA |
| | ACTTGAAATCAAAATTGATGATTGGTAAAAAGCGTCGTACT |
| | GTCTCATTGAGTTCTACCCCCGACGATAACTATGTGAGTCTG |
| | CTGATTTATCTCGCAATTTTTTTTTTTATTTACTTTGTATAAAT |
| | GTTCAATTACCCCTTG |
| SEQ ID NO: 12: | ACTAATTGCCAACATCTGATTTCCCGAATACAAATAAATTTC |
| genomic sequence | CATAACAGACCTCAACCCTAGATTATAATTTTGTTTTTAATTT |
| of the wildtype p10 | GTACTCCGTAATTTACAAGATAAAAGAAAATACATTTATATT |
| kinase gene not | ACTCCCTCCGTATTTATTTAAGAGATACACTTGGCCGGACAC |
| conferring | GGATATTAAGAAAAAGAATTAAATGAAATAAAGTAATAAAA |
| resistance as | CAAGTGAGGTTGAGTAGATATTTTAATAAGAAAAACAAGTG |
| present in Sp75, | GGGACCATGTCATTTTAGGGAGGGGAGAGTGGAAGTGGGGT |
| including 2 kb | GTAGATATATTATTTAAATAGATGGTGGGGTTGATAAGTTAC |
| upstream and 2 kb | TAAAAATGGCAAGTGTATCTCTTAAATAAATACGGCCGGAA |
| downstream | AAGGCAAGTGTATCCCTTAAATAAATACAGAGGGAGTATGA |
| | AAAGGAATTTATAAAAAAAAATGTTGTGAGATGAACCTAAA |
| | TTTTTTGTCAGAAAGGACCTCTTAAAGTCTGAAAATTATGAC |
| | ATTGATTTTTTGTTGTGAGATGAAACCTTTACCCATTTTTCGA |
| | TAAAAATTTTCCGGTTTTGACTCGTGTGTGTCGGTCATGTGC |
| | TTCTTAAAAATAATTTCAGATGAACCCCAATTTCCACAAAAG |
| | TCAAATTAAACGAATTGATGCGGAAATTGGGCCTTTTTCTCT |
| | TTCCTTGTTTGAATAGGGCGCAACGATTTGTTTTTTTGGTATC |
| | TTGCACAATTGAAGAACCGGTCGAAGAAGATGAACATGAGA |
| | GGGACCGTAGAGTTAGGAAGGCGCAATTTAGAAGGTGGACC |
| | ATGGTGCACCTTAAGAATATTAGTATATAATTAAAAGAACA |
| | TCTGGTTATGTAAAAGGAACATGGACATATATTTTTTTATAT |
| | TTTTAATAAATTGTGATTTTTATAACAAAAAAATAAAATATT |
| | ATATTGCATGTTCTTTAGTCGTAGTGTCATGTTCTTTTGTTTA |
| | TGCACATGTGTTCTTTTGGTGCACATGGTGCACCATGGTCCA |
| | TAGTCCACGTAAAAAAAATCATTTTTTAATAAGCACGTGACC |
| | AAAGCGCGCGAGTCAACGCCGAAAAATTGGAGTTACCATCG |
| | AAAAATTGATAAAGGTCTCGTCTCACAACAAAAAATCAATT |
| | TTAGGTCCTATCTCACAACAATTTTTTTTATAAGGTCATTTAT |
| | CACAATTTTTGGACTTTAAAAAAATTTCTAACAAAAAATCAT |
| | ACTTCCTCTATTTTTAATAATTGTACCATCTTGGTTTTAACAC |
| | TATTCCCATATTCTTATATAGTTATTTTTTTTGTAATTTATAT |
| | GTAAGGAAAAATATATTCATGTGGGATCTTGTTAGATTCGTC |
| | TCGAAATATACTTTCAAATTAGCAAATTTTTATAATTTTTTAA |
| | AATGTATAATGGGAGATATTAGTGGTTAAAATAGTGCATTG |
| | GCAAGCGTGAAATCAAAGATGGTACAATTAAAAAAAATGGA |
| | GGAAGTATATATTACTATAAATATTTGTTTCCATCTTTGTATC |
| | GACTATTTTAATTACATATTTCCATAGTAAAAGTTCATGCGA |
| | CACCATTAAGAATACGCCACATAAAATTGCGACAATTAAAA |
| | CTTATTGCAAGTTGCGACCTTTATCATCACCCTAGTTGTTTAT |
| | TTTTTCATGTAAAAAGAATAAAATAGGATAATGTTTGAGTGG |
| | TCTAGTGATGTCTGGGCTTGTTGTTTGTTTCCAATTTCAATCA |
| | AGATCCTCAAATTCTAACCACATTCAAACTTACATTAATGGT |
| | TGGCACTTGGAAGCTTCATTTCTCAACAATATTCAAAACGAA |
| | AACATTGTGGTAGTCGTTGTACCATTCAAAGACTCCAACTTT |
| | GTCAAAGTTGGCAGCTTCTTCTCTTATTCTTCATTTGTGAAAT |
| | GTCAAGTTCCCTTTGCGAACAAACAAACACAGCACAGCTAA |
| | TACCATTTCTCAACAAACACAGCACAGTATTGGATTGGTAAT |
| | TTAATTTGGGTGTATATTTACTATTGTTATGGGGGGTTGTTTG |
| | TCATATAGATTGAAGTTGATCCGCGATCTAAACCCAAATGA |
| | GGATACTTGGTTAGGAAACGTGACTAGTTTTTCCTACAAATC |
| | ACTGAAAAAGGCAACAAACAATTTCAGCAATAGGCTCAGAT |
| | CGAATGGATTTTCATCATCTTACAAGGGCACTTTAACAGATG |
| | GGTTACAAGTCATAGTTAACGTATATAGAACCGATTATAGTG |
| | TCATAGACTTGGCTTCAATAATCAAAATCATGTCTGGAGTCA |
| | GGCACCAAAATGTAGTGGAACTCATTGGGTACTGCATTGAA |
| | GGTAATCATAAAATGCTGGTGCACGAGTTTGTAGGGGATTG |
| | CAGCCTGGATCAGTTGTTGTTTCCAGACTCCAGCAGTGGGGT |
| | AGGAGATCGTTCTGTGCTAGATTGGCCCATGAGAGTGTCCAT |
| | ATGCTTTAAGATTGTTCGGGTTCTAGAATATCTTCACTGCGG |
| | GATCAGTCCCCGTGTTGTGCTTCGTGATTTCAAGCCTGGGAA |
| | TGTGTTAATGGATTCAAGTTTCAACCCCAAAATAGTAGGTTT |
| | TGAGTGTTCAAGTCTTCTAGAAGACAGTGAATTAGACCGTA |
| | GTAGCCGTATTTGTGGAACAATTGGCTACTTGGATATTGAAT |
| | ATTGTATAACAGGAAGATATTCAGAGAAGACAGATGTGTTT |
| | TCTTTTGGAATTACTCTACTCGAAATCATCAGTGGTAAGAAA |
| | AATCTGCGGTTTATCTTGGTGAATTGGGCTTGGGACCTACAA |
| | GCAAGTGGTAGGATTCCTGAGATTGTCGATCCTACTCTATCT |
| | CAAGTTTCAGAAGATGAAGTGATACGTTTTGTCAAAGTTGCG |
| | CTTTCGTGTGTCCAATATTCAGAACTCAGACCGAGCATGTCT |
| | GAGGTAAGGGTAATGCTCTCAGACGGATATGACTTCAGTAA |
| | CATGGTACTAACTAGACCATAGAGGAATAATTCTTTTGAGTT |
| | TTGACATTGATGAATGATATATGATCAACGTTGATGTGCCGT |
| | AAAAAAGGTAGAAAGAAAATGTACGTTTTAAGTAATACCTG |
| | CTTCAAACAAACCGACCACTCGCGTTGTATATCTCTACAATA |
| | ATGTTGTAATACTATAACTGTATTAACATATTGCACGCAGTA |
| | TGATTTAAGAACAACAGGACATCATTGATATCAATGATATG |
| | AGGAAAACCCAACTTTAAGAAACTTTTTACTTCAACTTTCAT |
| | TGCCTGTGTTGTATGGAATGTATTTTTTCATCTGTTTAGAATA |
| | ATATGTGTTATAATATTATTCTAATAATACGGAGTATGTGTT |
| | AGAATAATATGAAACAGGGTAACAATGTCTTATAATATGAA |
| | ATTTAACATTTATTATCAATAGGGGTGGTGGCGCAGTTGGCT |
| | AGCGCGTAGGTCTCATAGCTAAGATGAGTGATCCTGAGGTC |
| | GAGAGTTCGAGCCTCTCTCACCCCAACTGTAAATTTTTTAAA |
| | AGAAGCAACTTTTCTTTTTTAACCAGTATGCATTCTACATTCT |
| | CTACGCATTTGCTGTTTGCTGTCCATCTGTTCGACAATATGTC |
| | AAAGAACTGATTATATTTGTGTTTTAAACCAAGCTAAGCAGT |
| | TCAAGTTAACATTCTGACATGGCAAAGTAAACTCTACCAGA |
| | AATATGAAAAGAATACAATAAATTTTAGCCAATATTTAATAT |
| | CAGCACATCTTGCACATTTCAGTTATTGGTTTACAACATACA |
| | GTTATATTAAAAGTTGGGAACAACTGCAAGTCTGAATGTAA |
| | GTAAATACATAGAGTGAAAATGGAGTTATGAGGTGCCGGAA |
| | ATGGACATTAATATCTCGTCTTCACATTCTGCAAGAACATGA |
| | ACAACTGACTCCATGGTTGGCCTTTTATTTTTATCATCCTCCA |
| | CGCAAGAAAGTCCAACCTTAATCATTATACCAGCTTGGTATC |
| | TACTGAAGTTTCCTTCCAATCTCGGGTCTACAAAGTCTTCTA |
| | CCCATGAATCTTCTTCATTCTGCATTTTGCTTTTAGCCAATCT |
| | CACAATTTTTACCAGTTCTGATATATCCTCTAAGTCATTATG |
| | ATCATCCACCACCCTACTTGACAGTCGAATTCCCTTGACAAG |
| | CTCTAGGATAACAACTCCATAACTGTAGACATCAACCTTTGC |
| | AGTAATGGGTTGGTTCAATGCCCATTCAGGTGCCATATAACC |
| | TTTTGTTCCTCTGATTCCGGTCAGCTCTGCTGAACCCTGACTC |
| | CCTCTTTGACGTAGCTTTGCTAGCCCAAAGTCTGAAATTTTT |
| | GGCTCAAAATCCTCATCCAAGAGTATATTTTCAGGTTTCACA |
| | TCACAATGGATAACCCATTCTAGGCACTCATGGTGAAGGTA |
| | AGCTAATCCTTTAGCCGTGCCTATTGCAACTTTAAGCCGCTC |
| | TTTCCATCCAACAAGGGTTTGGGTGGAGAACAAGTGTCTGTC |
| | CAAAGATCCATTTTCCACAAGCTCATAAACCAAGAGCTTGTG |
| | TTTCCTTTCGGAACAAAAACCCCACATTCTTGCAAGGTTCAT |
| | ATGATTGATTTTACCAATTGTACTCACTTCTGCCCAAAATTCT |
| | TCTTCTCCTTGGAATATATTTTCTAGTTTCTTCACTGCAACTG |
| | CTCTCTCATCTGCCAGAATACCCTTGTAAACAGCACCAAACC |
| | CTCCCTTTCCTAGAACTTCCTTAAATTTCCCTGTTGCAGTCTT |
| | GAGCTCGTTGTAGCTAAAGCTCCTAAACTGATTTGAAATGGC |
| | ACGATATCCATCTTCCATAGAAGTTGAAAGACCATGCTTCTT |
| | ATAAAGAAACCACCAAGCTGCTACCAAAAGGACTAGTTCAA |
| | GAATACCAATGACAGAAGCAAATGCGTAGATATAAACCCAC |
| | TTGAATCTTTGAATGGTGGTGTCATATGTGATAGGCAATTCA |
| | ACTACTTTACTTTGCTCGTTCCCACATTCAAGACGAGACACA |
| | TCAAAGC |
| SEQ ID NO: 13: | ATTCTAACCACATTCAAACTTACATTAATGGTTGGCACTTGG |
| 558 bp sequence | AAGCTTCATTTCTCAACAATATTCAAAACGAAAACATTGTGG |
| that is deleted from | TAGTCGTTGTACCATTCAAAGACTCCAACTTTGTCAAAGTTG |
| the genomic | GCAGCTTCTTCTCTTATTCTTCATTTGTGAAATGTCAAGTTCC |
| sequence of the | CTTTGCGAACAAACAAACACAGCACAGCTAATACCATTTCTC |
| p10-kinase gene | AACAAACACAGCACAGTATTGGATTGGTAATTTAATTTGGGT |
| and its upstream | GTATATTTACTATTGTTATGGGGGGTTGTTTGTCATATAGATT |
| region as present in | GAAGTTGATCCGCGATCTAAACCCAAATGAGGATACTTGGT |
| NCIMB 42554 | TAGGAAACGTGACTAGTTTTTCCTACAAATCACTGAAAAAG |
| | GCAACAAACAATTTCAGCAATAGGCTCAGATCGAATGGATT |
| | TTCATCATCTTACAAGGGCACTTTAACAGATGGGTTACAAGT |
| | CATAGTTAACGTATATAGAACCGATTATAGTGTCATAGACTT |
| | GGCTTCAATAATCAAAATCATGTCTGGAGTCAGGCACCAAA |
| | ATGTAGTGGAACTC |
| SEQ ID NO: 14: | ATGGGGGGTTGTTTGTCATATAGATTGAAGTTGATCCGCGAT |
| 288 bp sequence | CTAAACCCAAATGAGGATACTTGGTTAGGAAACGTGACTAG |
| that is deleted from | TTTTTCCTACAAATCACTGAAAAAGGCAACAAACAATTTCAG |
| the coding | CAATAGGCTCAGATCGAATGGATTTTCATCATCTTACAAGGG |
| sequence of the | CACTTTAACAGATGGGTTACAAGTCATAGTTAACGTATATAG |
| p10-kinase gene as | AACCGATTATAGTGTCATAGACTTGGCTTCAATAATCAAAAT |
| present in NCIMB | CATGTCTGGAGTCAGGCACCAAAATGTAGTGGAACTC |
| 42554 | |
| SEQ ID NO: 15: | AGGTTTTGAGTGTTCAAGTCTTCTAGAAGACAGTGAATTAGA |
| 45 bp sequence that | CCG |
| is deleted from the | |
| coding sequence of | |
| the p10-kinase gene | |
| as present in | |
| NCIMB 42554 | |
| SEQ ID NO: 16: | MGGCLSYRLKLIRDLNPNEDTWLGNVTSFSYKSLKKATNNFSN |
| 106 amino acids of | RLRSNGFSSSYKGTLTDGLQVIVNVYRTDYSVIDLASIIKIMSGV |
| sequence that is | RHQNVVELIGYCIEGNHK |
| deleted from the | |
| protein sequence of | |
| the p10-kinase as | |
| present in NCIMB | |
| 42554 | |
| SEQ ID NO: 17: | GFECSSLLEDSELDR |
| 15 amino acids of | |
| sequence that is | |
| deleted from the | |
| protein sequence of | |
| the p10-kinase as | |
| present in NCIMB | |
| 42554 | |
| SEQ ID NO: 18: | CAAGATTTTCTAGTGGCAGACACCACGACAAGAAGGATTCC |
| Allele of marker | AAGTCAGCCAGCTACTGAATCAATCAGTAGGACRWMACGT |
| SORZ01 (p10 | GGTTGCGAAATAGAAATAGAAATAGAGAGTGACCTTGACA |
| NLR-kinase 6 bp | CACATTCTGGTGCATCAGAACAAATGGAGATTTCACATGATC |
| insertion marker) | |
| linked to resistance | |
| SEQ ID NO: 19: | CAAGATTTTCTAGTGGCAGACACCACGACAAGAAGGATTCC |
| Allele of marker | AAGTCAGCCAGCTACTGAATCAATCAGTAGGACRWMACGT |
| SORZ01 (p10 | GGTTGCGAAATAGAAATAGAGAGTGACCTTGACACACATTC |
| NLR-kinase | TGGTGCATCAGAACAAATGGAGATTTCACATGATC |
| wildtype) linked to | |
| susceptibility | |
| SEQ ID NO: 20: | TTCGTGATTTCAAGCCTGGGAATGTGTTAATGGATTCAAGTT |
| Allele of marker | TCAACCCCAAAATAGTCCCAAGCCGTATTTGTGGAACAATT |
| SORZ02 (p10- | GGCTACTTGGATATTGAATATTGTATAACAGGAAGATATTCA |
| kinase 45 bp | GAGAAGACAGATGTGTTTTCTTTTGGAATTACTCTACTCGAA |
| deletion) linked to | ATCATCAGTGGTAA |
| resistance | |
| SEQ ID NO: 21: | TTCGTGATTTCAAGCCTGGGAATGTGTTAATGGATTCAAGTT |
| Allele of marker | TCAACCCCAAAATAGTAGGTTTTGAGTGTTCAAGTCTTCT |
| SORZ02 (p10 | AGAAGACAGTGAATTAGACCGTAGTAGCCGTATTTGTGGA |
| kinase wildtype) | ACAATTGGCTACTTGGATATTGAATATTGTATAACAGGAAG |
| linked to | ATATTCAGAGAAGACAGATGTGTTTTCTTTTGGAATTACTCT |
| susceptibility | ACTCGAAATCATCAGTGGTAA |
| SEQ ID NO: 22: | GATGCTCAGCCGCTCACCAGTATTTGGTTTTCATGAGCCAAA |
| Allele of flanking | AACTGGA |
| marker SO00979 | |
| SEQ ID NO: 23: | GATGCTCAGCCGCTCACCAGTATCTGGTTTTCATGAGCCAAA |
| Allele of flanking | AACTGGA |
| marker SO00979 | |
| SEQ ID NO: 24: | CATAAACATTCCGTATGAGTAGTACTCTATTTGTCTCAAAAA |
| Allele of flanking | GAAAATTGAAAATTGCCCTAGTCGAAATTTTATCTGCACTA |
| marker SO01770 | |
| SEQ ID NO: 25: | CATAAACATTCCGTATGAGTAGTACTCTATTTGTCTCAAAAA |
| Allele of flanking | GAAAATTGAAAATTGCCTAGTCGAAATTTTATCTGCACTA |
| marker SO01770 | |
| SEQ ID NO: 26: | ATGGCCGAAATCGGATACTCGGTTTGTGCGAAACTCATCGA |
| Coding sequence of | AGTGATTGGCAGTGAGCTGATCAAAGAGATTTGTGACACAT |
| the alpha-WOLF | GGGGTTACAAATCTCTTCTTGAGGACCTCAACAAAACTGTAT |
| 29 allele | TGACGGTCAGGAACGTTCTCATTCAAGCCGGGGTGATGCGG |
| | GAGCTTACTAGTGAACAACAAGGTTTCATTGCAGACCTTAA |
| | AGATGTTGTTTATGATGCTGATGACTTGTTCGACAAGTTACT |
| | CACTCGTGCTGAGCGAAAACAGATTGATGGAAACGAAATCT |
| | CTGAAAAGGTACGTCGTTTCTTTTCCTCTAGTAACAAGATCG |
| | GTCAAGCTTACTACATGTCTCGTAAGGTTAAGGAAATTAAG |
| | AAGCAGTTGGATGAAATTGTTGATAGGCATACAAAATTTGG |
| | GTTTAGTGCCGAGTTTATACCTGTTTGTAGGGAAAGGGGGA |
| | ACGAGAGGGAAACACGTTCATATATAGATGTCAAGAATATT |
| | CTTGGGAGGGATAAAGATAAGAATGATATCATAGATAGGTT |
| | GCTTAATCGTAATGGTAATGAAGCTTGTAGTTTCCTGACCAT |
| | AGTGGGAGCGGGAGGATTGGGAAAAACTGCTCTTGCACAAC |
| | TTGTGTTCAATGATGAAAGGGTCAAAATTGAGTTCCATGATT |
| | TGAGGTATTGGGTTTGTGTCTCTGATCAAGATGGGGGCCAAT |
| | TTGATGTGAAAGAAATCCTTTGTAAGATTTTAGAGGTGGTTA |
| | CTAAGGAGAAAGTTGATAATAGTTCCACATTGGAATTGGTA |
| | CAAAGCCAATTTCAAGAGAAGTTAAGAGGAAAGAAGTACTT |
| | CCTTGTTCTTGATGATGTATGGAACGAAGATCGTGAGAAGTG |
| | GCTTCCTTTGGAAGAGTTGTTAATGTTGGGTCAAGGGGGAA |
| | GCAAGGTTGTAGTGACCACACGTTCAGAGAAGACAGCAAAT |
| | GTCATAGGGAAAAGACATTTTTATACACTGGAATGTTTGTCA |
| | CCAGATTATTCATGGAGCTTATTTGAAATGTCGGCTTTTCAG |
| | AAAGGGCATGAGCAGGAAAACCATCACGAACTAGTTGATAT |
| | TGGGAAAAAGATTGTTGAAAAATGTTATAACAATCCACTTG |
| | CTATAACGGTGGTAGGAAGTCTTCTTTATGGAGAGGAGATA |
| | AGTAAGTGGCGGTCATTTGAAATGAGTGAGTTGGCCAAAAT |
| | TGGCAATGGGGATAATAAGATTTTGTCGATATTGAAGCTCA |
| | GTTACTACAATCTTGCAAACTCTTTGAAGAGTTGTTTTAGTT |
| | ATTGTGCAGTATTTCCCAAGGATCATAAAATAGAGAAGGAG |
| | ATGTTGATTGACCTTTGGATAGCACAAGGATATGTTGTGCCG |
| | TTGGATGGAGGTCAAAGTATAGAAGATGCTGCCGAGGAACA |
| | TTTTGTAATTTTGTTACGAAGGTGTTTCTTTCAAGATGTAGTG |
| | AAGGATGAATACGGTGATGTTGATTCTGTTAAAATCCACGA |
| | CTTGATGCACGATGTCGCCCAAGAAGTGGGCAGAGAGGAAA |
| | TCTGTATAGTGAATGATAATACAAAGAACTTGGGTGATAAA |
| | ATCCGTCATGTACATCGTGATGTCATTAGATATGCACAAAGA |
| | GTCTCTCTGTGTAGCCATAAGATTCGTTCGTATATTGGTGGT |
| | AAATGTGAAAAACGTTGGGTGGATACACTAATAGACAAGTG |
| | GATGTGTCTTAGGATGTTGGACTTGTCAAATTCAGATGTTAA |
| | AAGTTTGCCTAATTCAATAGGTAAGTTGTTGCACTTACGGTA |
| | TCTTAACCTGTCAAATAATAGAAATCTAAAGATACTTCCTGA |
| | TGCAATTACAAGACTGCATAACTTGCAGACACTACTTTTAGA |
| | AGATTGCAGAAGTTTAAAGGAGTTGCCAAAAGATTTTTGCA |
| | AATTGGTCAAACTGAGGCACTTGGATTTAAGGTTTTGTTCTG |
| | ATTTGATTGGTATGCCATTGGGAATGGATAGGCTAACTAGTC |
| | TTAGAGTACTGCCATTCTTTGTGGTGGGTAGGAAGGAACAA |
| | AGTGTTGATGATGAGCTGAAAGCCCTAAAAGGCCTCACCGA |
| | GATAAAAGGCTCCATTCGTATTAGAATCCATTCAAAGTATAG |
| | AATAGTTGAAGGCATGAATGACACAGGAGGAGCTGGGTATT |
| | TGAAGAGCATGAAACATCTCACGAGGGTTATTATTAGATTTG |
| | ATGATAAAGAAGGTGGATGTGTTAACCCTGAAGCTGTGTTG |
| | GCAACCCTAGAGCCACCTTCAAATATCAAGAGCTTATCTATA |
| | GATAATTACGATGGTACAACAATTCCAGTATGGGGAAGAGC |
| | AGAGATTAATTGGGCAATCTCCCTCTCACATCTTGTCGACAT |
| | CCAGCTTTGGTGTTGTAGTAATTTGCAGGAGATGCCAGTGCT |
| | GAGTAAACTGCCTCATTTGAAATCACTGTATCTTTATACTTT |
| | GATTAGCTTAGAGTACATGGAGAGTAGAAGCAGCAGCAGTA |
| | GCAGTGACACAGAAGCAGCAACACCAGAATTACCAACATTC |
| | TTCCCTTCCCTTGAAAAACTTACACTTTGGTATCTGGAAAAG |
| | TTGAAGGGTTTTGGGAACAGGAGACCGAGTAGTTTTCCCCG |
| | CCTCTCTAAATTGGAAATCTGGGAATGCCCAGATCTAACGTG |
| | GTTTCCTCCTTGTCCAAGCCTTAAAACGTTGAAATTGGAAAA |
| | AAACAATGAAGCGTTGCAAATAATAGTAAAAATAACAACAA |
| | CAAGAGGTAAAGAAGAAAAAGAAGAAGACAAGAATGCTGG |
| | TGTTGGAAATTCACAAGATGATGACAATGTCAAATTACGGA |
| | AGGTGGAAATAGACAATGTGAGTTATCTCAAATCACTGCCC |
| | ACAAATTGTCTTACTCACCTCAAAATAACTGGAATAGATTAC |
| | AGGGAGGGGGAGATTGAATCAGATTCCGTGGAGGAGGAGA |
| | TTGAATTGGAAGTTGGGGAGGCATTTCAGAAGTGTGCATCTT |
| | CTTTGAGAAGCCTCATCATAATCGGAAATCACGGAATAAAT |
| | AAAGTGATGAGACTGTCTGGAAGAACAGGGTTGGAGCATTT |
| | CACTCTGTTGGACTCACTCAAACTTTCAAATATAGAAGACCA |
| | GGAAGATGAGGGCGAAGACAACATCATATTCTGGAAATCCT |
| | TTCCTCAAAACCTCCGCAGTTTGGAAATTGAAAACTCTTACA |
| | AAATGACAAGTTTGCCCATGGGGATGCAGTACTTAACCTCCC |
| | TCCAAACCCTCTATCTACACCATTTTTATGAATTGAATTCCCT |
| | TCCAGAATGGATAAGCAGCTTATCATCTCTTCAATACCTGCG |
| | CATATACTACTGTCCAGCCCTGAAATCACTACCAGAAGCAAT |
| | GCGGAACCTCACCTCCCTTCAGACACTTGGGATATCGGATTG |
| | TCCAGACCTAGTTAAAAGATGCAGAAAACCCAACGGCAAGG |
| | ACTATCCCAAAATTCAACACATCCCCAAAATTGAGTCAGGA |
| | AGGATATGGATTGATGCAGCTTTCACAGGCCAACCTTTTACT |
| | GGGATTTGA |
| SEQ ID NO: 27: | ATGGCCGAAATCGGAATCTCGATTGCTGCAAAACTCATTGA |
| Coding sequence of | AGTTTTGGGCAGTAAGATCATCAAAGAGATTTGTGACATGT |
| the alpha-WOLF | GGGGTTACAATTCTCATCTTGAAGACCTCAACAAATCTGTCT |
| 30 allele splice | TGACGATCAAGGATGTGCTCTTGGATGCTGAGGCGAAGCGG |
| variant 1 | GATCTTTCCCGTGAACAACAGAGTTACATTGCAGAACTTAAG |
| | GATGTTGTTTATGATGCTGATGATTTGTTCGATGAGTTCCTC |
| | ACTCTTGCTGAGCTCAAACAGATTGATGGCAACTACAAGGG |
| | TGGTGGTAAATTCTCTGAAAAGGTACGTCGTTTCTTTTCTTCT |
| | AATAAGGAGAAGATGGATCAAGCTTACGACATGTCTCGTAA |
| | GGTTAAGAAAATTAAGAAGCAGTTGGATGAAATTGTTGATA |
| | GGCATACAAAATTTGGGTTTATTGTTGATTATAAACCTATTA |
| | TTAGGAGAAGGGAGGAAACATGTTCTTACGTAGATGCCAAG |
| | GAGATTATCGGGAGAGATAAGGATAAGGATGCTATCATTGA |
| | TATGTTGCTAGATCGTAATGATAAGGAGGGTTGTAGTTTTCT |
| | GACCATTGTGGGAGTTGGAGGGTTGGGGAAAACTGCTCTTG |
| | CCCAACTTGTGTATAATGATGAAAAGGTCATAAAAGAGTTC |
| | GAGGGTTTGAGGTATTGGGCTTGTGTCTCTGATCAAGACGGG |
| | GAGGAATTTGATGTGAAAGCAATCCTTTGTAAGATTCTAGA |
| | ATCAGTTACTAAGGTGAAACCTGATGGTAGTTCCGGATTGG |
| | AATTGGTGCAAAACCAATTTCAGGAGAGATTAAGGGGAAAG |
| | AAGTACCTCCTTGTTCTTGATGATGTATGGAATGAGGACCGT |
| | GAGAAGTGGCTTTCTTTGAAAAAGTTCTTAATGTTAGGTCAA |
| | AGGGGAAGCAGGATTATGGTAACCACTCGTTCTAAGACGAC |
| | GACAACCATCATAGGGGATAAACATGCCTATGAATTACAAG |
| | GTTTATCCCAAGAGGATTCATGGCACTTGTTTGAGATTTCTG |
| | CATTTGACAATGAATGTATCCGCAATAATGAGTTAGTTGAGA |
| | TTGGGAAGAAGATTGTTGAAAAATGTTATAGCATTCCTCTTG |
| | CTATAAAGGTGGTAGGAAGTCTTCTATTTGGCCAGGAGAAA |
| | GTTAAGTGGCAGTCATTTGAAGCGAGTGGATTGTCCCAAATT |
| | GGCAATGGTGATAATCAGATTATGTCAATATTAAAGCTCAGT |
| | TACCATAATCTTATACCCTCGTTGAAGAGTTGCTTCAGTTAT |
| | TGTGCAGTGTTTCCCAAGGATCATGAAATAAAGAAGGAGAT |
| | GTTGATTTATCTTTGGATAGCACAAGGATACGTTGTGGCACT |
| | TGATGGAGGTCAAAGTATAGAAGATGCTGCCGAAGAACATT |
| | TTGTAATTTTGTTACGGAGATGTTTCTTTCAAGATGTAAAGA |
| | AGGATTTTCTTGGTGATGTTGATTCTGTTAAAATCCACGACT |
| | TGATGCACGACGTCGCTCAAGAAGTAGGGAGAGAGGAAATC |
| | TGTATAGTGAATGCTAATACAAAGAACTTGGGTGATAAAAT |
| | CCGTCATGTACATTGTGATGTCAATAGATATGCACAAAGAGT |
| | CTCTCTGTGTAGCCATAAGATTCGTTCGTATATTGGTGGTAA |
| | ATGTGAAAAACGTTGGGTGGATACACTAATAGACAAGTGGA |
| | TGTGTCTTAGGGTGTTGGACTTGTCAAGGTCGGATGTTAAAA |
| | ATTTGCCTAATTCAATAGGTAAGCTGCTGCACTTACGGTATC |
| | TTAACCTGTCAAATAATAGAAATCTAAAGATACTTCCTGATG |
| | CAATTACAAGACTGCATAATTTGCAGACACTACTTTTACATC |
| | GGTGCAGTAGTTTAAAGGAGTTGCCAAAAGATTTTTGCAAA |
| | TTGGTCAAACTGAGGCACTTGGATTTATCGGGTTGTGAGGAT |
| | TTGATTGGTATGCCATTGGGAATGGATAGGCTAACTAGTCTT |
| | AGAGTACTGCCATTCTTTGTGGTGGGTAGGAAGGAACAAAG |
| | TGTTGATGATGAGCTGAAAGCCCTAAAAGGCCTCACCGAGA |
| | TAAAAGGCTCCATTGATATCAGAATCTATTCAAAGTATAGA |
| | ATAGTTGAAGGCATGAATGACACAGGAGCTGGGTATTTGAA |
| | GAGCATGAAACATCTCACTGGGGTTAATATTAGATTTGATGA |
| | TATAGAAGGTGGATGTGTTAACCCTGAAGCTGTGTTGGAAA |
| | CCCTAGAGCCACCTTCAAATATCAAGAGGTTAGAGGTGTGG |
| | CATTACGATGGTACAACAATTCCAGTATGGGGAAGAGCAGA |
| | GATTAATTGGGCAATCTCCCTCTCACATCTTGTCGACATCAC |
| | GCTTTTAGGTTGTAGTAATTTGCAGGAGATGCCAGTGCTGAG |
| | TAAACTGCCTCATTTGAAATCACTGTATCTTTTTAAGTTTTGT |
| | AAGTTAGAGTACATGGAGAGTAGAAGCAGCAGCAGTAGCA |
| | GTGACACAGAAGCAGCAACACCAGAATTACCAACATTCTTC |
| | CCTTCCCTTAAAAAACTTACACTTGGCGGTCTGAAAAAGTTG |
| | AAGGGTCTGGGGAACAGGAGATCGAGTAGTTTTCCCCGCCT |
| | CTCTAAATTGGTAATCTGGGAATGCCCAGATCTAACGTGGTT |
| | TCCTCCTTGTCCAAGCCTTGAAAAGTTGAAATTGGAAAAAA |
| | ACAATGAAGCGTTGCAAATAATAGTAAAAATAACAACAACA |
| | AGAGGTAAAGAAGAAAAAGAAGAAGACAAGAATGCTGGTG |
| | TTGGAAATTCACAAGATGATGACAATGTCAAATTACGGAAG |
| | GTGATAATAGACAATCTGGGTTATCTCAAATCACTGCCCACA |
| | AATTGTCTTACTCACCTCGACCTTACAATAAGAGATTCCAAG |
| | GAGGGGGAGGGTGAATGGGAAGTTGGGGAGGCATTTCAGA |
| | AGTGTGTATCTTCTTTGAGAAGCCTCACCATAATCGGAAATC |
| | ACGGAATAAATAAAGTGATGAGACTGTCTGGAAGAACAGGG |
| | TTGGAGCATTTCACTCTGTTGGACTCACTCAAACTTTCAAAT |
| | ATAGAAGACCAGGAAGATGAGGGCGAAGACAACATCATATT |
| | CTGGAAATCCTTTCCTCAAAACCTCCGCAGTTTGGAAATTGA |
| | AGACTCTGACAAAATGACAAGTTTGCCCATGGGGATGCAGT |
| | ACTTAACCTCCCTCCAAACCCTCGAACTATCATATTGTGATG |
| | AATTGAATTCCCTTCCAGAATGGATAAGCAGCTTATCATCTC |
| | TTCAATACCTGGGCATATTCAACTGTCCAGCCCTGAAATCAC |
| | TACCAGAAGCAATGCGGAACCTCACCTCCCTTCAGACACTTG |
| | GGATATCGGATTGTCCAGACCTAGTTAAAATATGCAGAAAA |
| | CCCAACGGCGAGGACTATCCCAAAATTCTATACATCCCCGG |
| | CATTATTAGAGGGTAG |
| SEQ ID NO: 28: | ATGGCCGAAATCGGATACTCGGTTTGTGCGAAACTCATCGA |
| Coding sequence of | AGTGATTGGCAGTGAGCTGATCAAAGAGATTTGTGACACAT |
| the alpha-WOLF | GGGGTTACAAATCTCTTCTTGAGGACCTCAACAAAACTGTAT |
| 24 allele | TGACGGTCAGGAACGTTCTCATTCAAGCCGGGGTGATGCGG |
| | GAGCTTACTAGTGAACAACAAGGTTTCATTGCAGACCTTAA |
| | AGATGTTGTTTATGATGCTGATGACTTGTTCGACAAGTTACT |
| | CACTCGTGCTGAGCGAAAACAGATTGATGGAAACGAAATCT |
| | CTGAAAAGGTACGTCGTTTCTTTTCCTCTAGTAACAAGATCG |
| | GTCAAGCTTACTACATGTCTCGTAAGGTTAAGGAAATTAAG |
| | AAGCAGTTGGATGAAATTGTTGATAGGCATACAAAATTTGG |
| | GTTTAGTGCTGAGTTTATACCTGTTTGTAGGGGAAGGGGAAA |
| | CGAGAGGGAAACACGTTCATATATAGATGTCAAGAATATTC |
| | TTGGGAGGGATAAAGATAAGAATGATATCATAGATAGGTTG |
| | CTTAATCGTAATGGTAATGAAGCTTGTAGTTTCCTGACCATA |
| | GTGGGAGCGGGAGGATTGGGAAAAACTGCTCTTGCACAACT |
| | TGTGTTCAATGATGAAAGGGTCAAAATTGAGTTCCATGATTT |
| | GAGGTATTGGGTTTGTGTCTCTGATCAAGATGGGGGCCAATT |
| | TGATGTGAAAGAAATCCTTTGTAAGATTTTAGAGGTGGTTAC |
| | TAAGGAGAAAGTTGATAATAGTTCCACATTGGAATTGGTAC |
| | AAAGCCAATTTCAAGAGAAGTTAAGAGGAAAGAAGTACTTC |
| | CTTGTTCTTGATGATGTATGGAACGAGGATCGTGAGAAGTG |
| | GCTTCCTTTGGAAGAGTTGTTAATGTTGGGTCAAGGGGGAA |
| | GCAAGGTTGTAGTGACCACACGTTCAGAGAAGACAGCAAAT |
| | GTCATAGGGAAAAGACATTTTTATACACTGGAATGTTTGTCA |
| | CCAGATTATTCATGGAGCTTATTTGAAATGTCGGCTTTTCAG |
| | AAAGGGCATGAGCAGGAAAACCATCACGAACTAGTTGATAT |
| | TGGGAAAAAGATTGTTGAAAAATGTTATAACAATCCACTTG |
| | CTATAACGGTGGTAGGAAGTCTTCTTTATGGAGAGGAGATA |
| | AGTAAGTGGCGGTCATTTGAAATGAGTGAGTTGGCCAAAAT |
| | TGGCAATGGGGATAATAAGATTTTGCCGATATTAAAGCTCA |
| | GTTACCATAATCTTATACCCTCGTTGAAGAGTTGTTTTAGTT |
| | ATTGTGCAGTGTTTCCCAAGGATCATGAAATAAAGAAGGAG |
| | ATGTTGATTGAACTTTGGATGGCACAAGGATATGTTGTGCCG |
| | TTGGATGGAGGTCAAAGTATAGAAGATGCTGCCGAGGAACA |
| | TTTTGTAATTTTGTTACGAAGGTGTTTCTTTCAAGATGTAAA |
| | GAAGGATAAATATGGTGATGTTGATTCTGTTAAAATCCACG |
| | ACTTGATGCACGATGTCGCCCAAGAAGTGGGGAGGGAGGAA |
| | TTATGTGTAGTGAATGATAATACAAAGAACTTGGGTGATAA |
| | AATCCGTCATGTACATCGTGATGTCATTAGATATGCACAAAG |
| | AGTCTCTCTGTGTAGCCATAGCCATAAGATTCGTTCGTATAT |
| | TGGTGGTAATTGTGAAAAACGTTGTGTGGATACACTAATAG |
| | ACAAGTGGATGTGTCTTAGGATGTTGGACTTGTCAAGGTCGG |
| | ATGTTAAAAATTTGCCTAATTCAATAGGTAAATTGTTGCACT |
| | TGAGGTATCTTAACCTGTCAGATAATAGAAATCTAAAGATA |
| | CTTCCTGATGCAATTACAAGACTGCATAATTTGCAGACACTT |
| | CTTTTAGTAGATTGCAGAAGTTTAAAGGAGTTGCCAAAAGA |
| | TTTTTGCAAATTGGTCAAACTGAGGCACTTGGAATTACAGGG |
| | TTGTCATGATTTGATTGGTATGCCATTTGGAATGGATAAGCT |
| | AACTAGTCTTAGAGTACTACCAAAAGTTGTGGTGGGTAAGA |
| | AGGAACAAAGTGATGATCAGCTGAAAGCCCTAAAAGGCCTC |
| | ACCGAGATAAAAGGCTCCATTGATATCACAATCTATTCAAA |
| | GTATAGAATAGTTGAAGGCATGAATGACACAGGAGGAGCTG |
| | GGTATTTGAAGAGCATGAAACATCTCACGGGGGTTGATATT |
| | ACATTTTTGGGTGAATGTGTTAACCCTGAAGCTGTGTTGGCA |
| | ACCCTAGAGCCACCTTCAAATATCAAGAGCTTATCTATACAT |
| | CGTTTTGATGGTAAAACACTTCCAGTATGGGGAAGAGCAGA |
| | GATTAATTGGGCAATCTCCCTCTCACATCTTGTCGACATCAA |
| | GCTTAGTTGTTGTCGTAATTTGCAGGAGATGCCAGTGCTGAG |
| | TAAACTGCCTCATTTGAAATCACTGGAACTTCGATGTTTGGA |
| | AAACTTAGAGTACATGGAGAGTAGAAGCAGCAGCAGTAGCA |
| | GTGACAAAGAAGCAGCAACACCAGAATTACCAACATTCTTC |
| | CCTTCCCTTGAAAAACTTACACTTTGGTATCTGGAAAAGTTG |
| | AAGGGTTTGGGGAACAGGAGATCGAGTAGTTTTCCCCGCCT |
| | CTCTAAATTGGAAATCCGGGAATGCCCAGATCTAACGTGGTT |
| | TCCTCCTTGTCCAAGCCTTGAAACGTTGAAATTGGAAAAAAA |
| | CAATGAAGCGTTGCAAAAAATAACAACAACAAGAGGTAAA |
| | GAAGAAAAAGAAGAAGACAAGAATGCTGGTGTTGGAAATT |
| | CACAAGATGATGACAATGTCAAATTACGGAAGGTGAAAATA |
| | GACAATCTGGGTTATCTCAAATCACTGCCCACAAATTGTCTT |
| | ACTCACCTCGACCTTACAATAAGAGATTCCAAGGAGGGGGA |
| | GGGTGAATGGGAAGTTGGGGATGCATTTCAGAAGTGTGTAT |
| | CTTCTTTGAGAAGCCTCACCATAATCGGAAATCACGGAATA |
| | AATAAAGTGAAGAGACTGTCTGGAAGAACAGGGTTGGAGCA |
| | TTTCACTCTGTTGGACTCACTCAAATTTTCAAAGATAGAAGA |
| | CCAGGAAGATGAGGGCGAAGACAACATCATGTTCTGGAAAA |
| | CCTTTCCTCAAAACCTCCGCAGTTTGAGAATTAAAGACTCTG |
| | ACAAAATGACAAGTTTGCCCATGGGGATGCAGTACTTAACC |
| | TCCCTCCAAACCCTCTATCTACACCATTGTTATGAATTGAAT |
| | TCCCTTCCAGAATGGATAAGCAGCTTATCATCTCTTCAATCC |
| | CTGTACATATACAAATGTCCAGCCCTAAAATCACTACCAGA |
| | AGCAATGCGGAACCTCACCTCCCTTCAGAGCCTTGTGATACG |
| | GCGGTGTCCAGACCTAATTGAAAGATGCGAAGAACCCAACG |
| | GCGAGGACTATCCCAAAATTCGACACATCTCCAGAATTGTA |
| | CTAAATGAATATTGGTGA |

### Marker Information

Marker SORZ01 was designed on the 6 bp insertion present in the allele of the p10 NLR-kinase gene as found in the p10-line that is absent in susceptible material such as Viroflay and Sp75. Marker SORZ02 was developed on the 45 bp deletion present in the coding sequence of the allele of the p10-kinase gene as present in the p10-line.

SEQ ID NO: 18 and SEQ ID NO: 20 represent the alleles of markers SORZ01 and SORZ02, respectively, that indicate the presence of the nucleic acid molecule of the invention. Therefore, the homozygous presence of SEQ ID NO:18 and SEQ ID NO: 20 is linked to the resistance conferred by the p10 locus, which is an at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

The sequences of SEQ ID NO: 19 and SEQ ID NO: 21 represent the wildtype alleles for markers SORZ01 and SORZ02, respectively, as present in spinach plants not comprising the nucleic acid molecule of the invention.

For markers SORZ01 and SORZ02 the nucleotides that are different between the marker allele indicating the presence of the nucleic acid molecule of the invention and the marker allele linked to the susceptible allele in a plant of variety Viroflay are underlined and in bold in Table 1. For SORZ01 this difference is an INDEL, wherein SEQ ID NO: 18 on positions 87-94 has sequence CGAAATAG and SEQ ID NO: 19 on positions 87-88 has sequence CG. For SORZ02 this difference is an INDEL, wherein SEQ ID NO: 20 on positions 58-63 has sequence TCCCAA and SEQ ID NO: 21 on positions 56-108 has sequence TAGGTTTTGAGTGTTCAAGTCTTCTAGAAGACAGTGAATTAGACCGTAGTA (SEQ ID NO: 29).

Marker alleles SEQ ID NO: 18 and SEQ ID NO: 17 may be used as molecular markers for detecting the presence of the nucleic acid molecule of the invention in the progeny of a cross between a plant of fully susceptible reference variety Viroflay and a plant comprising the p10 locus, which plant may be a plant grown from seeds of which a representative sample was deposited with the NCIMB under NCIMB accession number 42554.

The nucleotides that are different between the two marker alleles of markers SO1770, and SO00979 are underlined and in bold in Table 1. In the case of marker SO01770 the marker allele (SEQ ID NO: 25) has a single nucleotide deletion at the position where marker allele (SEQ ID NO: 24) has a C that is underlined and in bold (position 58 of SEQ ID NO: 21). For SO00979 this is a SNP on position 24, wherein SEQ ID NO: 22 on position 24 has a T and SEQ ID NO: 23 on position 24 has a C.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1—Bioassay for Resistance to Peronospora effusa in Spinach Plants with the p10-Locus

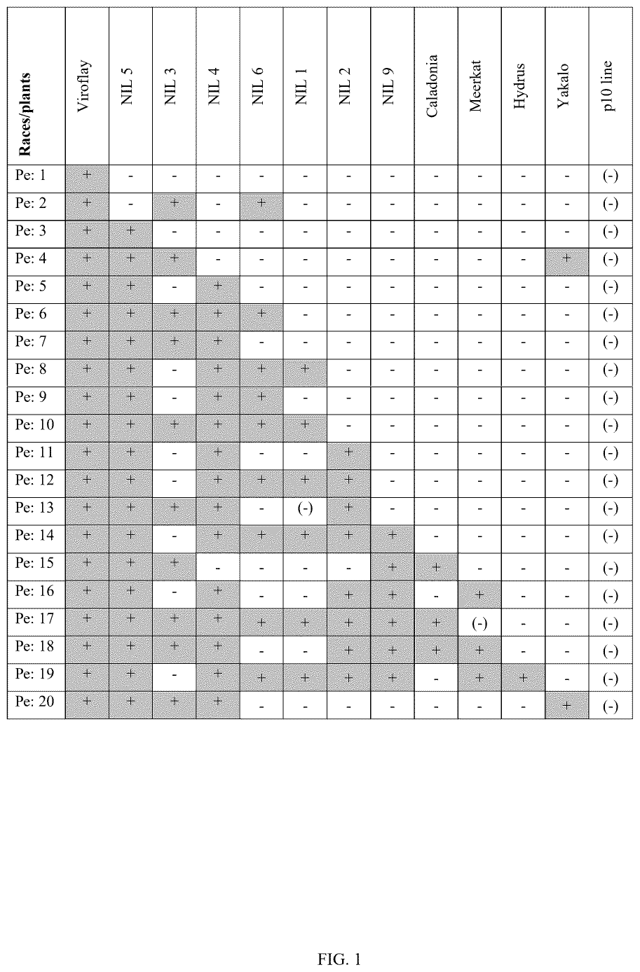

The resistance to downy mildew infection was assayed in a seedling test as described by Irish et al. (2008, Phytopathology 98: 894-900), using the differential set of the International Seed Federation (https://worldseed.org/document/isf-different-set-spinach-update-2024/). Spinach plants of the invention comprising the p10-locus homozygously were planted along with spinach plants different other genotypes (see FIG. 1) in trays containing Scotts Redi-Earth medium and fertilized twice a week after seedling emergence with Osmocote Peter's (13-13-13) fertilizer (Scotts). Plants were inoculated with a sporangial suspension (2.5×10⁵/ml) of a pathogenic race of Peronospora effusa at the first true leaf stage. In this manner, 20 officially recognized pathogenic races were tested (as shown in FIG. 1).

The inoculated plants were placed in a dew chamber at 18° C. with 100% relative humidity for a 24 h period and then moved to a growth chamber at 18° C. with a 12 h photoperiod for 6 days. After 6 days, the plants were returned to the dew chamber for 24 h to induce sporulation, and they were scored for disease reaction.

Plants for this specific test were scored as resistant, intermediately resistant, or susceptible based on symptoms of chlorosis and signs of pathogen sporulation on the cotyledons and true leaves, as described by Irish et al. (2007, Plant Dis. 91: 1392-1396). Plants exhibiting no evidence of chlorosis and sporulation were in this specific test considered as resistant. Resistant plants were re-inoculated to assess whether plants initially scored as resistant had escaped infection, or whether they were truly resistant. Plants that showed only symptoms of chlorosis, or sporulation occurring only on the tips of the cotyledons were scored as intermediately resistant. Plants showing more than these symptoms of downy mildew infection were scored as being susceptible.

FIG. 1 shows the 20 officially recognized Peronospora effusa races and the resistance of spinach plants of the p10-line and of the differential set to each one of these pathogenic races. A susceptible reaction is scored as “+” (indicating a successful infection by the fungus, with sporulation occurring on the entire cotyledon), and resistance is depicted as “−” (absence of sporulation on the cotyledons). A weak resistance response is indicated as “(−)”, which in practice means a slightly reduced level of infection (with only symptoms of chlorosis, or sporulation only occurring on the tips of the cotyledons in the differential seedling test). The p10 line in FIG. 1 is a line which is homozygous for the p10-locus and exhibits the broad-spectrum resistance of the present invention. Plants heterozygously comprising the p10-locus are susceptible to all 20 officially recognized Peronospora effusa races.

In FIG. 1, a susceptible reaction is scored as “+” (indicating a successful infection by the fungus, with sporulation occurring on the entire cotyledon), and resistance is depicted as “−” (absence of sporulation on the cotyledons). A weak resistance response is indicated as “(−)”, which in practice means a slightly reduced level of infection (with only symptoms of chlorosis, or sporulation only occurring on the tips of the cotyledons in the differential seedling test).

### Example 2—Identification of Candidate Genes Conferring the Non R-Gene Mediated Resistance of the Invention

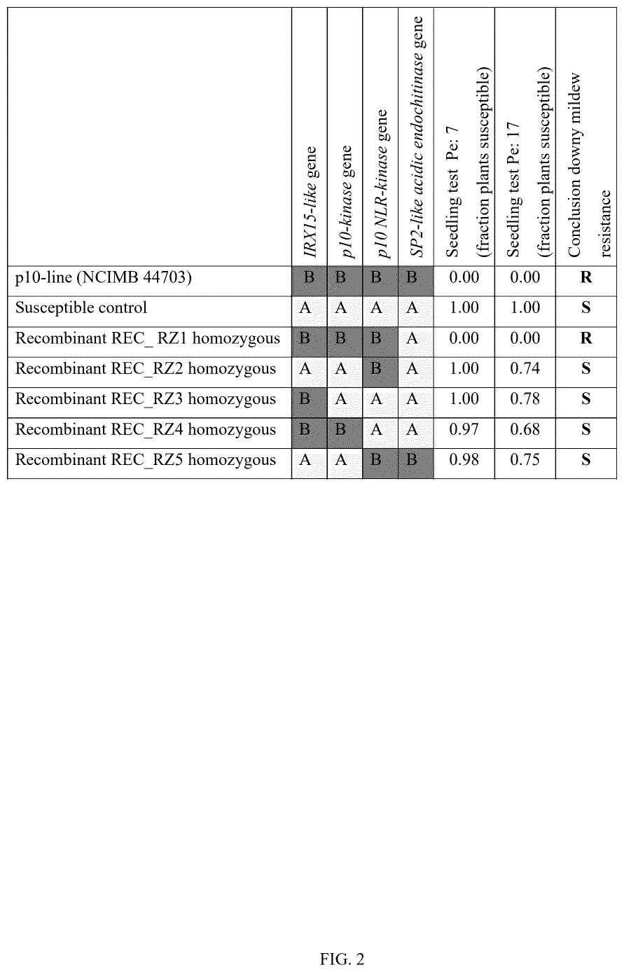

Through QTL mapping, the p10 locus of the invention was initially found to be located on chromosome 3, flanked by markers SO01770 (SEQ ID NO: 24 and SEQ ID NO: 25) and SO00979 (SEQ ID NO: 22 and SEQ ID NO: 23). New markers were developed within this QTL region and used for fine mapping using several populations derived from crosses between the resistant p10 line and different susceptible control lines. For fine-mapping, recombinants in the QTL region were identified and made homozygous and subsequently tested in bioassays for different Peronospora effusa races as described in example 1. Through many years of intensive fine-mapping, the QTL region for the p10 locus was reduced to a region of around 0.5 Mb on chromosome 3. Within this region 4 genes were identified that showed differences in sequence between the wildtype not downy mildew resistant controls and the resistant p10 line that were potentially functionally relevant: an IRX15-like gene (gene Spo22837 with genomic position chr3:13230167-13232246 on the public reference genome of Chinese spinach cultivar Sp75 (Xu et al. Nature Communications 8:15275, 2017)), a kinase gene (referred to as the p10-kinase gene from now on; gene Spo22843 with genomic position chr3:13356999-13357952 on the Sp75 reference genome), an NLR-kinase gene (referred to as the p10 NLR-kinase gene from now on; gene Spo22844 with genomic position chr3:13412057-13419839 on the Sp75 reference genome), and an SP2-like acidic endochitinase gene (gene Spo22849 with genomic position chr3:13465132-13466358 on the Sp75 reference genome). Further recombinants and double recombinants were identified within the region comprising these 4 genes, made homozygous and tested for resistance to many different Peronospora effusa races. The results of the bioassays for two Peronospora effusa races (Pe: 7 and Pe: 17), and the conclusions on downy mildew resistance from all bioassays performed are shown in FIG. 2.

FIG. 2 provides an analysis of the most relevant recombinants. Bioassays for resistance to Peronospora effusa were performed using seedling tests on control lines and several homozygous recombinants and double recombinants. For the controls (susceptible line and p10-line) 30 plants were tested, while for the recombinants between 30 and 125 plants were tested in each seedling test. This table shows the results of two representative bioassays for Peronospora effusa races Pe: 7 and Pe: 17 as a fraction of the plants in the test that are susceptible, and the conclusions on downy mildew resistance from all bioassays. In case no susceptible plants were seen in a seedling test, the fraction of the plants that were susceptible was 0.00. If for other downy mildew races the seedling tests showed similar results, the conclusion for that tested line was that it had a broad-spectrum resistance (R) to downy mildew. In case all or almost all plants in a seedling test were susceptible, and seedling tests for other downy mildew races showed similar results, the conclusion for that tested line was that it was susceptible (S) to downy mildew and did not have a broad-spectrum resistance to downy mildew.

The homozygous single recombinant REC_RZ1 demonstrates that plants without the SP2-like acidic endochitinase gene of the resistant p10-line are resistant to downy mildew. Based on this recombinant it can be concluded the SP-2 like acidic endochitinase gene is not required for the downy mildew resistance phenotype of the p10-locus.

The homozygous single recombinant REC_RZ3 demonstrates that plants that have the IRX15 gene of the resistant p10-line and lack the SP2-like acidic endochitinase gene, the p10 NLR-kinase gene and the p10-kinase gene of the resistant p10-line are susceptible to downy mildew. This shows the IRX15 gene is not responsible for the p10 downy mildew resistance phenotype.

Homozygous double recombinant REC_RZ4 has the p10-kinase gene of the resistant p10-line and lacks the p10 NLR-kinase gene of the resistant p10-line, while homozygous recombinants REC_RZ2 and REC_RZ5 have p10 NLR-kinase gene of the resistant p10-line and lack the p10-kinase gene of the resistant p10-line. All three recombinants are susceptible to downy mildew. These recombinants demonstrate that both the p10-kinase gene of the resistant p10-line and the p10 NLR-kinase gene of the resistant p10-line need to be homozygously present in a spinach plant for it to exhibit the broad-spectrum resistance of the present invention.

### Example 3—the Genes Responsible for the p10 Downy Mildew Resistance Phenotype

From the recombinants it can be concluded two genes are required for the p10 downy mildew resistance phenotype: the p10-kinase gene and the p10 NLR-kinase gene.

The coding sequence of the allele of the p10-kinase gene as found in the p10-line is provided as SEQ ID NO: 2, while the genomic sequence of the p10-kinase gene, including 2 kb upstream and 2 kb downstream, as present in the p10-line is provided as SEQ ID NO: 6. The coding sequence of the wildtype allele of the p10-kinase gene, not conferring downy mildew resistance, as e.g. found in Chinese spinach cultivar Sp75 (Xu et al. Nature Communications 8:15275, 2017) is provided as SEQ ID NO: 8, while the genomic sequence, including 2 kb upstream and 2 kb downstream, of the p10-kinase gene as found in cultivar Sp75 is provided as SEQ ID NO: 12. The p10-kinase gene as found in the p10-line differs from the p10-kinase gene as found in cultivar Sp75 by two deletions: (1) a 558 bp deletion (SEQ ID NO: 13) starting upstream of the start codon and deleting the first 288 bp of the coding sequence (SEQ ID NO: 14) and leading to a new start codon 318 bp after the start codon as found in the p10-kinase gene of Sp75 and (2) a 45 bp deletion within the coding sequence of the p10-kinase gene (SEQ ID NO: 15).

The wild type allele of the p10-kinase gene not conferring downy mildew resistance, as in the public spinach genome Sp75 (Xu et al. Nature Communications 8:15275, 2017), encodes a protein (SEQ ID NO: 10) of 317 amino acids long. This protein is predicted to be a cysteine-rich receptor-like protein kinase and is predicted to comprise a phosphorylase kinase domain at amino acid position 13-112 and a phosphotransferase domain at amino acid position 116-312.

The p10-kinase gene conferring downy mildew resistance as in a plant comprising the nucleic acid molecule of the invention, such as a plant grown from seed deposited with the NCIMB under deposit number NCIMB 42554, encodes a protein (SEQ ID NO: 4) of 196 amino acids long. This protein is predicted to comprise a phosphotransferase domain at amino acid position 10-191. It lacks the phosphorylase kinase domain that is predicted to be present in the wildtype, not downy mildew resistance conferring, allele as present in for example spinach cultivar Sp75.

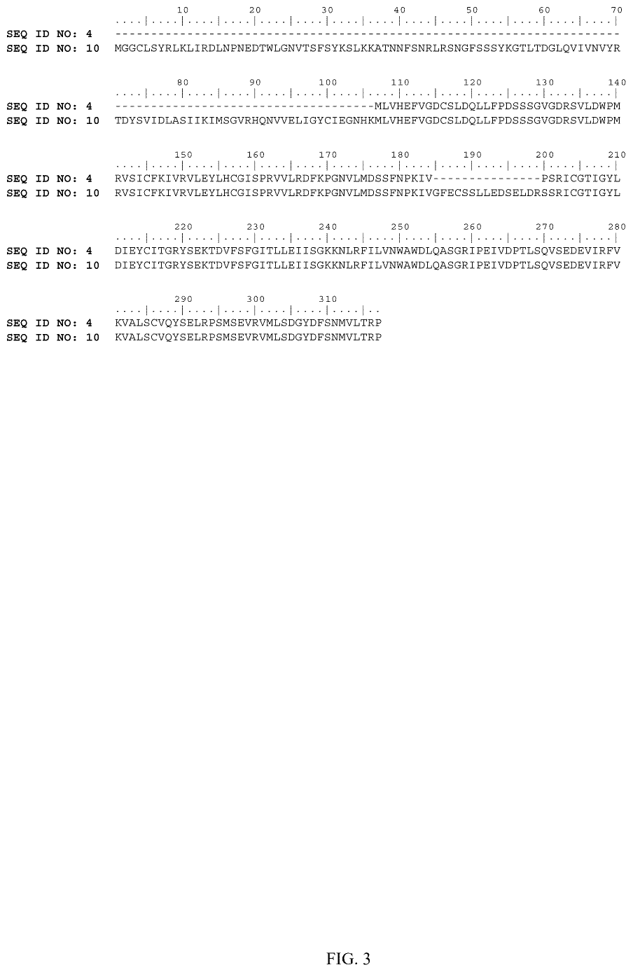

FIG. 3 shows the alignment of the protein sequences encoded by the downy mildew resistance conferring p10-kinase gene as found in plants of the p10-line (representative seeds having been deposited under deposit number NCIMB 42554) and the wildtype not downy mildew resistance conferring p10-kinase gene as present in plants of spinach cultivar Sp75. As is clear from this protein sequence alignment, the resistance conferring p10-kinase protein lacks the first 106 amino acids that are present in the wildtype p10-kinase protein as found in Sp75. This deletion results in the deletion of a phosphorylase kinase domain that is present in the wildtype p10-kinase protein as present in Sp75 and other material not comprising the p10 locus. Additionally, the resistance conferring p10-kinase protein has a 15 amino acid deletion and an S to P amino acid substitution at position 79 of SEQ ID NO: 4, when compared to the wildtype p10-kinase protein as present in Sp75.

The coding sequence of the allele of the p10 NLR-kinase gene as found in the p10-line is provided as SEQ ID NO: 1, while the genomic sequence of the p10 NLR-kinase gene, including 2 kb upstream and 2 kb downstream, as present in the p10-line is provided as SEQ ID NO: 5. The coding sequence of the wildtype allele of the p10 NLR-kinase gene, not conferring downy mildew resistance, as e.g. found in Chinese spinach cultivar Sp75 (Xu et al. Nature Communications 8:15275, 2017) is provided as SEQ ID NO: 7, while the genomic sequence, including 2 kb upstream and 2 kb downstream, of the p10 NLR-kinase gene as found in cultivar Sp75 is provided as SEQ ID NO: 11. The p10 NLR-kinase gene as found in the p10-line differs from the p10 NLR-kinase gene as found in cultivar Sp75 by a 6 bp insertion in the coding sequence.

The wild type allele of the p10 NLR-kinase gene not conferring downy mildew resistance, as in the public spinach genome Sp75, encodes a protein (SEQ ID NO: 3) of 1454 amino acids long. This protein is a Nucleotide-binding Leucine-rich Repeat protein with an integrated kinase domain (NLR-kinase). This NLR-kinase protein is predicted to comprise a phosphorylase kinase domain at amino acid position 16-130, a phosphotransferase domain at amino acid position 133-351, an NB-ARC domain at position 576-731 and a Leucine-rich repeat region at position 936-1411. The sequence of the integrated kinase domain (made up by the phosphorylase kinase domain and the phosphotransferase domain) is quite similar to the sequence of the wildtype pO-kinase as present in Sp75.

The p10 NLR-kinase gene conferring downy mildew resistance as in a plant comprising the nucleic acid molecule of the invention, such as a plant grown from seed deposited with the NCIMB under deposit number NCEVIB 42554, encodes a protein (SEQ ID NO: 9) of 1456 amino acids long. The only difference in sequence between the wildtype allele of the p10 NLR-kinase protein as present in Sp75 and the allele as present in the p10 line is a 2 amino acid insertion at amino acid position 412-413 of SEQ ID NO: 9, which is in between the integrated kinase domain and the NB-ARC domain. The 2 amino acid insertion is necessary for the p10 NLR-kinase protein to be able to contribute to the broad-spectrum downy mildew resistance of this invention.

### Example 4 Marker Development

Markers were developed on the sequence differences in the p10 NLR-kinase gene and p10-kinase gene between the p10-line and susceptible material such as reference variety Viroflay. Marker SORZ01 was designed on the 6 bp insertion present in the allele of the p10 NLR-kinase gene as found in the p10-line (see Table 1 and section Marker Information). Marker SORZ02 was developed on the INDEL in the p10-kinase gene: the allele of the p10-kinase gene as present in the p10-line (SEQ ID NO: 20, positions 58-63) has sequence TCCCAA, and the allele of the p10-kinase gene as present in the susceptible material like Viroflay and Sp75 (SEQ ID NO: 21, positions 56-108) has sequence TAGGTTTTGAGTGTTCAAGTCTTCTAGAAGACAGTGAATTAGACCGTAGTA (SEQ ID NO: 29) (see Table 1 and section Marker Information). These two markers can be used to identify spinach plants as plants comprising the nucleic acid molecule of the invention.

### Example 5 Introducing the Nucleic Acid Molecule of this Invention in a Plant

A spinach plant comprising the nucleic acid molecule of the invention, of which a representative sample of seed was deposited with the NCIMB under accession number NCIMB 42554 was crossed with a plant of variety Viroflay carrying no downy mildew resistance alleles to obtain a F1 generation. Subsequently, an F1 plant was selfed to obtain an F2 population.

Plants of the F2 population were assayed as described in Example 1 for resistance to Peronospora effusa Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20. Approximately 25% of the plants scored intermediately resistant in the assay, all other plants scored susceptible. This segregation pattern is consistent with that of a recessive inheritance.

Genomic DNA of each plant of the same F2 population was isolated and used for marker analysis with markers SORZ01 and SORZ02. Approximately 25% of the plants were homozygous for marker alleles SEQ ID NO: 18 and SEQ ID NO: 20. The plants that were homozygous for marker alleles SEQ ID NO: 18 and SEQ ID NO: 20 completely correlated with the plants that scored intermediately resistant to Pe: 1, Pe: 2, Pe: 3, Pe: 4, Pe: 5, Pe: 6, Pe: 7, Pe: 8, Pe:9, Pe: 10, Pe: 11, Pe: 12, Pe: 13, Pe: 14, Pe: 15, Pe: 16, Pe: 17, Pe: 18, Pe:19 and Pe: 20. The plants that were heterozygous for marker alleles SEQ ID NO: 18 and SEQ ID NO: 20 or homozygous for marker alleles SEQ ID NO: 19 and SEQ ID NO: 21 correlated with the plants that scored susceptible for Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

Alternatively, the genomic DNA of the plants of the F2 population can be used to sequence the p10 NLR-kinase gene to determine whether a genomic sequence is present having at least 95% sequence identity to SEQ ID NO: 11 (gDNA sequence of the wild type p10 NLR-kinase gene) and comprising an insertion of sequence motif GAAATA between nucleotides 5940 and 5941 of SEQ ID NO: 11. The genomic DNA of the plants of the F2 population can also be used to sequence the p10-kinase gene, to determine whether a genomic sequence having at least 95% sequence identity to SEQ ID NO: 6 is present. In other words, the genomic sequence of the p10-kinase gene can be checked for presence of the two deletions of SEQ ID NO: 13 and SEQ ID NO: 15. Plants that are homozygous for a genomic sequence of the p10 NLR-kinase gene comprising the insertion of the sequence motif and homozygous for a genomic sequence of the p10-kinase gene comprising the deletions of SEQ ID NO: 13 and optionally SEQ ID NO: 15 are plants that are at least intermediately resistant to Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

The invention is further described by the following numbered paragraphs:

1. An agronomically elite Spinacia oleracea plant comprising a nucleic acid molecule which when homozygously present provides at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, wherein said nucleic acid molecule comprises:
a) a gene which comprises a coding sequence according to SEQ ID NO: 1 and a gene which comprises a coding sequence according to SEQ ID NO: 2, or
b) a gene which comprises a coding sequence having at least 95.0% sequence identity to SEQ ID NO: 7 and comprises an insertion of sequence motif GAAATA between nucleotides 1235 and 1236 of SEQ ID NO: 7 and a gene which comprises a coding sequence having at least 95.0% sequence identity to SEQ ID NO: 8 and lacks a sequence according to SEQ ID NO: 14, wherein the 95.0% sequence identity is over the part of SEQ ID NO: 8 without the lacking sequence, or
c) a gene which comprises a coding sequence having at least 95.0% sequence identity to SEQ ID NO: 1 and comprises an insertion of sequence motif GAAATA between nucleotides 1235 and 1236 of SEQ ID NO: 7 and a gene which comprises a coding sequence having at least 95.0% sequence identity to SEQ ID NO: 2, or
d) a gene which comprises a genomic sequence having at least 95.0% sequence identity to SEQ ID NO: 11 and comprises an insertion of sequence motif GAAATA between nucleotides 5940 and 5941 of SEQ ID NO: 11 and a gene which comprises a genomic sequence having at least 95.0% sequence identity to SEQ ID NO: 6, or
e) a gene encoding a protein having an amino acid sequence according to SEQ ID NO: 3, and a gene encoding a protein having an amino acid sequence according to SEQ ID NO: 4, or
f) a gene encoding a protein having an amino acid sequence that has at least 95.0% sequence identity to SEQ ID NO: 9 and comprises an insertion of amino acids IE between amino acids 411 and 412 of SEQ ID NO: 9 and a gene encoding a protein having an amino acid sequence that has at least 95.0% sequence identity to SEQ ID NO: 4.

2. The plant of paragraph 1, wherein the nucleic acid molecule is as present in the genome of a Spinacia oleracea plant, representative seed of which was deposited with the NCIMB under accession number NCIMB 42554.

3. The plant of paragraph 1 or paragraph 2, wherein the nucleic acid molecule is introgressed from a plant, which is grown from seed deposited with the NCIMB under accession number 42554 or a progeny plant of seed of NCIMB accession 42554 that has retained the nucleic acid molecule.

4. The plant of any one of paragraphs 1 to 3, wherein the nucleic acid molecule is homozygously present.

5. A Spinacia oleracea seed comprising the nucleic acid molecule as defined in paragraph 1 in its genome, wherein a plant grown from the seed is the plant of any one of paragraphs 1 to 4.

6. Propagation material capable of developing into and/or being derived from the Spinacia oleracea plant according to any one of paragraphs 1 to 4, wherein the propagation material is suitable for sexual reproduction, and is in particular selected from the group comprising a microspore, pollen, an ovary, an ovule, an embryo sac, and an egg cell; or is suitable for vegetative reproduction, and is in particular selected from the group comprising a cutting, a root, a stem, a cell, and a protoplast; or is suitable for tissue culture of regenerable cells, and is in particular selected from the group comprising a leaf, pollen, an embryo, a cotyledon, a hypocotyl, a meristematic cell, a root, a root tip, an anther, a flower, a seed, and a stem; wherein the plant developed from the propagation material comprises the nucleic acid molecule as defined in paragraph 1.

7. A method for selecting a Spinacia oleracea plant that is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising identifying the presence of the nucleic acid molecule as defined in paragraph 1 and selecting the Spinacia oleracea plant homozygously comprising said nucleic acid molecule.

8. The method of paragraph 7, wherein the nucleic acid molecule comprises SEQ ID NO: 1 and SEQ ID NO: 2.

9. The method of paragraph 7 or paragraph 8, wherein the presence of the nucleic acid molecule is identified by using markers SEQ ID NO: 18 and/or SEQ ID NO: 20.

10. A method for determining the genotype of a Spinacia oleracea plant comprising the nucleic acid molecule of seed deposited with the NCIMB under accession number 42554, comprising detecting in the Spinacia oleracea plant the presence of the nucleic acid molecule as defined in paragraph 1 and selecting the Spinacia oleracea plant homozygously comprising said nucleic acid molecule.

11. The method of paragraph 10, wherein the nucleic acid molecule comprises SEQ ID NO: 1 and SEQ ID NO: 2.

12. The method of paragraph 10 or paragraph 11, wherein the presence of the nucleic acid molecule is identified by using markers SEQ ID NO: 18 and/or SEQ ID NO: 20.

13. A method for producing an agronomically elite Spinacia oleracea plant comprising at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe:

18, Pe: 19 and Pe: 20, comprising:
(a) crossing a first plant selected as comprising the nucleic acid molecule by the method of any one of paragraphs 7 to 9, with a second plant; and
(b) performing one or more rounds of selfing and/or crossing.

14. The method of paragraph 13, further comprising: (c) selecting after each round of selfing or crossing for a plant that comprises said resistance.

15. A method for producing an agronomically elite Spinacia oleracea plant exhibiting at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising:
a) crossing a first parent plant as defined in any one of paragraphs 1 to 4 with a second parent plant to obtain an F1 population;
b) performing one or more rounds of selfing and/or crossing of the plant resulting from the cross to obtain a further generation population;
c) selecting from among the plants resulting from the further generation population of step b) a plant that homozygously comprises the nucleic acid molecule as defined in paragraph 1, which plant is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

16. A method for the production of hybrid seed of a Spinacia oleracea plant comprising crossing a first parent plant with a second parent plant, wherein the first and the second parent plant comprise the nucleic acid molecule as defined in paragraph 1, and wherein the homozygous presence of said nucleic acid molecule leads to at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20 in a plant that is grown from the hybrid seed.

17. A method for producing a Spinacia oleracea plant exhibiting at least intermediate resistance to Peronospora effusa, comprising the step of introducing the nucleic acid molecule as defined in paragraph 1, wherein the nucleic acid molecule when homozygously present in a Spinacia oleracea plant confers at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe:20.

18. The method according to paragraph 17, wherein the nucleic acid molecule is introduced by cisgenesis.

19. A method for producing an agronomically elite Spinacia oleracea plant comprising at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising:
(a) crossing a first plant determined to have the genotype of a Spinacia oleracea plant comprising the nucleic acid molecule by the method of any one of paragraphs 10 to 12, with a second plant; and
(b) performing one or more rounds of selfing and/or crossing.

20. The method of paragraph 19, further comprising: (c) selecting after each round of selfing or crossing for a plant that comprises said resistance.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An agronomically elite Spinacia oleracea plant comprising a nucleic acid molecule which when homozygously present provides at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, wherein said nucleic acid molecule comprises:
a) a gene which comprises a coding sequence according to SEQ ID NO: 1 and a gene which comprises a coding sequence according to SEQ ID NO: 2, or
b) a gene which comprises a coding sequence having at least 95.0% sequence identity to SEQ ID NO: 7 and comprises an insertion of sequence motif GAAATA between nucleotides 1235 and 1236 of SEQ ID NO: 7 and a gene which comprises a coding sequence having at least 95.0% sequence identity to SEQ ID NO: 8 and lacks a sequence according to SEQ ID NO: 14, wherein the 95.0% sequence identity is over the part of SEQ ID NO: 8 without the lacking sequence, or
c) a gene which comprises a coding sequence having at least 95.0% sequence identity to SEQ ID NO: 1 and comprises an insertion of sequence motif GAAATA between nucleotides 1235 and 1236 of SEQ ID NO: 7 and a gene which comprises a coding sequence having at least 95.0% sequence identity to SEQ ID NO: 2, or
d) a gene which comprises a genomic sequence having at least 95.0% sequence identity to SEQ ID NO: 11 and comprises an insertion of sequence motif GAAATA between nucleotides 5940 and 5941 of SEQ ID NO: 11 and a gene which comprises a genomic sequence having at least 95.0% sequence identity to SEQ ID NO: 6, or
e) a gene encoding a protein having an amino acid sequence according to SEQ ID NO: 3, and a gene encoding a protein having an amino acid sequence according to SEQ ID NO: 4, or
f) a gene encoding a protein having an amino acid sequence that has at least 95.0% sequence identity to SEQ ID NO: 9 and comprises an insertion of amino acids IE between amino acids 411 and 412 of SEQ ID NO: 9 and a gene encoding a protein having an amino acid sequence that has at least 95.0% sequence identity to SEQ ID NO: 4.

2. The plant of claim 1, wherein the nucleic acid molecule is as present in the genome of a Spinacia oleracea plant, representative seed of which was deposited with the NCIMB under accession number NCIMB 42554.

3. The plant of claim 1, wherein the nucleic acid molecule is introgressed from a plant, which is grown from seed deposited with the NCIMB under accession number 42554 or a progeny plant of seed of NCIMB accession 42554 that has retained the nucleic acid molecule.

4. The plant of claim 1, wherein the nucleic acid molecule is homozygously present.

5. A Spinacia oleracea seed comprising the nucleic acid molecule as defined in claim 1 in its genome, wherein a plant grown from the seed is the plant of claim 1.

6. Propagation material capable of developing into and/or being derived from the Spinacia oleracea plant according to claim 1, wherein the propagation material is suitable for sexual reproduction, and is in particular selected from the group comprising a microspore, pollen, an ovary, an ovule, an embryo sac, and an egg cell; or is suitable for vegetative reproduction, and is in particular selected from the group comprising a cutting, a root, a stem, a cell, and a protoplast; or is suitable for tissue culture of regenerable cells, and is in particular selected from the group comprising a leaf, pollen, an embryo, a cotyledon, a hypocotyl, a meristematic cell, a root, a root tip, an anther, a flower, a seed, and a stem; wherein the plant developed from the propagation material comprises the nucleic acid molecule as defined in claim 1.

7. A method for selecting a Spinacia oleracea plant that is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising identifying the presence of the nucleic acid molecule as defined in claim 1 and selecting the Spinacia oleracea plant homozygously comprising said nucleic acid molecule.

8. The method of claim 7, wherein the nucleic acid molecule comprises SEQ ID NO: 1 and SEQ ID NO: 2.

9. The method of claim 7, wherein the presence of the nucleic acid molecule is identified by using markers SEQ ID NO: 18 and/or SEQ ID NO: 20.

10. A method for determining the genotype of a Spinacia oleracea plant comprising the nucleic acid molecule of seed deposited with the NCIMB under accession number 42554, comprising detecting in the Spinacia oleracea plant the presence of the nucleic acid molecule as defined in claim 1 and selecting the Spinacia oleracea plant homozygously comprising said nucleic acid molecule.

11. The method of claim 10, wherein the nucleic acid molecule comprises SEQ ID NO: 1 and SEQ ID NO: 2.

12. The method of claim 10, wherein the presence of the nucleic acid molecule is identified by using markers SEQ ID NO: 18 and/or SEQ ID NO: 20.

13. A method for producing an agronomically elite Spinacia oleracea plant comprising at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising:
(a) crossing a first plant selected as comprising the nucleic acid molecule by the method of claim 7, with a second plant; and
(b) performing one or more rounds of selfing and/or crossing.

14. The method of claim 13, further comprising: (c) selecting after each round of selfing or crossing for a plant that comprises said resistance.

15. A method for producing an agronomically elite Spinacia oleracea plant exhibiting at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising:
a) crossing a first parent plant, wherein the first parent plant is the plant of claim 1, with a second parent plant to obtain an F1 population;
b) performing one or more rounds of selfing and/or crossing of the plant resulting from the cross to obtain a further generation population;
c) selecting from among the plants resulting from the further generation population of step b) a plant that homozygously comprises the nucleic acid molecule as defined in claim 1, which plant is at least intermediate resistant to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

16. A method for the production of hybrid seed of a Spinacia oleracea plant comprising crossing a first parent plant with a second parent plant, wherein the first and the second parent plant comprise the nucleic acid molecule as defined in claim 1, and wherein the homozygous presence of said nucleic acid molecule leads to at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20 in a plant that is grown from the hybrid seed.

17. A method for producing a Spinacia oleracea plant exhibiting at least intermediate resistance to Peronospora effusa, comprising the step of introducing the nucleic acid molecule as defined in claim 1, wherein the nucleic acid molecule when homozygously present in a Spinacia oleracea plant confers at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20.

18. The method according to claim 17, wherein the nucleic acid molecule is introduced by cisgenesis.

19. A method for producing an agronomically elite Spinacia oleracea plant comprising at least intermediate resistance to Peronospora effusa races Pe: 1, Pe: 2, Pe: 3, Pe: 4, Pe: 5, Pe: 6, Pe: 7, Pe: 8, Pe: 9, Pe: 10, Pe: 11, Pe: 12, Pe: 13, Pe: 14, Pe: 15, Pe: 16, Pe: 17, Pe: 18, Pe: 19 and Pe: 20, comprising:
(a) crossing a first plant determined to have the genotype of a Spinacia oleracea plant comprising the nucleic acid molecule by the method of claim 10, with a second plant; and
(b) performing one or more rounds of selfing and/or crossing.

20. The method of claim 19, further comprising: (c) selecting after each round of selfing or crossing for a plant that comprises said resistance.
